(19) United States
(12) Patent Application Publication
(10) Pub. No.: US 2025/0179240 A1
(43) Pub. Date: Jun. 5, 2025

(54) A polyester polymer compound, preparation method and use thereof

(71) Applicant: ShanghaiTech University, Shanghai (CN)

(72) Inventors: Bolin LIN, Shanghai (CN); Jialin XU, Shanghai (CN); Luyan XU, Shanghai (CN)

(73) Assignee: ShanghaiTech University, Shanghai (CN)

(21) Appl. No.: 18/713,197

(22) PCT Filed: Nov. 24, 2022
(86) PCT No.: PCT/CN2022/134072

§ 371 Date: Feb. 21, 2025

(30) Foreign Application Priority Data
Nov. 25, 2021 (CN) 202111416142.2
Dec. 17, 2021 (CN) 202111553641.6
Mar. 17, 2022 (CN) 202210268028.8

(51) Int. Cl. C08G 63/08; C07D 309/30; C08G 63/82

(52) U.S. Cl. CPC C08G 63/08; C07D 309/30; C08G 63/823

(57) ABSTRACT

The present disclosure relates to the technical field of polymer synthesis, and in particular, to a polyester polymer compound and a preparation method thereof, the method employs a heterocyclic lactone of formula 1 as a raw material and carries out a ring-opening polymerization reaction catalyzed by an organic base to obtain the polyester polymer compound of formula 2 and/or formula 3. The present disclosure also discloses δLH₂ and a preparation method thereof. The method described in the present disclosure realizes the preparation of these polyester polymer compounds for the first time. The method in the present disclosure utilizes a wide range of raw materials and a simple, cost-effective catalyst with excellent activity. The present disclosure effectively alleviates the harm caused by the greenhouse effect. The present disclosure also discloses the use of these polyester polymer compounds, where the prepared polymer film is colorless, transparent, and has good flexibility, viscoelasticity, and the like. The polyester polymer compounds have good physicochemical properties and recyclability, and are promising for a wide range of applications.

### TECHNICAL FIELD

The present disclosure belongs to the technical field of polymer synthesis, relates to a polyester polymer compound, a preparation method and a use thereof, and in particular, relates to a CO₂-based chemical recyclable polymer based on CO₂, H₂, and 1,3-butadiene, a preparation method and a use thereof.

### BACKGROUND OF THE INVENTION

Synthetic polymers, as a common material, have become ubiquitous in various aspects of human life, including clothing, food packaging, housing, and transportation. It is predicted that the global production of polymers will cumulatively reach 34 billion tons from 1950 to 2050, and the vast majority of these polymers are difficult to be degraded due to their stable carbon-carbon bonded skeletons. The extensive utilization of non-biodegradable polymers follows a pattern of “raw material-monomer-polymer-material-waste,” contributing significantly to the severe plastic waste pollution and the loss of valuable materials.

Chemical recycling is a promising strategy to reduce the environmental and economic impacts of polymer materials. Chemical recycling can form a materials economy loop including synthesis, utilization, and recycling. This recycling economy mode is meaningful for reducing white pollution. Meanwhile, the synthesis of chemical recyclable polymers from cheap and readily available bulk chemicals, such as CO₂ and olefins, can decrease the cost and facilitate industrial mass production. Therefore, developing efficient conversion and utilization of CO₂ is of great significance for industrial production and sustainable development.

As a cheap, readily available, and renewable C1 source, CO₂ is well suited for the synthesis of a wide range of polymer materials, including polycarbonates, polyurethanes, polyureas, and polyesters. The synthesis of new polymers from CO₂ and cheap bulk chemicals (especially ethylene and 1,3-butadiene) has been developed since the 1970s. According to research of Inoue, Behr, and Beller, a six-membered cyclic lactone intermediate 3-ethylidene-6-vinyltetrahydro-2H-pyran-2-one (δ-L) containing two carbon-carbon double bonds is generated via a Pd-catalyzed coupling reaction between CO₂ and 1,3-butadiene. Then, in 2014, Nozaki reported the free radical polymerization of δ-L and three different topological polyolefins with side chains containing ester bonding functional groups. Shortly thereafter, Lin Bolin reported that monomers could be directly free-radically polymerized under air-only initiation, solvent-free, and additive-free conditions, and a fourth topological structure (8-structure) was found in the study. Later, a diethyl-substituted six-membered cyclic lactone, i.e., 3,6-diethyl-tetrahydro-2h-pyran-2-one (HL), synthesized from CO₂ and 1,3-butadiene was reported, however, the disubstituted caprolactone represented by HL was traditionally considered as a non-polymerizable monomer.

To enable large-scale production of CO₂-based polymers, several conditions must be met: 1. raw materials for copolymerization with CO₂ should be inexpensive bulk chemical materials to meet economic requirements for large-scale production; 2. the polymers produced should be easily processed into desired forms and possess commercially viable performance characteristics; 3. to address the growing issue of plastic pollution, the new polymers must be degradable. The commercially available CO₂-based polymers, mainly polycarbonates and polyols, fail to meet the requirements of cost, material performance, and degradability simultaneously. Additionally, the amount of CO₂ utilized in the production of these polymers is insufficient to effectively reduce CO₂ emissions. To establish the advantages of CO₂-based polymers in terms of cost, material performance, and closed-loop circular economy, and to achieve significant emission reductions, there is an urgent need to design and synthesize a novel CO₂-based polymer with a unique structure, low cost, and potential for industrialization.

### SUMMARY OF THE INVENTION

The present disclosure, for the first time, provides a polyester polymer compound and a preparation method thereof, where the polyester polymer compound is obtained through the ring-opening polymerization (ROP) of heterocyclic lactones, for example, polyHL is obtained through the ring-opening polymerization (ROP) of diethyl-substituted six-membered cyclic lactone (i.e., 3,6-diethyl-tetrahydro-2h-pyran-2-one (HL)) derived from CO₂, H₂, and 1,3-butadiene. The present disclosure features simple preparation steps, high yield, and low cost. The prepared polyester has high molecular weight and exceptional performance, such as high transparency and high stability. Moreover, a cyclic polymer synthesized though anionic ring-opening polymerization of heterocyclic lactones in the present disclosure can be degraded back to the lactone monomer HL under specific conditions (FIGS. 15˜16). The present disclosure also provides a use of the polyester polymer compound.

The present disclosure provides a polyester polymer compound, comprising repeating units shown as formula 4 below:

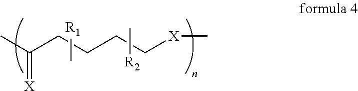

where R₁, R₂ are each independently selected from any one of hydrogen, halogen, alkyl, substituted alkyl (linear alkyl, branched alkyl, cycloalkyl), alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; where R₁ and R₂ are the same or different and exist simultaneously;
where the alkyl, alkenyl, alkynyl, monocyclic aromatic group, polycyclic aromatic group, and polyheterocyclic aromatic group are mono-substituted or poly-substituted; and where the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide group, sulfonamide group, morpholinyl, and piperazinyl;
where X is a heteroatom; and where n represents the number of repeating units in formula 4, n is a positive integer no less than 1.

In a preferred embodiment, the polyester polymer compound is a carbon dioxide-based polyester poly(δLH₂), and the carbon dioxide-based polyester poly(δLH₂) comprises repeating units shown as formula 6 below:

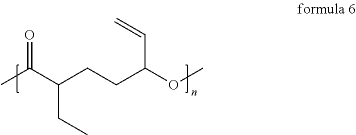

where n is a positive integer greater than or equal to 1.

The polyester polymer compound in the present disclosure comprises a linear, cyclic, star-shaped topology.

In a preferred embodiment, the polyester polymer compound has a structure shown as formula 2 when it is linear:

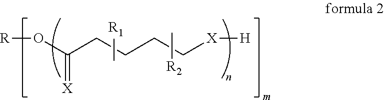

R₁, R₂, X, n, and m are defined as described in the compound of formula 4 above,
where R is selected from any one of alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol.

In a further preferred embodiment, when the polyester polymer compound is a carbon dioxide-based polyester poly(δLH₂), the carbon dioxide-based polyester poly(δLH₂) has a structure shown as formula 7:

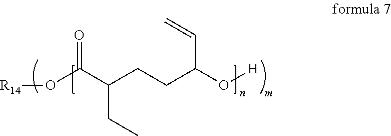

where R₁₄ is selected from C1-C20 alkyl, substituted C₁-C₂₀ alkyl, C2-C20 alkenyl, substituted C₂-C₂₀ alkenyl, C2-C20 alkynyl, substituted C₂-C₂₀ alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
where n is a positive integer no less than 1,
and m is a positive integer no less than 1.

In a preferred embodiment, the polyester polymer compound has a structure shown as formula 3 when it is cyclic:

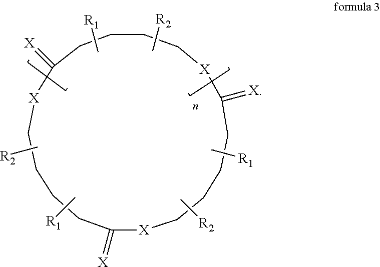

Another aspect of the present disclosure provides a use of the polyester polymer compound in the preparation of one or more of polyurethanes, polymer films, pressure sensitive adhesives, adhesive tapes, and thermoplastic elastomers.

In a preferred embodiment, the present disclosure provides a use of the polyester polymer compound having a linear structure in the preparation of polyurethanes or pressure sensitive adhesives.

In a preferred embodiment, the present disclosure provides a use of the polyester polymer compound having a cyclic structure in the preparation of polymer films, pressure sensitive adhesives, or thermoplastic elastomers. In a specific embodiment, a polyester polymer compound product is a polymer film. In a specific embodiment, a polyester polymer compound product is an adhesive tape.

Another aspect of the present disclosure provides a method for preparing a polyester polymer compound, comprising one or more of the follows:
A) when the polyester polymer compound is linear, the method comprises: obtaining the polyester polymer compound of formula 2 by synthesis using heterocyclic lactones of formula 1 as raw materials under the catalysis of an organic base and the initiation of an initiating reagent R(OH)ₘ capable of providing active protons; where a corresponding reaction is shown as reaction formula I:

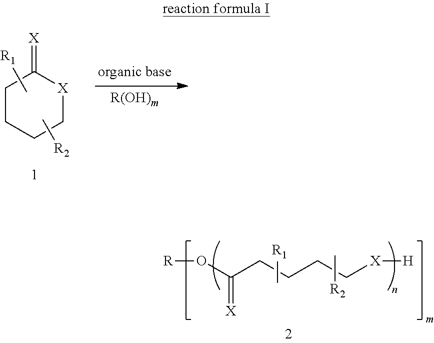

B) when the polyester polymer compound is cyclic, the method comprises: obtaining the polyester polymer compound of formula 3 by synthesis using heterocyclic lactones of formula 1 as raw materials under the catalysis of an organic base; where a corresponding reaction is shown as reaction formula II:

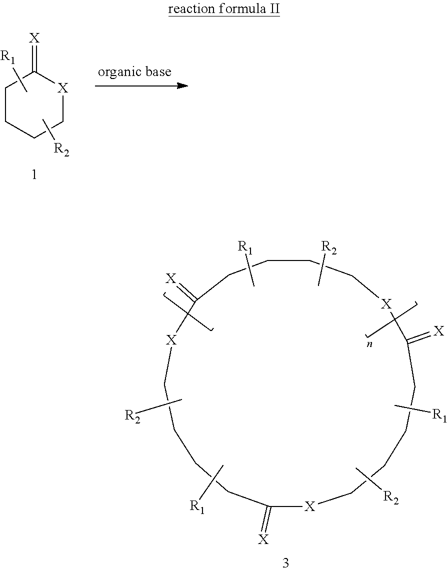

where in A) and/or B), R₁, R₂ are each independently selected from any one of hydrogen, halogen, alkyl, substituted alkyl (linear alkyl, branched alkyl, cycloalkyl), alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; where R₁ and R₂ are the same or different and exist simultaneously;
where the alkyl, alkenyl, alkynyl, monocyclic aromatic group, polycyclic aromatic group, and polyheterocyclic aromatic group are mono-substituted or poly-substituted; and where the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide group, sulfonamide group, morpholinyl, and piperazinyl;
where X is a heteroatom;
where n is a positive integer no less than 1,
where the organic bases comprise phosphazene, a compound containing a guanidine group, and a compound containing an amidino group;
where in B), R is selected from alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
and m is a positive integer no less than 1.

In a preferred embodiment, in A), when the polyester polymer compound is a carbon dioxide-based polyester poly(δLH₂), the method comprises: obtaining the carbon dioxide-based polyester poly(δLH₂) of formula 7 by synthesis using δLH₂ of formula 8 as raw materials under the catalysis of an organic base and the initiation of initiation reagents R₁₄ (OH)ₘ and/or (MO)ₘR₁₄ capable of providing active protons; where a corresponding reaction is shown as reaction formula III:

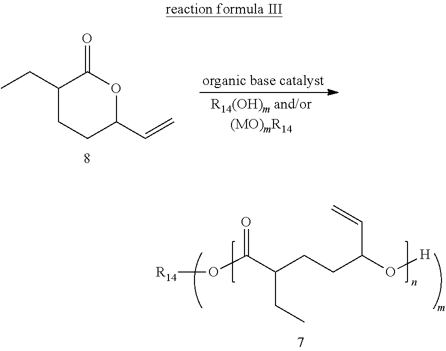

where R₁₄ is selected from C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
M is K, Na, Li, Rb or Cs;
where n is a positive integer no less than 1,
and m is a positive integer no less than 1;
and wherein the organic base is selected from one or more of phosphazene, a compound containing a guanidine group, and a compound containing an amidino group.

In a preferred embodiment, the phosphazene is a strong Lewis base having a structure of (R₂N)₂₋P═N; preferably, the phosphazene has a structure shown as formula 5:

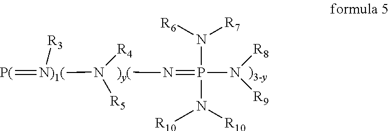

where in formula 5, R₃—R₁₀ each is independently selected from C1-C10 alkyl; and y is a positive integer no less than 1.

In a preferred embodiment, the phosphazene is selected from ^{t}Bu-P₁, ^{t}Bu-P₂, and ^{t}Bu-P₄, their corresponding structures are shown below, respectively:

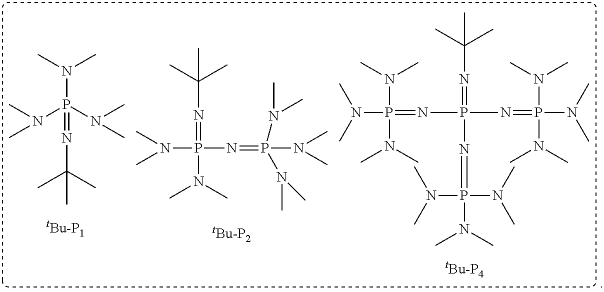

Another aspect of the present disclosure provides a polyester polymer compound, where the polyester polymer compound is synthesized by the methods described above.

Another aspect of the present disclosure provides a use of an organic base in the catalysis of heterocyclic lactones of formula 1 to prepare the polyester polymer compound of formula 2 and/or the polyester polymer compound of formula 3, where the organic base comprises phosphazene, a compound containing a guanidine group, and/or a compound containing an amidino group; where the structure of the heterocyclic lactones is shown as formula 1, and the structures of the polyester polymer compound are shown as formula 2 and/or formula 3, respectively:

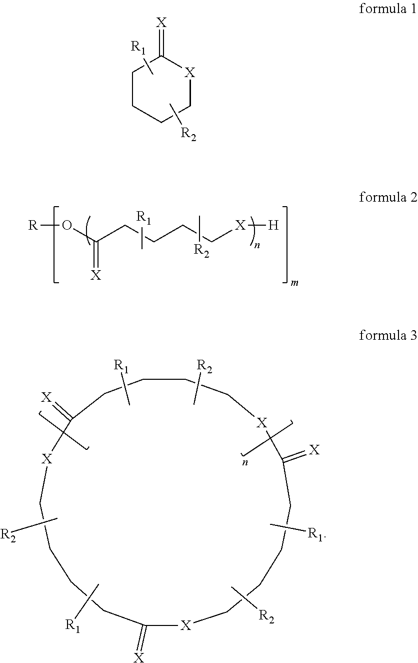

Preferably, the present disclosure provides a use of the organic base in the catalysis of δLH₂ of formula 8 to prepare carbon dioxide-based polyester poly(δLH₂) of formula 7, where the structure of δLH₂ is shown as formula 8, and the structure of the carbon dioxide-based polyester poly(δLH₂) is shown as formula 7, respectively:

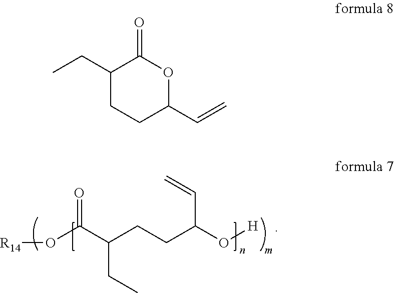

Preferably, the organic base is phosphazene, and the phosphazene has a structure shown as formula 5 below:

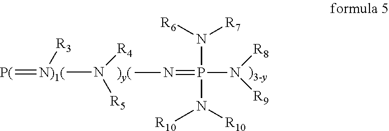

wherein in formula 5, R₃—R₁₀ each is independently selected from C1-C10 alkyl;
and y is 1, 2, or 3.

Another aspect of the present disclosure provides a method for catalyzing the recovery of heterocyclic monomers from a polyester polymer compound of formula 2 and/or formula 3, comprising one or more of the follows:
a) catalyzing the polyester polymer compound of formula 2 using a catalytic method or a pyrolysis method;
b) catalyzing the polyester polymer compound of formula 3 using an inorganic salt pyrolysis catalytic method or a La[N(SiMe₃)₂]₃ mild catalytic method;
in a), the catalytic method comprises: degrading the polyester polymer compound of formula 2 by using an inorganic salt, a metal organic compound, or an organic compound catalyst, to obtain heterocyclic monomers of formula 1, or an oligomer or a derivative thereof;
the pyrolysis method comprises: pyrolyzing the polyester polymer compound of formula 2 to obtain the heterocyclic monomers of formula 1, or an oligomer or a derivative thereof;
in b), the inorganic salt pyrolysis catalytic method comprises: degrading the polyester polymer compound of formula 3 under the catalysis of an inorganic salt to obtain the heterocyclic monomers of formula 1, or an oligomer or a derivative thereof;
the La[N(SiMe₃)₂]₃ mild catalytic method comprises: degrading the polyester polymer compound of formula 3 under the catalysis of La[N(SiMe₃)₂]₃ under mild conditions, to obtain the heterocyclic monomers of formula 1, or an oligomer or a derivative thereof.

Preferably, in a), when the polyester polymer compound is carbon dioxide-based polyester poly(δLH₂);

the catalytic method comprises: degrading the carbon dioxide-based polyester poly(δLH₂) of formula 8 by using an inorganic salt, a metal organic compound, or an organic compound catalyst, to obtain δLH₂ of formula 7, or an oligomer or a derivative thereof;

the pyrolysis method comprises: pyrolyzing the carbon dioxide-based polyester poly(δLH₂) of formula 8 to obtain δLH₂ of formula 7, or an oligomer or a derivative thereof.

Another aspect of the present disclosure provides a method for preparing a disubstituted α,β-saturated six-membered cyclic lactone, when the polyester polymer compound is carbon dioxide-based polyester poly(δLH₂), the method comprises using disubstituted α,β-unsaturated six-membered cyclic lactones of formula 9 as raw materials, Stryker reagent or a mixture of reagents capable of generating Stryker reagent in situ as a catalyst, and organosilane as a hydrogen source to perform a selective reduction reaction of conjugated olefins, so as to obtain the disubstituted α,β-saturated six-membered cyclic lactone of formula 10, where the selective reduction reaction is shown as reaction formula A:

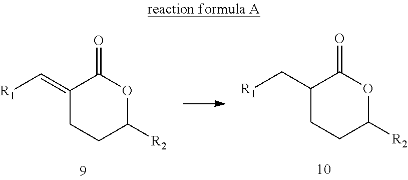

where R₁, R₂ are each independently selected from any one of hydrogen, halogen, C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; wherein R¹ and R₂ are the same or different.

Another aspect of the present disclosure provides a use of the method for preparing δLH₂ as described above in the preparation of the carbon dioxide-based polyester poly(δLH₂) of formula 8, polyurethanes, or pressure-sensitive adhesives. The disubstituted α,β-saturated six-membered cyclic lactone has a structure shown in formula 10:

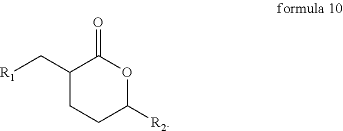

As described above, the CO₂-based polyester polymer compound of the present disclosure, the preparation method and use thereof have the following beneficial effects.

The method for the preparation of CO₂-based polyester polymer compounds in the present disclosure employs the heterocyclic monomer of Formula 1 as a raw material, and provides two initiation modes: 1. an organic base as a catalyst, and a reagent capable of providing reactive protons, such as R(OH)ₘ, as an initiator; 2. an alkoxide alone as an initiator, in which a ring-opening polymerization reaction is performed at, for example, −100˜200° C., and controllable polyester polymer compounds can then be obtained by this one-step method. This method is simple and the raw materials are easy to obtain. The raw materials include cheap carbon dioxide gas, which can effectively alleviate the greenhouse effect. The molecular weight of the polyester polymer compounds can be changed by adjusting the ratio of the raw materials, the reaction temperature, the reaction time, and other conditions. The method of the present disclosure produces polymers with excellent physical and chemical properties (such as mechanical performance) and recyclability through ring-opening polymerization, thereby increasing their potential applications.

The raw material used in the method of the present disclosure, heterocyclic lactone, is widely available and simple to prepare. When the heterocyclic lactone is HL, it can be prepared from CO₂ and 1,3-butadiene, which in turn alleviates the hazards of the greenhouse effect caused by CO₂. The catalyst used in the method of the present disclosure has good catalytic activity, and the preparation method thereof is simple and inexpensive.

The method of the present disclosure is capable of synthesizing cyclic polymers with ultra-high molecular weight and medium molecular weight distribution, and in some embodiments, the cyclic polymers have a molecular weight of up to 613.8 kg mol⁻¹ and a molecular weight distribution Ð of 1.45. The cyclic polymers can be used to prepare a variety of polymer products, such as polymer films, adhesive tapes, etc.; the prepared polymer films are colorless, transparent, and have good properties such as flexibility and viscoelasticity.

The method provided by the present disclosure can effectively obtain monomers through chemical recycling of various cyclic polymers including, but not limited to, cyclic polymers prepared in the present disclosure. Therefore, the method has the prospect of wide application and suits various monomers.

The method for the preparation of CO₂-based polyester poly(δLH₂) in the present disclosure employs the δLH₂ of formula 1 as a raw material, and provides two initiation modes: 1. an organic base as a catalyst, and a reagent capable of providing reactive protons, such as R(OH)ₘ, as an initiator; 2. an alkoxide alone as an initiator, in which a ring-opening polymerization reaction is performed, and controllable polyester polymer compounds can then be obtained by this one-step method. The method for the preparation of the poly(δLH₂) is simple, as well as has high yield and controllability. The catalyst used is simple to prepare, inexpensive, and has good activity.

The molecular weight and molecular weight distribution of the polyester polymer compound poly(δLH₂) can be changed by adjusting the type of raw materials, the amount of the reagents, the reaction temperature, the reaction time, and other conditions. The prepared poly(δLH₂) is a flexible material, which can be completely degraded into monomers. The polyester material has good physicochemical properties, such as high transparency, high molecular weight, high thermal stability, and good ductility.

In the method provided by the present disclosure, cheap and easily available C1 source carbon dioxide and bulk chemical product 1,3-butadiene were used as raw materials to synthesize the six-membered cyclic lactone δLH₂, and then the poly(δLH₂) is synthesized by a ring-opening polymerization reaction, effectively alleviating the greenhouse effect. Monomers δLH₂ can be obtained through the chemical recycling of poly(δLH₂), thus achieving the closed-loop green cycle.

The polyester material prepared in the present disclosure is easy to be modified post-polymerization. Poly(δLH₂) can be modified after polymerization by post-polymerization modification methods, for example, the polymer properties can be regulated by the grafting of olefinic side chains of poly(δLH₂) via the photo-initiated thiol-ene click reaction.

The present disclosure provides a method for the preparation of a disubstituted α,β-saturated six-membered cyclic lactone such as δLH₂. When using a disubstituted α,β-unsaturated six-membered cyclic lactone such as δ-L as a raw material, the disubstituted α,β-saturated six-membered cyclic lactone can be obtained by a selective reduction reaction of conjugated olefins, with a Stryker reagent or a mixture of reagents capable of generating the Stryker reagent in-situ as a catalyst and an organosilane as a hydrogen source. The method for the preparation of δLH₂ has a high yield, simple steps, and a low cost.

The method for preparing poly(δLH₂) and the method for preparing δLH₂ described in the present disclosure have a large application prospect in the field of materials, especially in the field of polymer materials.

### BRIEF DESCRIPTION OF DRAWINGS

FIGS. 1a-1c show possible chain initiation mechanisms of the ^{t}Bu-P₄/BnOH-catalyzed ring-opening polymerization of HL (¹H NMR spectra of equivalent experiments). FIG. 1a. phosphazene ^{t}Bu-P₄; FIG. 1b. BnOH; FIG. 1c. [^{t}Bu-P₄]/[BnOH]=1/1.

FIG. 2 shows a MALDI-TOF of a product according to Embodiment 18.

FIG. 3 shows a MALDI-TOF of a product according to Embodiment 19.

FIGS. 4a-4d show an HL-ROP active polymerization analysis under the catalysis of ^{t}Bu-P₄/BnOH according to Embodiment 20. FIG. 4a represents HL conversion versus time; FIG. 4b represents In([M]/[M]₀); FIG. 4c represents the molecular weight and molecular weight distribution of polyHL versus HL monomer conversion; FIG. 4d represents superimposed GPC curves of polyHL obtained at different polymerization times.

FIG. 5 shows MALDI-TOF of a linear polyHL polymer according to Embodiment 21, indicating that the linear polyHL polymer is end-capped with BnOH.

FIG. 6 shows ¹H NMR of the linear polyHL polymer according to Embodiment 21.

FIG. 7 shows ¹³C NMR of the linear polyHL polymer according to Embodiment 21.

FIG. 8 shows MALDI-TOF of a linear polyHL diols according to Embodiment 21 in which 1,4-BDM serves as an initiator.

FIG. 9 shows TGA and DTG of a linear polyHL according to Embodiment 23.

FIG. 10 shows DSC of the linear polyHL according to Embodiment 23.

FIG. 11 shows HL monomer concentration versus time under different temperature conditions according to Embodiment 24.

FIG. 12 shows Van′t Hoff analysis of HL according to Embodiment 24.

FIG. 13 shows ¹H NMR of the chemical recycled linear polyHL according to Embodiment 25. Bottom part represents NMR spectrum of the linear polyHL (synthesis conditions: [HL]/[^{t}BuP₄]/[BnOH]=50/0.1/1; reaction temperature:−25° C.; solvent: THF, Mₙ=9.2 kg mol⁻¹, Ð=1.08); middle part represents spectrum of chemical recycled colorless oily product with solvent removal; top part represents clean starting monomer HL for ROP.

FIG. 14 shows results of monomer recycling from the polymer through pyrolysis according to Embodiment 26.

FIG. 15 shows a schematic diagram for preparation of a cyclic polymer from CO₂ and butadiene and chemical recycling of the cyclic polymer.

FIG. 16 shows a schematic diagram of the mechanism for synthesizing a cyclic polymer from HL ring-opening polymers.

FIGS. 17a-17d show kinetic data of ^{t}Bu-P₄-catalyzed HL ring-opening polymerization. FIG. 17a represents conversion rate versus time; FIG. 17b represents In([M]0/[M]) versus reaction time; FIG. 17c represents number average molecular weight and molecular weight distribution versus conversion rate; FIG. 17d represents superimposed GPC curves of different embodiments.

FIG. 18 shows MALDI-TOF of a cyclic polyHL product prepared according to Embodiment 44.

FIG. 19 shows ¹H NMR spectrum of a cyclic polyHL polymer (Embodiment 29).

FIG. 20 shows ¹³C NMR spectrum of the cyclic polyHL polymer (Embodiment 29).

FIG. 21 shows MALDI-TOF of a polyHL product when the system is not rigorously dewatered.

FIG. 22 shows ³¹P NMR spectra with different ratios of HL to ^{t}Bu-P₄.

FIG. 23 shows zoomed-in views of ¹H NMR spectra with different ratios of HL to ^{t}Bu-P₄.

FIG. 24 shows general views of ¹H NMR spectra with different ratios of HL to Bu-P₄.

FIG. 25 shows the calculated free energy of ^{t}Bu-P₄ for deprotonation of BnOH, HL, and water.

FIG. 26 shows TGA and DTG of the cyclic polyHL product.

FIG. 27 shows DSC of the cyclic polyHL product.

FIGS. 28a-28d show pressure-sensitive adhesive performance tests of the cyclic polyHL samples. FIG. 28a represents a schematic diagram of a 180° peel test; FIG. 28b represents a cross-sectional SEM image of a film formed from the cyclic polyHL (Embodiment 29); FIG. 28c represents results of 180° peel performance test of the cyclic polyHL samples and three commercial adhesive tapes (see Table 29); FIG. 28d represents the colorlessness and light transmittance of the polyHL film (Embodiment 29).

FIG. 29 shows HNMR spectrum of a polyHL polymer prepared according to Embodiment 30.

FIG. 30 shows HNMR spectrum of a polyHL polymer prepared according to Embodiment 31.

FIG. 31 shows HNMR spectrum of a polyHL polymer prepared according to Embodiment 32.

FIG. 32 shows HNMR spectrum of the products after degrading polyHL according to Embodiment 50.

FIG. 33 shows HNMR spectrum of the products after degrading polyHL according to Embodiment 51.

FIG. 34 shows HNMR spectrum of the products after degrading polyHL according to Embodiment 52.

FIG. 35 shows HNMR spectrum of the products after degrading polyHL according to Embodiment 53.

FIG. 36 shows HNMR spectrum of the products after degrading polyHL according to Embodiment 54.

FIG. 37 shows HNMR spectrum of the products after degrading polyHL according to Embodiment 55.

FIG. 38 shows HNMR spectrum of the products after degrading polyHL according to Embodiment 56.

FIG. 39 shows HNMR spectrum of the products after degrading polyHL according to Embodiment 57.

FIG. 40 shows HNMR spectrum of the products after degrading polyHL according to Embodiment 58.

FIG. 41 shows HNMR spectrum of the products after degrading polyHL according to Embodiment 59.

FIG. 42 shows HNMR spectrum of the products after degrading polyHL according to Embodiment 60.

FIG. 43 shows HNMR spectrum of the products after degrading polyHL according to Embodiment 61.

FIG. 44 shows HNMR spectrum of the products after degrading polyHL according to Embodiment 62.

FIG. 45 shows HNMR spectrum of the products after degrading polyHL according to Embodiment 63.

FIG. 46 shows HNMR spectrum of the products after degrading polyHL according to Embodiment 64.

FIG. 47 shows HNMR spectrum of the products after degrading polyHL according to Embodiment 65.

FIG. 48 shows HNMR spectrum of the products after degrading polyHL according to Embodiment 66.

FIG. 49 shows HNMR spectrum of the products after degrading polyHL according to Embodiment 67.

FIG. 50 shows HNMR spectrum of the products after degrading polyHL according to Embodiment 68.

FIG. 51 shows HNMR spectrum of the products after degrading polyHL according to Embodiment 69.

FIG. 52 shows HNMR spectrum of the products after degrading polyHL according to Embodiment 70.

FIG. 53 shows HNMR spectrum of the products after degrading polyHL according to Embodiment 71.

FIG. 54 shows HNMR spectrum of the products after degrading polyHL according to Embodiment 72.

FIG. 55 shows a superimposed NMH spectrum of starting material, polymer, and degradation product according to Embodiment 73.

FIG. 56 shows GPC data of polyHL_160 prepared according to Embodiment 49.

FIG. 57 shows GPC data of polyHL_319 prepared according to Embodiment 49.

FIG. 58 shows GPC data of polyHL_562 prepared according to Embodiment 49.

FIG. 59 shows the current status and technical synthesis route of poly(δLH₂) of the present disclosure.

FIG. 60 shows HNMR spectrum of δLH₂ prepared according to Embodiment 74.

FIG. 61 shows CNMR spectrum of δLH₂ prepared according to Embodiment 74.

FIG. 62 shows HNMR spectrum of poly(δLH₂) prepared according to Embodiment 81.

FIG. 63 shows GPC of poly(δLH₂) prepared according to Embodiment 82.

FIG. 64 shows superimposed GPC curves of poly(δLH₂) prepared according to Embodiments 83 and 86˜89; where KOMe corresponds to Embodiment 83, NaOMe corresponds to Embodiment 86, KO^{t}Bu corresponds to Embodiment 87, NaO^{t}Bu corresponds to Embodiment 88, and LiO^{t}Bu corresponds to Embodiment 89.

FIG. 65 shows HNMR spectrum of poly(δLH₂) prepared according to Embodiment 83.

FIG. 66 shows CNMR spectrum of poly(δLH₂) prepared according to Embodiment 83.

FIG. 67 shows HSQC spectrum of poly(δLH₂) prepared according to Embodiment 83.

FIG. 68 shows GPCs of poly(δLH₂) prepared according to Embodiments 83-85.

FIG. 69 shows TGA of poly(δLH₂) prepared according to Embodiment 79.

FIG. 70 shows TGA of poly(δLH₂) prepared according to Embodiment 82.

FIG. 71 shows TGA of poly(δLH₂) prepared according to Embodiment 83.

FIG. 72 shows TGA of poly(δLH₂) prepared according to Embodiment 84.

FIG. 73 shows DSC of poly(δLH₂) prepared according to Embodiment 79.

FIG. 74 shows DSC of poly(δLH₂) prepared according to Embodiment 84.

FIG. 75 shows appearance and light transmittance of poly(δLH₂) prepared according to Embodiment 85.

FIG. 76 shows degradation results of Embodiment 91 using the poly(δLH₂) prepared according to Embodiment 81.

FIG. 77 shows GPCs in post-polymerization modification experiments of Application Example 3/Embodiment 92 before and after post-synthetic modification of poly(δLH₂) prepared according to Embodiment 81.

FIG. 78 shows HNMR spectrum of purified poly(δLH₂-SAr) after post-synthetic modification according to Application Example 3/Embodiment 92.

FIG. 79 shows fluorescence emission spectra in post-polymerization modification experiments of Application Example 3/Embodiment 92 before and after post-modification of poly(δLH₂) according to Embodiment 81.

FIG. 80 shows a schematic diagram of a patterning experiment and a mask design in post-synthetic modification experiments according to Application Example 3 or Embodiment 92.

FIG. 81 shows the patterning experiment in post-synthetic modification experiments according to Application Example 3 or Embodiment 92.

FIG. 82 shows a contact angle (55.7°) between water and glass substrate in post-synthetic modification experiments according to Application Example 3 or Embodiment 92, where a scaling factor at the upper left corner is 1 mm, the values of labeled angles are 55.7° (left) and 55.5° (right), respectively.

FIG. 83 shows a hydrophilic and hydrophobic property test of one poly(δLH₂) sample in post-synthetic modification experiments according to Application Example 3 or Embodiment 92, where a contact angle between the poly(δLH₂) sample and water is 72.8±0.08°, a scaling factor at the upper left corner is 0.5 mm, and the values of labeled angles are 72.8° (left) and 73.3° (right), respectively.

FIG. 84 shows a hydrophilic and hydrophobic property test of one poly(δLH₂-SAr) sample in post-synthetic modification experiments according to Application Example 3 or Embodiment 92, where a contact angle between the poly(δLH₂-SAr) sample and water is 93.0±0.99°, a scaling factor at the upper left corner is 1 mm, and the values of labeled angles are 94.4° (left) and 94.6° (right), respectively.

FIG. 85 shows HNMR spectrum of a polyurethane prepared according to Embodiment 93, where the polyurethane is synthesized via a one-pot, two-step method using HL as the monomer.

FIG. 86 shows GPC data of the polyurethane prepared according to Embodiment 93, where the polyurethane is synthesized via a one-pot, two-step method using HL as the monomer.

### DETAILED DESCRIPTION OF THE INVENTION

The following illustrates the embodiments of the present disclosure by means of particular specific examples, and other advantages and efficacies of the present disclosure can be readily appreciated by those skilled in the art according to the contents disclosed herein. The present disclosure can also be implemented or applied through other different specific embodiments, and the details in this specification can also be modified or altered in various ways based on different viewpoints and applications without departing from the spirit of the present disclosure.

The present disclosure overcomes a technical bottleneck of the prior art and reports for the first time a chemical recyclable polymer and a method for synthesizing the same. The polymer may have a monohydroxy-capped or bi-hydroxy-capped structure, its molecular weight may be regulated according to the degree of polymerization, and the carbon dioxide content may reach 28 wt %. The method of the present disclosure enables the reactive polymerization of heterocyclic lactones such as the six-membered lactone 3,6-diethyl-tetrahydro-2h-pyran-2-one (HL). When HL is used as the feedstock, which can be obtained from carbon dioxide and inexpensive bulk chemicals, for example, HL is synthesized from CO₂, H₂, and 1,3-butadiene using a palladium-catalyzed two-step reaction, HL is subjected to anionic ring-opening polymerization (AROP) catalyzed by an organic base (e.g., ^{t}Bu-P₄) to produce the polyester polymer compound according to reaction route 1. The present disclosure also develops a series of catalytic methods to chemically degrade polyester polymer compounds such as polyHL, thereby recycling heterocyclic lactone monomers such as HL monomers.

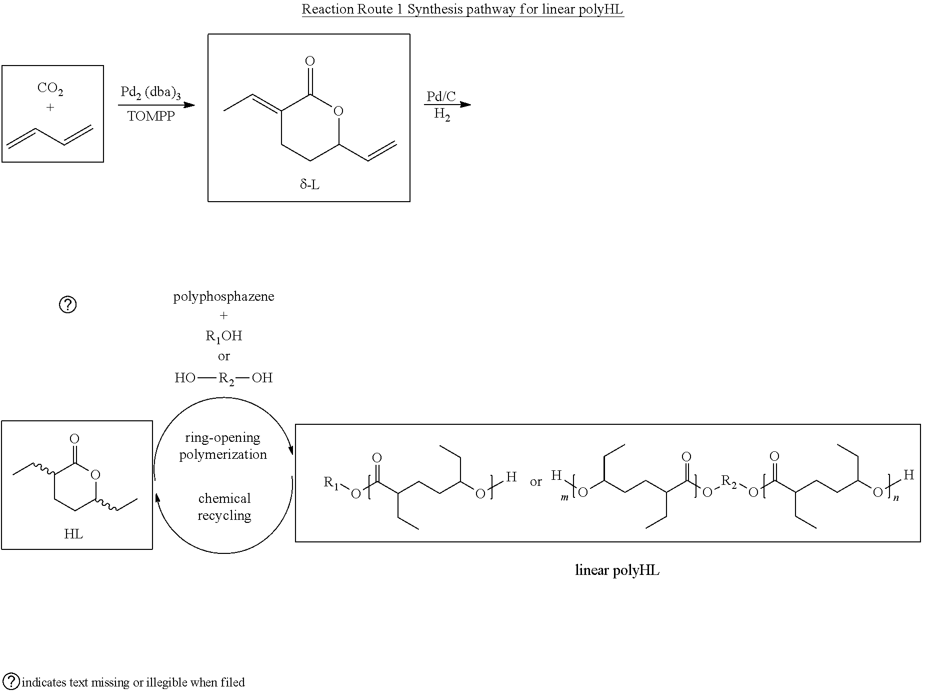

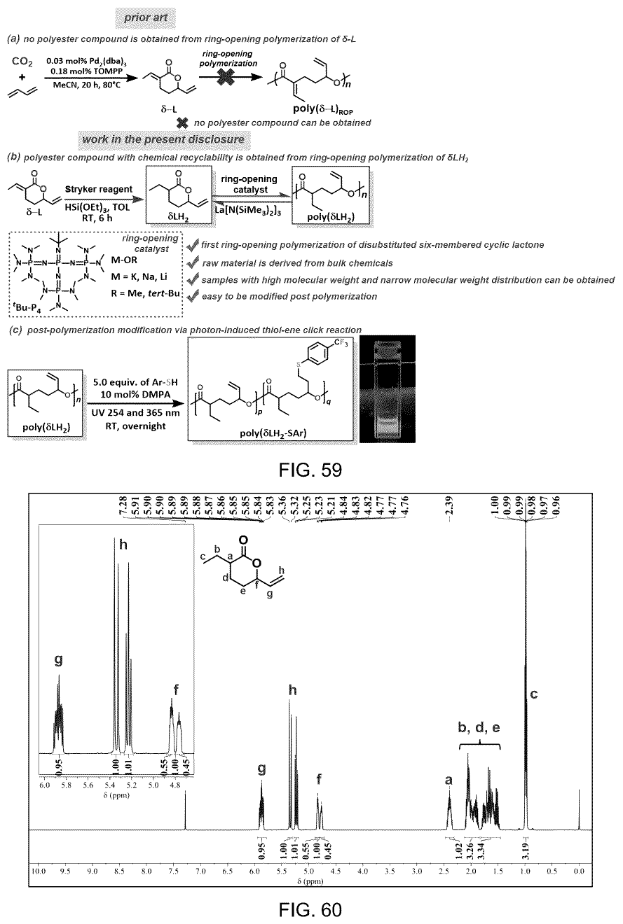

In addition, the present disclosure provides, for the first time, a very efficient multi-step reaction strategy for synthesizing a chemical recyclable CO₂-based polyester i.e., poly(δLH₂) from CO₂ and butadiene. Specifically, δ-L is synthesized from CO₂ and butadiene, and a six-membered cyclic lactone containing terminal olefinic groups in the side chain, i.e., 3-ethyl-6-vinyl-tetrahydro-2H-pyran-2-one (δLH₂), is prepared by selectively reduction of conjugated olefins of δ-L. The CO₂-based polyester poly(δLH₂) is obtained for the first time by the ring-opening polymerization of such six-membered cyclic lactones δLH₂ with two substituents. The polyester features a high CO₂ usage rate (e.g., preferably up to 29 wt %), good transparency, high molecular weight, and good thermal stability. The polyester can be completely degraded back to monomers with the participation of a catalyst. Furthermore, due to the presence of terminal olefinic side chains in the repeating units of the polyester, it is easy to perform post-synthesis modification on the polymer poly(δLH₂). For example, the polymer properties can be regulated by grafting onto the olefinic side chains of poly(δLH₂) through a photoinduced thiol-ene click reaction, as disclosed in the present disclosure. The synthetic pathway of the CO₂-based polyester poly(δLH₂) of the present disclosure is shown as reaction route 2. The current status and technical route for synthesizing CO₂-based polyester poly(δLH₂) are shown in FIG. 59.

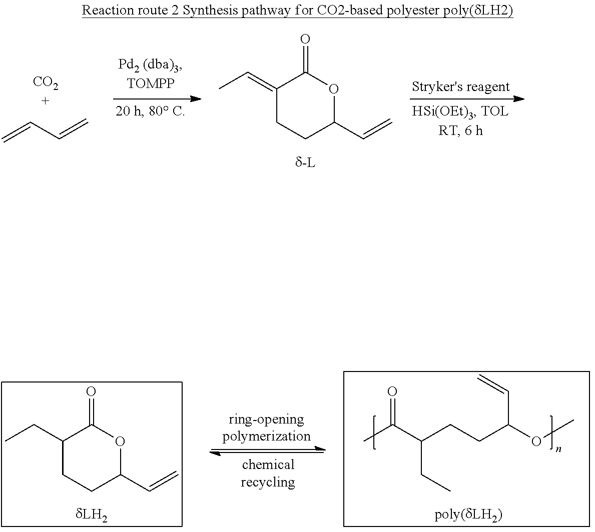

Furthermore, the present disclosure provides, for the first time, a monomer-recyclable cyclic polymer and a method for synthesizing the same, where the cyclic polymer is prepared from heterocyclic lactones through anionic ring-opening polymerization. The synthesis method can regulate the degree of polymerization of the cyclic polymer, thereby controlling the Mₙ (number average molecular weight) and Ð (molecular weight distribution) of the polymer. The present disclosure also provides a series of methods for recycling monomers of the cyclic polymer. The synthetic method described in the present disclosure allows for the preparation of a cyclic polymer using simple and readily available feedstocks. For example, heterocyclic lactone 3,6-diethyl-tetrahydro-2H-pyran-2-one (HL) can be obtained from carbon dioxide and inexpensive bulk chemicals. Specifically, an unsaturated lactone δ-lactone containing two carbon-carbon double bonds is synthesized from CO₂ and 1,3-butadiene, and then δ-lactone reacts with hydrogen under the catalysis of transition metal to produce HL. In other words, HL is synthesized from CO₂, H₂, and 1,3-butadiene using a palladium-catalyzed two-step reaction. As shown in the reaction route 3, the produced HL is then subjected to anionic ring-opening polymerization (AROP) catalyzed by organic bases (e.g., ^{t}Bu-P₄) to obtain the cyclic polymer polyHL, which can be cracked via the catalytic method of the present disclosure to obtain the monomer HL.

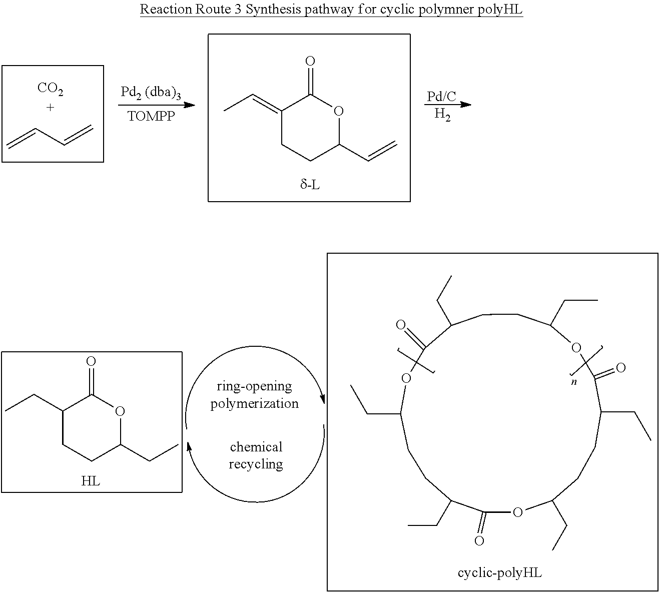

The present disclosure provides a polyester polymer compound, where the polyester polymer compound comprises repeating units shown as formula 4 below:

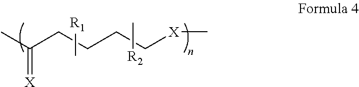

where R₁ and R₂ may be the same or different, R₁ and R₂ exist on a six-membered heterocycle simultaneously, and locate at any two of the four methylene carbons.

R₁, R₂ are each independently selected from any one of hydrogen, halogen, alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group, where the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups;
and the halogen is selected from fluorine, chlorine, bromine, and iodine.

The alkyl may be linear, branched, or cyclic; further, the alkyl may be C1 to C20 alkyl or C1 to C10 alkyl. Examples include methyl, ethyl, propyl, butyl, pentyl, hexyl, heptyl, octyl, nonyl, and decyl, with ethyl being preferred.

The substituted alkyl may be substituted linear alkyl, substituted branched alkyl, or substituted cyclic alkyl.

The alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide group, sulfonamide group, morpholinyl, and piperazinyl. Further, the alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, hydroxyl, nitro, halo, carboxyl, C1-C10 alkyl, alkoxyl, amine, cycloalkoxyl, cycloalkylamine group, C2-C12 alkenyl, C2-C12 alkynyl, C3-C12 cycloalkyl, benzyl, alkyl carbonyl, C2-C12 alkenyl carbonyl, C3-C12 cycloalkyl carbonyl, phenyl carbonyl, benzyl carbonyl, alkoxyl carbonyl, esteryl, sulfinyl, sulfonyl, sulfinamide group, sulfonamide group, morpholinyl, and piperazinyl.

Preferably, R₁ is C1-C20 alkyl, and R₂ is C1-C20 alkyl; further preferably, R₁ is C1-C10 alkyl, and R₂ is C1-C10 alkyl. R₁ and R₂ may be C1, C2, C3, C4, C5, C6, C7, C8, C9, C10 alkyl, respectively. Further preferably, R₁ and R₂ are ethyl.

X is a heteroatom, and is selected from O, S, N, P, and the like; preferably, X is O.

n is a positive integer greater than or equal to 1, for example, n may be in a range of 1-100, 100-1000, 1000-5000, 5000-10000, 10000-15000, 15000-20000, 20000-30000, 30000-40000, 40000-50000, 50000-100000, 100000-200000, etc.

As a preference, the polyester polymer compound is a CO₂-based polyester poly(δLH₂), where the CO₂-based polyester poly(δLH₂) comprises repeating units shown as formula 6 below:

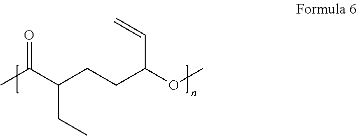

where n is a positive integer greater than or equal to 1.

The polyester polymer compound comprises a linear, cyclic, star-shaped topology.

The polyester polymer compound with a linear structure has a structure shown as formula 2:

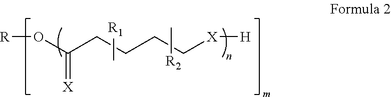

R₁, R₂, X, n, m are defined as described in the compound of formula 4 above.

R is selected from any one of alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol.

Preferably, R may represent an aromatic group comprising a phenyl, a naphthyl, an anthryl, a phenanthrenyl, a pyrenyl, a benzo (a) pyrene and derivatives thereof, etc. Specifically, R may be selected from phenyl, benzyl, homo-tribenzyl, ortho-dibenzyl, meta-dibenzyl, para-dibenzyl, 1,2,3-tribenzyl, 1,2,4-tribenzyl, 1,2,3,4-tetrabenzyl, 1,2,3,5-tetrabenzyl, 1,2,4,5-tetrabenzyl, pentabenzyl, hexabenzyl, phenylethyl, homo-triethylphenyl, ortho-diethylphenyl, meta-diethylphenyl, para-diethylphenyl, 1,2,3-triethylphenyl, 1,2,4-triethylphenyl, 1,2,3,4-tetraethylphenyl, 1,2,3,5-tetraethylphenyl, 1,2,4,5-tetraethylphenyl, pentaethylphenyl, hexaethylphenyl, benzylpropyl, homo-tris-n-propylphenyl, ortho-di-n-propylphenyl, meta-di-n-propylphenyl, para-di-n-propylphenyl, 1,2,3-tri-n-propylphenyl, 1,2,4-tri-n-propylphenyl, 1,2,3,4-tetra-n-propylphenyl, 1,2,3,5-tetra-n-propylphenyl, 1,2,4,5-tetra-n-propylphenyl, penta-n-propylphenyl, hexa-n-propylphenyl, isopropylphenyl, homo-tri-isopropylphenyl, ortho-di-isopropylphenyl, meta-di-isopropylphenyl, para-di-isopropylphenyl, 1,2,3-tri-isopropylphenyl, 1,2,4-tri-isopropylphenyl, 1,2,3,4-tetra-isopropylphenyl, 1,2,3,5-tetra-isopropylphenyl, 1,2,4,5-tetra-isopropylphenyl, penta-isopropylphenyl, hexa-isopropylphenyl, n-butylphenyl, isobutylphenyl, tert-butylphenyl, ortho-di-n-butylphenyl, meta-di-n-butylphenyl, para-di-n-butylphenyl, ortho-di-isobutylphenyl, meta-di-isobutylphenyl, para-di-isobutylphenyl, ortho-di-tert-butylphenyl, meta-di-tert-butylphenyl, and para-di-tert-butylphenyl.

Preferably, R may also be a straight or branched alkane; more preferably, R may be a linear or branched alkane with a carbon number ranging from 1 to 10 or a alkyl substituent containing an unsaturated bond, where the unsaturated bond refers to a carbon-carbon double bond in which SP2 hybridization occurs and a carbon-carbon triple bond in which SP hybridization occurs.

Preferably, R may also be a polymer group with a molecular weight no greater than 100,000 g/mol and containing repeating units of polyvinyl alcohol and/or polyethylene glycol.

Further preferably, R is benzyl, para-dibenzyl, (CH₂)₃, or (CH₂)₄.

Further preferably, when the polyester polymer compound is a CO₂-based polyester poly(δLH₂), the CO₂-based polyester poly(δLH₂) has a structure shown as formula 7:

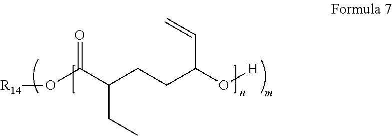

where R₁₄ is selected from C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol.
n is a positive integer no less than 1; and
m is a positive integer no less than 1.

Specifically, R₁₄ may represent an aromatic group comprising a phenyl, a naphthyl, an anthryl, a phenanthrenyl, a pyrenyl, a benzo (a) pyrene and derivatives thereof, etc. Specifically, R₁₄ may be selected from phenyl, benzyl, homo-tribenzyl, ortho-dibenzyl, meta-dibenzyl, para-dibenzyl, 1,2,3-tribenzyl, 1,2,4-tribenzyl, 1,2,3,4-tetrabenzyl, 1,2,3,5-tetrabenzyl, 1,2,4,5-tetrabenzyl, pentabenzyl, hexabenzyl, phenylethyl, homo-triethylphenyl, ortho-diethylphenyl, meta-diethylphenyl, para-diethylphenyl, 1,2,3-triethylphenyl, 1,2,4-triethylphenyl, 1,2,3,4-tetraethylphenyl, 1,2,3,5-tetraethylphenyl, 1,2,4,5-tetraethylphenyl, pentaethylphenyl, hexaethylphenyl, benzylpropyl, homo-tris-n-propylphenyl, ortho-di-n-propylphenyl, meta-di-n-propylphenyl, para-di-n-propylphenyl, 1,2,3-tri-n-propylphenyl, 1,2,4-tri-n-propylphenyl, 1,2,3,4-tetra-n-propylphenyl, 1,2,3,5-tetra-n-propylphenyl, 1,2,4,5-tetra-n-propylphenyl, penta-n-propylphenyl, hexa-n-propylphenyl, isopropylphenyl, homo-tri-isopropylphenyl, ortho-di-isopropylphenyl, meta-di-isopropylphenyl, para-di-isopropylphenyl, 1,2,3-tri-isopropylphenyl, 1,2,4-tri-isopropylphenyl, 1,2,3,4-tetra-isopropylphenyl, 1,2,3,5-tetra-isopropylphenyl, 1,2,4,5-tetra-isopropylphenyl, penta-isopropylphenyl, hexa-isopropylphenyl, n-butylphenyl, isobutylphenyl, tert-butylphenyl, ortho-di-n-butylphenyl, meta-di-n-butylphenyl, para-di-n-butylphenyl, ortho-di-isobutylphenyl, meta-di-isobutylphenyl, para-di-isobutylphenyl, ortho-di-tert-butylphenyl, meta-di-tert-butylphenyl, and para-di-tert-butylphenyl.

Specifically, R₁₄ may also be a linear or branched alkane; more preferably, R 14 may be a linear or branched alkane with a carbon number ranging from 1 to 10 or a alkyl substituent containing an unsaturated bond, where the unsaturated bond refers to a carbon-carbon double bond in which SP2 hybridization occurs and a carbon-carbon triple bond in which SP hybridization occurs.

Specifically, R₁₄ may also be a polymer group with a molecular weight no greater than 100,000 g/mol and containing repeating units of polyvinyl alcohol and/or polyethylene glycol.

Preferably, R₁₄ is benzyl, para-dibenzyl, (CH₂)₃, or (CH₂)₄.

Specifically, n represents the number of repeating units and n is a positive integer no less than 1, for example, n may be in a range of 1-100, 100-1000, 1000-5000, 5000-10000, 10000-15000, 15000-20000, 20000-30000, 30000-40000, 40000-50000, 50000-100000, 100000-200000, etc.

Specifically, m is the degree of branching, and m is a positive integer no less than 1. For example, 1≤m≤10, 10≤m≤20; preferably, 1≤m≤10. m may also represent the functionality of an initiator R₁₄ (OH)ₘ and/or (MO)ₘR₁₄.

The polyester polymer compound with a cyclic structure has a structure shown as formula 3:

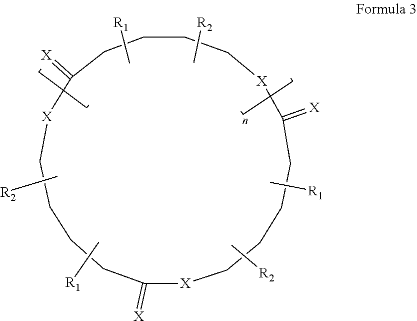

R₁, R₂, X, n are defined as described in the compound of formula 4 above.

The present disclosure provides a method for synthesizing a polyester polymer compound, where the method is selected from the following:
A) when the polyester polymer compound has a linear structure, the method comprises: conducting a reaction on heterocyclic lactones shown as formula 1 to obtain the polyester polymer compound shown as formula 2 under the catalysis of organic base and the initiation of R(OH)ₘ capable of supplying an active proton; where the reaction is represented as Reaction formula I:

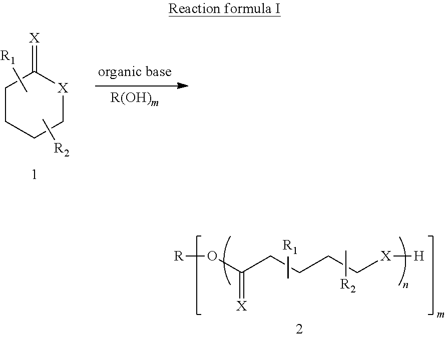

B) when the polyester polymer compound has a cyclic structure, the method comprises: conducting a reaction on heterocyclic lactones shown as formula 1 to obtain the polyester polymer compound shown as formula 3 under the catalysis of organic base; where the reaction is represented as Reaction formula II:

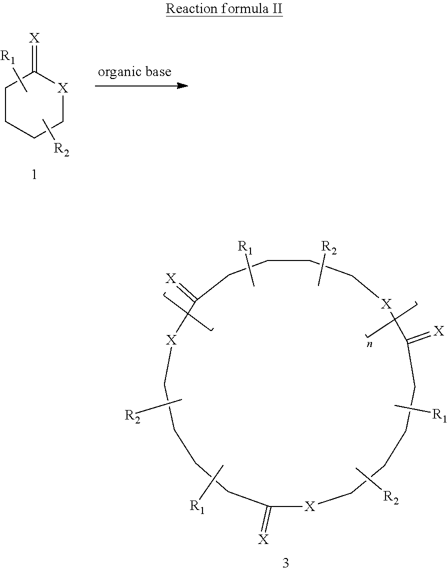

In A) and/or B), R₁, R₂ may be the same or different, and R₁, R₂ exist on a six-membered heterocycle simultaneously, and locate at any two of the four methylene carbons.

In A) and/or B), R₁ and R₂ are each independently selected from hydrogen, halogen, alkyl (linear alkyl, branched alkyl, cycloalkyl), substituted alkyl (linear alkyl, branched alkyl, cycloalkyl), alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group. The monocyclic aromatic group includes phenyl, aza-aromatic group, thio-aromatic group, and oxa-aromatic group, and the polycyclic aromatic group and polyheterocyclic aromatic group refer to a group comprising two or more monocyclic aromatic groups.

The halogen is selected from fluorine, chlorine, bromine, and iodine.

The alkyl may be linear, branched, or cyclic; further, the alkyl may be C1 to C20 alkyl or C1 to C10 alkyl. Examples include methyl, ethyl, propyl, butyl, pentyl, hexyl, heptyl, octyl, nonyl, and decyl, with ethyl being preferred.

The substituted alkyl may be substituted linear alkyl, substituted branched alkyl, or substituted cyclic alkyl.

The alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide, sulfonamide group, morpholinyl, and piperazinyl. Further, the alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, hydroxyl, nitro, halo, carboxyl, C1-C10 alkyl, alkoxyl, amine, cycloalkoxyl, cycloalkylamine group, C2-C12 alkenyl, C2-C12 alkynyl, C3-C12 cycloalkyl, benzyl, alkyl carbonyl, C2-C12 alkenyl carbonyl, C3-C12 cycloalkyl carbonyl, phenyl carbonyl, benzyl carbonyl, alkoxyl carbonyl, esteryl, sulfinyl, sulfonyl, sulfinamide group, sulfonamide group, morpholinyl, and piperazinyl.

Preferably, R₁ is C1-C20 alkyl, and R₂ is C1-C20 alkyl; further preferably, R₁ is C1-C10 alkyl, and R₂ is C1-C10 alkyl. R₁ and R₂ may be C1, C2, C3, C4, C5, C6, C7, C8, C9, C10 alkyl, respectively. Further preferably, R₁ and R₂ are ethyl.

In reaction formula I or II, X is a heteroatom, and is selected from O, S, N, P, etc.; preferably, X is O.

In the reaction formula I or II, n represents the number of repeating units, and n is a positive integer no less than 1; for example, n may be in a range of 1-100, 100-1000, 1000-5000, 5000-10000, 10000-15000, 15000-20000, 20000-30000, 30000-40000, 40000-50000, 50000-100000, 100000-200000, etc.

In the reaction formula I, m is a degree of branching, and m is a positive integer no less than 1. For example, 1≤m≤10, 10≤m≤20; preferably, 1≤m≤10. m may also represent the functionality of the initiator ROH.

In reaction formula I or II, the organic base may be a sterically hindered base or a non-nucleophilic base.

In A), when the polyester polymer compound is a CO₂-based polyester poly(δLH₂), the method comprises: obtaining the CO₂-based polyester poly(δLH₂) shown as formula 7 by synthesis using δLH₂ shown as formula 8 under the catalysis of an organic base and the initiation of initiator R₁₄ (OH)ₘ and/or (MO)ₘR₁₄ capable of providing active protons, where the reaction is represented as reaction formula III:

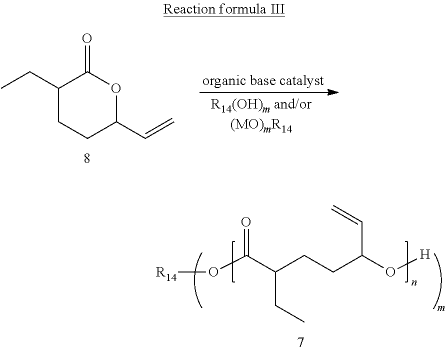

and R₁₄ is defined as described above.

The method described in the present disclosure may be conducted with both the catalyst and the initiator present simultaneously, or with only the initiator present.

In a preferred embodiment, the organic base is an amine compound or a nitrogen-containing heterocyclic compound; where the amine compound has a structure of the following formula:

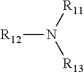

Ammonium salt has a structure of R₁₁R₁₂R₁₃N⁺H, where R₁₁, R₁₂, and R₁₃ each independently is a hydrogen (H), C1-C20 alkyl, C5-C20 cycloalkyl, or C7-C20 alkylaryl. These groups each optionally comprises one or more heteroatoms (e.g., oxygen, phosphorus, or sulphur atom) and/or substituents. A ring may exist between R₁₁ and R₁₂, R₁₂ and R₁₃, and/or R₁₁ and R₁₃, where the ring may comprise a heteroatom.

Preferably, the organic base is selected from one or more of a phosphazene, a compound containing a guanidine group, and a compound containing an amidino group. In some embodiments, the organic base is selected from ^{t}Bu-P₁, ^{t}Bu-P₂, ^{t}Bu-P₄, 1,5,7-triazabicyclo[4.4.0]deca-5-ene (TBD), 1,8-diazabicycloundec-7-ene (DBU), diethylamine, dimethylamine, triethylamine, N,N-diisopropylethylamine, N-methyl morpholine, n-octylamine, tri-n-butylamine, laurylamine, stearylamine, tetra-propylammonium hydroxide (TPAOH), tetra-butylammonium hydroxide (TBAOH), sodium alcoholate or potassium alcoholate of C1˜C5, triethanolamine, choline, N-methylmorpholine, pyridine, dimethylaminopyridine, N, N′-dihydroxyethyl ethylenediamine, β-hydroxyethyl ethylenediamine, N-(2-hydroxyethyl)ethylenediamine, N, N,N′,N′-tetrahydroxyethyl ethylenediamine, N-hydroxyethyl propylenediamine, trimethylhydroxyethyl propylenediamine, and N,N′-bis-(2-hydroxyethyl)-1,3-propylenediamine.

Preferably, the catalyst is one or more of phosphazene and TBD. Further preferably, the phosphazene is a strong Lewis base having the structure of (R₂N)₂₋P═N. Further preferably, the phosphazene has a structure shown as formula 5:

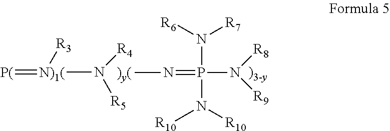

In formula 5, R₃—R₁₀ are independently selected from alkyl. preferably C1-C10 alkyl, such as C1 (methyl), C2 (ethyl), C3 (propyl, isopropyl), C4 (butyl, tert-butyl), C5, C6, C7, C8, C9, C10 alkyl, respectively.

y is a positive integer no less than 1. Preferably, 1≤y≤3.

Preferably, the phosphazene is selected from ^{t}Bu-P₁, ^{t}Bu-P₂, ^{t}Bu-P₄, with the structures shown below, respectively:

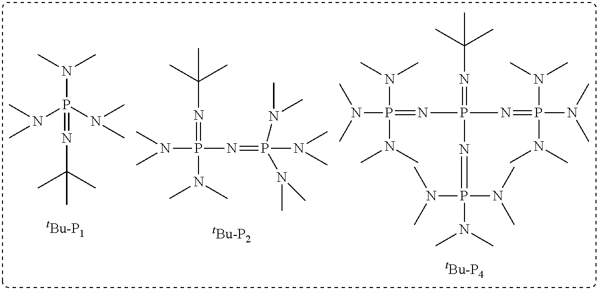

In reaction formula I, the initiator is capable of providing active protons, specifically is an alcohol; preferably, an alkoxide.

The alkoxide is R(OH)ₘ, where R is selected from alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and a polymer group with a molecular weight no greater than 100,000 g/mol and containing repeating units of polyvinyl alcohol and/or polyethylene glycol. m may also represent the functionality of the initiator, and m is a positive integer no less than 1; for example, 1≤m≤10, 10≤m≤20; preferably, 1≤m≤10.

When using only the initiator without adding a catalyst, preferably, in reaction formula III, the initiator is (MO)ₘR₁₄, or a mixture of R₁₄ (OH)ₘ and (MO)ₘR₁₄, where the mixture comprises one or more R₁₄ (OH)ₘ as well as one or more (MO)ₘR₁₄. In some preferred embodiments, the mixture comprises one R₁₄ (OH)ₘ and one (MO)ₘR₁₄. CO₂-based polyesters poly(δLH₂), for example, can also be obtained under the catalysis of an organic base without an initiator. Specifically, in reaction formula III, the initiator is R₁₄ (OH)ₘ and/or (MO)ₘR₁₄ capable of providing active protons, where R₁₄ is selected from alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and a polymer group with a molecular weight no greater than 100,000 g/mol and containing repeating units of polyvinyl alcohol and/or polyethylene glycol.

Preferably, R may represent an aromatic group comprising a phenyl, a naphthyl, an anthryl, a phenanthrenyl, a pyrenyl, a benzo (a) pyrene and derivatives thereof, etc. Specifically, R may be selected from phenyl, benzyl, homo-tribenzyl, ortho-dibenzyl, meta-dibenzyl, para-dibenzyl, 1,2,3-tribenzyl, 1,2,4-tribenzyl, 1,2,3,4-tetrabenzyl, 1,2,3,5-tetrabenzyl, 1,2,4,5-tetrabenzyl, pentabenzyl, hexabenzyl, phenylethyl, homo-triethylphenyl, ortho-diethylphenyl, meta-diethylphenyl, para-diethylphenyl, 1,2,3-triethylphenyl, 1,2,4-triethylphenyl, 1,2,3,4-tetraethylphenyl, 1,2,3,5-tetraethylphenyl, 1,2,4,5-tetraethylphenyl, pentaethylphenyl, hexaethylphenyl, benzylpropyl, homo-tris-n-propylphenyl, ortho-di-n-propylphenyl, meta-di-n-propylphenyl, para-di-n-propylphenyl, 1,2,3-tri-n-propylphenyl, 1,2,4-tri-n-propylphenyl, 1,2,3,4-tetra-n-propylphenyl, 1,2,3,5-tetra-n-propylphenyl, 1,2,4,5-tetra-n-propylphenyl, penta-n-propylphenyl, hexa-n-propylphenyl, isopropylphenyl, homo-tri-isopropylphenyl, ortho-di-isopropylphenyl, meta-di-isopropylphenyl, para-di-isopropylphenyl, 1,2,3-tri-isopropylphenyl, 1,2,4-tri-isopropylphenyl, 1,2,3,4-tetra-isopropylphenyl, 1,2,3,5-tetra-isopropylphenyl, 1,2,4,5-tetra-isopropylphenyl, penta-isopropylphenyl, hexa-isopropylphenyl, n-butylphenyl, isobutylphenyl, tert-butylphenyl, ortho-di-n-butylphenyl, meta-di-n-butylphenyl, para-di-n-butylphenyl, ortho-di-isobutylphenyl, meta-di-isobutylphenyl, para-di-isobutylphenyl, ortho-di-tert-butylphenyl, meta-di-tert-butylphenyl, and para-di-tert-butylphenyl.

Preferably, R may also be a linear or branched alkane; more preferably, R may be a linear or branched alkane with a carbon number ranging from 1 to 10 or a alkyl substituent containing an unsaturated bond, where the unsaturated bond refers to a carbon-carbon double bond in which SP2 hybridization occurs and a carbon-carbon triple bond in which SP hybridization occurs.

Preferably, R may also be a polymer group with a molecular weight no greater than 100,000 g/mol and containing repeating units of polyvinyl alcohol and/or polyethylene glycol.

Further preferably, R is benzyl, para-dibenzyl, (CH₂)₃, or (CH₂)₄, that is, the initiator is selected from phenyl methanol (BnOH), 1,4-benzenedimethanol (1,4-BDM), 1,3-propylene glycol, or 1,4-butanediol.

Preferably, R₁₄ may be a linear or branched alkane with a carbon number ranging from 1 to 10, an alkyl substituent containing an unsaturated bond, or an aromatic group, where the unsaturated bond refers to a carbon-carbon double bond in which SP2 hybridization occurs and a carbon-carbon triple bond in which SP hybridization occurs.

Preferably, R₁₄ may represent a C1-C10 alkyl or an aromatic group comprising a phenyl, a naphthyl, an anthryl, a phenanthrenyl, a pyrenyl, a benzo (a) pyrene and derivatives thereof, etc. Specifically, R₁₄ may be selected from methyl, ethyl, propyl, isopropyl, phenyl, benzyl, homo-tribenzyl, ortho-dibenzyl, meta-dibenzyl, para-dibenzyl, 1,2,3-tribenzyl, 1,2,4-tribenzyl, 1,2,3,4-tetrabenzyl, 1,2,3,5-tetrabenzyl, 1,2,4,5-tetrabenzyl, pentabenzyl, hexabenzyl, phenylethyl, homo-triethylphenyl, ortho-diethylphenyl, meta-diethylphenyl, para-diethylphenyl, 1,2,3-triethylphenyl, 1,2,4-triethylphenyl, 1,2,3,4-tetraethylphenyl, 1,2,3,5-tetraethylphenyl, 1,2,4,5-tetraethylphenyl, pentaethylphenyl, hexaethylphenyl, benzylpropyl, homo-tris-n-propylphenyl, ortho-di-n-propylphenyl, meta-di-n-propylphenyl, para-di-n-propylphenyl, 1,2,3-tri-n-propylphenyl, 1,2,4-tri-n-propylphenyl, 1,2,3,4-tetra-n-propylphenyl, 1,2,3,5-tetra-n-propylphenyl, 1,2,4,5-tetra-n-propylphenyl, penta-n-propylphenyl, hexa-n-propylphenyl, isopropylphenyl, homo-tri-isopropylphenyl, ortho-di-isopropylphenyl, meta-di-isopropylphenyl, para-di-isopropylphenyl, 1,2,3-tri-isopropylphenyl, 1,2,4-tri-isopropylphenyl, 1,2,3,4-tetra-isopropylphenyl, 1,2,3,5-tetra-isopropylphenyl, 1,2,4,5-tetra-isopropylphenyl, penta-isopropylphenyl, hexa-isopropylphenyl, n-butylphenyl, isobutylphenyl, tert-butylphenyl, ortho-di-n-butylphenyl, meta-di-n-butylphenyl, para-di-n-butylphenyl, ortho-di-isobutylphenyl, meta-di-isobutylphenyl, para-di-isobutylphenyl, ortho-di-tert-butylphenyl, meta-di-tert-butylphenyl, and para-di-tert-butylphenyl.

Further preferably, R₁₄ is benzyl or methyl.

Further preferably, M is K, Na, Li, Rb, or H.

Further preferably, the initiator is selected from phenyl methanol (BnOH), potassium methoxide (KOMe), sodium methoxide (NaOMe), potassium tert-butoxide (KO^{t}Bu), sodium tert-butoxide (NaO^{t}Bu), or lithium tert-butoxide (LiO^{t}Bu).

In reaction formula III, n represents the number of repeating units, and n is a positive integer no less than 1; for example, n may be in a range of 1-100, 100-1000, 1000-5000, 5000-10000, 10000-15000, 15000-20000, 20000-30000, 30000-40000, 40000-50000, 50000-100000, 100000-200000, etc.

In the reaction formula III, m is the degree of branching, and m is a positive integer no less than 1. For example, 1≤m≤10, 10≤m≤20; preferably, 1≤m≤10. m may also represent the functionality of an initiator R₁₄ (OH)ₘ and/or (MO)ₘR₁₄.

In the method described in the present disclosure, in reaction formula I, a molar ratio of the compound of formula 1, the catalyst, and the initiator is (5-200):(0.01-5): 1; preferably, (25-100):(0.1-1):1. For example, the molar ratio is (25-30):(0.1-1):1, (35-40):(0.1-1):1, (45-50):(0.1-1):1, (55-60):(0.1-1):1, (65-70):(0.1-1):1, (75-80):(0.1-1): 1, (85-90):(0.1-1):1, (95-100):(0.1-1):1, (25-100):(0.1-0.2):1, (25-100):(0.2-0.3):1, (25-100):(0.3-0.4):1, (25-100):(0.4-0.5):1, (25-100):(0.5-0.6):1, (25-100):(0.6-0.7):1, (25-100):(0.7-0.8):1, (25-100):(0.8-0.9):1, or (25-100):(0.9-1):1; preferably, 40:0.5:1, 50:0.5:1, 50:1:1, 50:0.25:1, 50:0.2:1, 50:0.1:1, 25:0.1:1, or 100:0.2:1; further preferably, 50:0.5:1, 50:1:1, 50:0.25:1, 50:0.2:1, 50:0.1:1, 25:0.1:1, or 100:0.2:1.

Preferably, in reaction formula III, a molar ratio of the compound of formula 8, the catalyst, and the initiator is (5-200):(0-5):(0-1); where the catalyst and the initiator are not simultaneously 0. For example, the molar ratio is (25-200):(0-1):(0-1); preferably, (50-200):(0-1):(0-1), such as (50-55):(0-1):(0-1), (55-60):(0-1):(0-1), (65-70):(0-1):(0-1), (75-80):(0-1):(0-1), (85-90):(0-1):(0-1), (95-100):(0-0.1):(0-1), (100-110):(0.1-0.2):(0-1), (110-120):(0.2-0.3):(0-1), (120-130):(0.3-0.4):(0-1), (130-140):(0.4-0.5):(0-1), (140-150):(0.5-0.6):(0-1), (150-160):(0.6-0.7):(0-1), (160-170):(0.7-0.8):(0-1), (170-180):(0.8-0.9):(0-1), (180-190):(0.9-1):(0-1), or (190-200):(0.9-1):(0-1); further preferably, 50:0.1:0, 50:0.1:1, 50:0.2:1, 50:0.5:1, 50:1:1, 50:0:1, 100:0:1, or 200:0:1.

In the method described in the present disclosure, in reaction formula II, a molar ratio of the compound of formula 1 to the catalyst is (10-500):(0.01-5); preferably, (20-40):(0.1-4), further preferably, (30-350):(0.3-3), such as (40-300):(0.8-1.5). For example, the ratio is (50-260):1; preferably, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, 240:1, 250:1, or 260:1.

The reaction shown in the reaction formula I, II, or III can be carried out under solvent-free conditions (i.e., intrinsic conditions) or solvent conditions.

When the reaction is carried out under solvent conditions, the solvent is selected from: tetrahydrofuran (THF), TBD, benzene, toluene, xylene, dichlorobenzene, mesitylene, dichloromethane, chloroform, 1,2-dichloroethane, tetrahydropyrrole, tetrapyran, hexahydropyridine, ethyl acetate, ethyl ether, dimethyl ether, methyl ethyl ether, n-hexane, cyclohexane, cyclopentane, acetonitrile, dioxane, N,N-dimethylformamide, dimethyl sulphoxide, etc.; preferably, tetrahydrofuran (THF) and/or TBD. Further preferably, the solvent is THF.

In the method described in the present disclosure, there is no particular limitation on the initial concentration [M]₀ of the polyester polymer compound shown as formula 1, as long as the preparation of the polyester polymer compound can be achieved.

In some embodiments, in reaction formula I, the initial concentration [M]₀ of the polyester polymer compound of formula 1 is 1, 2, 3, 4, 5, 6, 7, 8, 9, 10, 11, 12, 13, 14, 15, 16, 17, 18, 19, or 20 mol L⁻¹; preferably, 3.0-10.0 mol L⁻¹. further preferably, 4.0-8.0 mol L⁻¹. For example, the initial concentration is 5.0-6.3 mol L⁻¹; preferably, 5.0, 5.3, or 6. 3 mol L⁻¹.

In some embodiments, in reaction formula II, the initial concentration [M]₀ of the heterocyclic lactone of formula 1 is 0.1-20 mol L⁻¹, for example, 1, 2, 3, 4, 5, 6, 7, 8, 9, 10, 11, 12, 13, 14, 15, 16, 17, 18, 19, or 20 mol L⁻¹. Preferably, the initial concentration is 0.4-16 mol L⁻¹; further preferably, 0.6-12 mol L⁻¹; more further preferably, 1.2-8 mol L⁻¹, such as 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0 mol L⁻¹. In some preferred embodiments, the initial concentration [M]₀ of the heterocyclic lactone is 3-6 mol L⁻¹. In some other preferred embodiments, the initial concentration [M]₀ of the heterocyclic lactone is 4-5 mol L⁻¹. In yet other preferred embodiments, the initial concentration [M]₀ of the heterocyclic lactone is 4.0 mol L⁻¹.

In some embodiments, in reaction formula III, the initial concentration of the compound of formula 8 [δLH₂]₀ in the solvent is 1-7 M; preferably, 3-7 M; further preferably, 5-6 M; more further preferably, 5.5-6 M; such as 5.62 or 5.82 M.

In the method described in the present disclosure, there is no particular limitation on the temperature of the reaction, as long as the preparation of the polyester polymer compound can be achieved.

In some embodiments, in reaction formula I or III, the reaction temperature is −100˜200° C., such as −100˜180° C., −80˜200° C., −100˜150° C., −100˜130° C., −100˜120° C., −100˜100° C., −80˜80° C., −70˜70° C., −60˜60° C., −50˜50° C., −50˜60° C., −40˜40° C., −30˜30° C., −20˜ 20° C., or −10˜10° C. Preferably, the reaction temperature is −50˜60° C. Further preferably, the reaction temperature is −25 to 45° C., for example, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C. More further preferably, the reaction temperature ranges from −25 to 9° C., such as −25° C.

In some embodiments, in reaction formula II, the reaction temperature is −100 to 220° C., for example, −100˜180° C., −80˜200° C., −100˜150° C., −100˜130° C., −100˜120° C., −100˜100° C., −80˜80° C., −70˜70° C., −60˜60° C., −50˜50° C., −50˜60° C., −40˜40° C., −30˜30° C., −20˜20° C., or −10˜10° C. Preferably, the reaction temperature is −80˜80° C. More preferably, the reaction temperature is −50˜60° C. Still more preferably, the reaction temperature is −50˜30° C., for example, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° C. In some preferred embodiments, the reaction temperature is −30˜−10° C. In some preferred embodiments, the reaction temperature is −25° C.

In the method described in the present disclosure, there is no particular limitation on the duration of the reaction, as long as the preparation of the polyester polymer compound can be achieved.

In some embodiments, in reaction formula I, the reaction time is 10 s-36 h; for example, 10 s-360 h, 20 s-340 h, 30 s-320 h, 40 s-300 h, 1 min-280 h, 1 min-260 h, 1 min-240 h, 1 min-220 h, 1 min-200 h, 1 min-180 h, 1 min-160 h, 1 min-140 h, 1 min-120 h, 2 min-260 h, 3 min-240 h, 4 min-220 h, 5 min-200 h, 10 min-180 h, 30 min-160h, 1-150 h, 1 h-140 h, 5 h-120 h, or 10 h-100 h. Preferably, the reaction time is 1-150h. More preferably, the reaction time is 8-120h, for example, 8, 10, 12, 14, 16, 18, 20, 22, 24, 26, 28, 30, 32, 34, 36, 38, 40, 42, 44, 46, 48, 50, 60, 70, 72, 80, 90, 96, 100, 110, or 120h. Still further preferably, the reaction time is 8-12h.

In some embodiments, in reaction formula III, the reaction time is 10 s-360 h; for example, 10 s-360 h, 20 s-340 h, 30 s-320 h, 40 s-300 h, 1 min-280 h, 1 min-260 h, 1 min-240 h, 1 min-220 h, 1 min-200 h, 1 min-180 h, 1 min-160 h, 1 min-140 h, 1 min-120 h, 2 min-260 h, 3 min-240 h, 4 min-220 h, 5 min-200 h, 10 min-180 h, 30 min-160 h, 1-150 h, 1h-140 h, 5h-120 h, or 10h-100 h. Preferably, the reaction time is 1-150 h. More preferably, the reaction time is 8-120 h, for example, 8, 10, 12, 14, 16, 18, 20, 22, 24, 26, 28, 30, 32, 34, 36, 38, 40, 42, 44, 46, 48, 50, 60, 70, 72, 80, 90, 96, 100, 110, or 120h. Still further preferably, the reaction time is 12-48 h.

In some embodiments, in reaction formula II, the reaction time is 5 s-400 h; for example, 10 s-380 h, 20s-360 h, 40 s-320 h, 1 min-300 h, 1 min-280 h, 1 min-260 h, 1 min-240 h, 1 min-220 h, 1 min-200 h, 1 min-180 h, 1 min-160 h, 1 min-140 h, 1 min-120 h, 1 min-100 h, 1 min-80 h, 1 min-60 h, 1 min-40 h, 5 min-40 h, 11 min-40 h, 16 min-40 h, 1 min-40 h, 1 min-40 h, 1 min-40 h, 1 min-40 h, 1 min-40 h, 5 min-100 h, 2 min-260 h, 3 min-240 h, 4 min-220 h, 5 min-200 h, 10 min-180 h, 30 min-160 h, 1h-150h, 1h-120 h, 1h-100 h, 1h-80 h, 1h-60 h, 1h-40 h, 1h-20 h, 1h-10 h, 1h-5 h, 5h-100 h, 5h-80 h, 5h-60 h, 5h-40h, 5h-20h, 5h-10h, 10h-100h, 10h-80h, 10h-60h, 10h-40h, 10h-20h, 20h-60h, 20h-40h, 10h-30h, or 10h-20h. Preferably, the reaction time is 10 min-20h, for example, 10 min, 11 min, 12 min, 13 min, 14 min, 15 min, 16 min, 17 min, 18 min, 19 min, 20 min, 30 min, 1 h, 2h, 3 h, 4h, 5 h, 6h, 7 h, 8h, 9 h, 10h, 11 h, 12h, 13 h, 14h, 15 h, 16h, 17 h, 18h, 19h, or 20h. In some preferred embodiments, the reaction time is 11 min; in other preferred embodiments, the reaction time is 16 min; in other preferred embodiments, the reaction time is 0.5h; in other preferred embodiments, the reaction time is 2h; in other preferred embodiments, the reaction time is 2.5h; in other preferred embodiments, the reaction time is 3h; in some other preferred embodiments, the reaction time is 4h; in some other preferred embodiments, the reaction time is 6-8h; in some other preferred embodiments, the reaction time is 7h; in some other preferred embodiments, the reaction time is 10h; in some other preferred embodiments, the reaction time is 12h.

In the method described in the present disclosure, when R₁ and R₂ are both ethyl, the compound of formula 1 is HL. The reaction shown in the following reaction formula III is conducted under the catalysis of ^{t}Bu-P₄ and the initiation of BnOH.

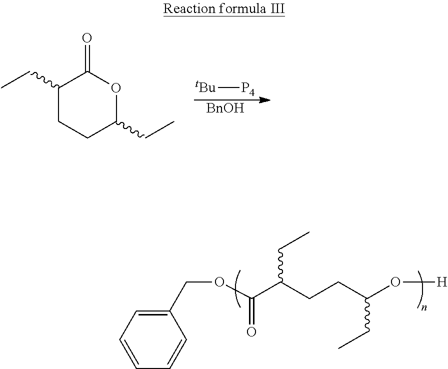

In the method described in the present disclosure, when R₁ and R₂ are both ethyl, the compound of formula 1 is HL. When R¹OH is used as the initiator, a monohydroxy end-capped polyester polymer compound is produced; and when HO—R²—OH is used as the initiator, a bis(hydroxy) end-capped polyester polymer compound is produced. The reaction is as follows:

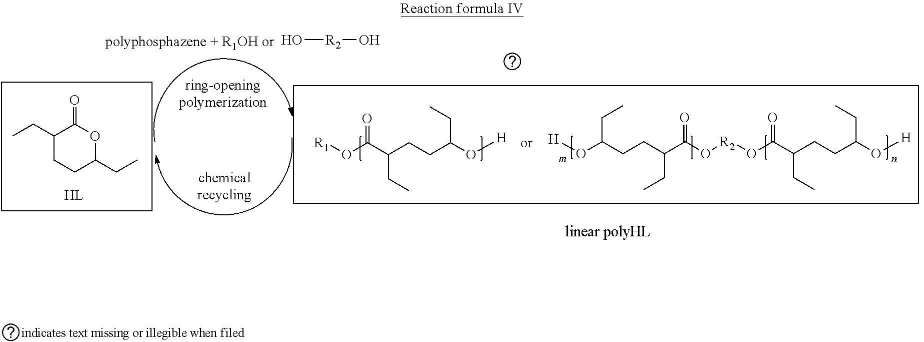

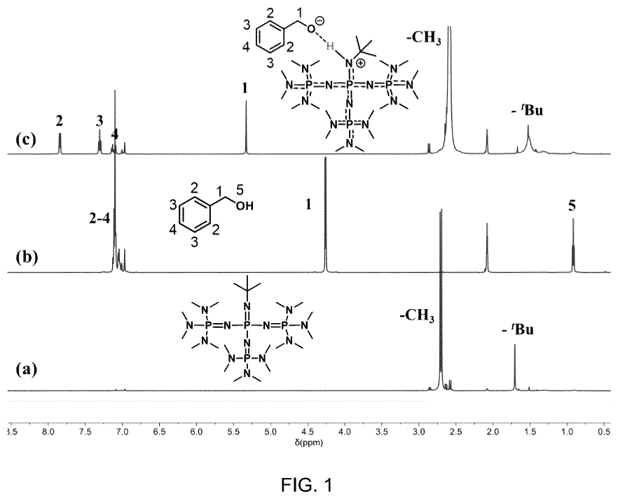

The possible chain initiation mechanism in the ring-opening polymerization of HL catalyzed by ^{t}Bu-P₄/BnOH of the present disclosure is shown in FIG. 1. The stoichiometric reaction of ^{t}Bu-P₄ with BnOH at room temperature is monitored by 1H NMR, and a complex [^{t}Bu-P₄H+ . . . . OBn] is formed. The disappearance of δ 0.92 ppm hydroxyl H as well as other signaling chemical shifts of ^{t}Bu-P₄ and BnOH are also detected.

The equivalent experimental procedure used to corroborate the mechanism includes the following steps: 0.05 mmol of BnOH and 0.05 mmol ^{t}Bu-P₄ are mixed, dissolved in 0.6 mL of toluene-d8, loaded into an NMR tube, and fully shaken for NMR testing, which is shown in FIG. 1.

In the method described in the present disclosure, when R₁₄ (OH)ₘ and/or (MO)ₘR₁₄ is used as the initiator and m is 1, monohydroxy end-capped CO₂-based polyester poly(δLH₂) is produced; when R₁₄ (OH)ₘ and/or (MO)ₘR₁₄ is used as the initiator and m is an integer greater than 1, polyhydroxy end-capped CO₂-based polyester poly(δLH₂) is produced; and the reaction is shown in Formula III.

In the method described in the present disclosure, when R₁ and R₂ are both ethyl, the compound of formula 1 is HL. The reaction shown in the following reaction formula V is conducted under the catalysis of ^{t}Bu-P₄.

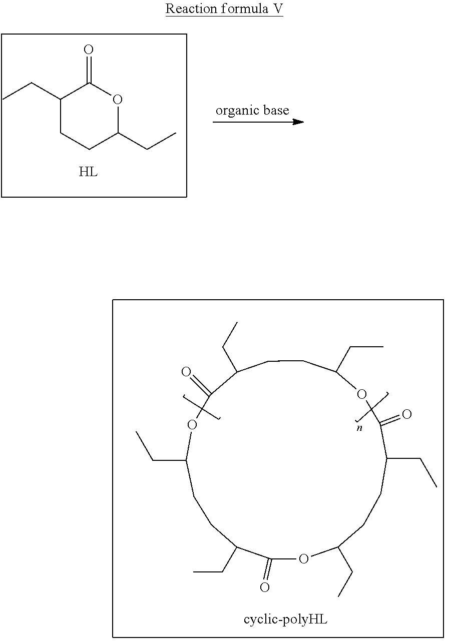

The definition of n is as described above.

The possible chain initiation mechanism in the ring-opening polymerization of HL catalyzed by [^{t}Bu-P₄H]⁺ of the present disclosure is shown in FIGS. 22-24. HL and ^{t}Bu-P₄ are mixed in different molar ratios at room temperature, respectively, the reaction is monitored by 1H and ³¹P NMR spectra, and the characteristic signals of [^{t}Bu-P₄H]⁺ and Bu-P₄ are monitored. The results indicate that ^{t}Bu-P₄ deprotonates a very small fraction of the HL monomers.

The present disclosure also provides the cyclic polyester polymer compound prepared by the method described above. The method allows to regulate the degree of polymerization of the cyclic polymer, thereby controlling the Mₙ (number average molecular weight) and Ð (molecular weight distribution) of the cyclic polymer. In some embodiments, Mₙ of the cyclic polymer is 30-2000 kg mol⁻¹. In some embodiments, Mₙ of the cyclic polymer is 30-100 kg mol⁻¹. In some embodiments, Mₙ of the cyclic polymer is 100-200 kg mol⁻¹. In some embodiments, Mₙ of the cyclic polymer is 200-400 kg mol⁻¹. In some other preferred embodiments, Mₙ of the cyclic polymer is 500-600 kg mol⁻¹. In some other preferred embodiments, Mₙ of the cyclic polymer is 500-800 kg mol⁻¹. In some other preferred embodiments, Mₙ of the cyclic polymer is 400-1500 kg mol⁻¹. In some other preferred embodiments, Mₙ of the cyclic polymer is 300-2000 kg mol⁻¹.

The present disclosure also provides a use of the cyclic polyester polymer compound of formula 3 or the cyclic polyester polymer compound prepared by the method described above in the preparation of any one or more of a polymer film, a pressure-sensitive adhesive (such as an adhesive tape), and a thermoplastic elastomer; where the pressure-sensitive adhesive can be further used in the preparation of products such as adhesive tapes. In some embodiments, the polymer film is colorless and transparent, as well as has good flexibility and viscoelasticity. In some embodiments, the adhesive tape is colorless and transparent, as well as has good flexibility and viscoelasticity.

The present disclosure also provides a polyester polymer compound prepared by the method described above.

The present disclosure also provides a CO₂-based polyester poly(δLH₂) prepared by the method described above.

The present disclosure also provides a use of the polyester polymer compound or the polyester polymer compound prepared by the method in the preparation of one or more of polyurethanes, polymer films, pressure-sensitive adhesives, adhesive tapes, and thermoplastic elastomers.

The poly(δLH₂) described in the present disclosure is a flexible material having a variety of excellent physicochemical properties, including high transparency, high molecular weight, good thermal stability, and good ductility. The present disclosure correspondingly provides a use of the CO₂-based polyester poly(δLH₂) or CO₂-based polyester poly(δLH₂) prepared by the above method in the field of polyurethanes and pressure sensitive adhesives.

Polyurethane field: polyurethane is a kind of polymer compound, mainly comprising polyester type and polyether type. Polyurethane is usually obtained through the copolymerization reaction between polyester polyol or polyether polyol and multifunctional isocyanate (such as diphenylmethane diisocyanate MDI, toluene diisocyanate TDI, etc.), therefore, polyester polyol or polyether polyol with different molecular weights is the indispensable raw material in the field of polyurethane synthesis.

According to the material properties of polyurethane, it includes rigid foam, soft foam, polyurethane elastomers, thermoplastics, polyurethane adhesives, polyurethane coatings, and paints. Polyurethane finds wide application in various fields such as automobile manufacturing, furniture, construction, thermal insulation, footwear manufacturing, etc., making it highly valuable in various applications.

Polyester polyols and polyether polyols are important precursors used in industry to synthesize polyurethane materials, and polyurethane materials prepared from polyester polyols often have better mechanical properties than polyurethane materials prepared from polyether polyols. However, the cost of polyester polyols is much higher than that of polyether polyols, thus limiting the large-scale application of polyester polyols. The polyHL in the present disclosure is cheap, easy to obtain, and has a new chemical structure, being of great and far-reaching significance to the field of polyurethane as well as to the field of the chemical industry.

Pressure-sensitive adhesive, commonly known as non-drying adhesive, is an indispensable functional material in the polymer industry. In terms of chemical structure, the pressure-sensitive adhesives currently on the market are predominantly based on polyolefin, which cannot be degraded after use. However, based on the preliminary experimental results of the present disclosure, it has been confirmed that polyHL exhibits relatively good properties of pressure sensitive adhesive and can be chemically recycled into its monomers. Therefore, it has the potential to become the first type of pressure-sensitive adhesive on the market capable of monomer recycling, with promising prospects for industrialization.

The adhesive tape may be a transparent adhesive tape, an opaque adhesive tape, a single-sided adhesive tape, a double-sided adhesive tape, a protective adhesive tape, a heat-insulating adhesive tape, a high-temperature adhesive tape, a masking adhesive tape, an electrical adhesive tape, an electroplating adhesive tape, a packaging adhesive tape, a cloth-based adhesive tape, a fiber-based adhesive tape, a PE foam adhesive tape, a kraft adhesive tape, a protective film adhesive tape, a special adhesive tape, and the like. In some other preferred embodiments, the tape is a transparent adhesive tape. In yet other preferred embodiments, the tape is a 3M commercially available transparent tape. The polymer film is colorless and transparent and has good flexibility and viscoelasticity (peel strength).

The present disclosure also provides a use of organic bases such as polyphosphazene in catalyzing a reaction for synthesizing polyester polymer compounds of formula 2 and/or formula 3 from the compound of formula 1. The organic base may be a sterically hindered base or a non-nucleophilic base.

The organic base is an amine compound or a nitrogen-containing heterocyclic compound; where the amine compound has a structure of the following formula:

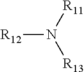

Ammonium salt has a structure of R₁₁R₁₂R₁₃N⁺H, where R₁, R₁₂, and R₁₃ each independently is a hydrogen (H), C1-C20 alkyl, C5-C20 cycloalkyl, or C7-C20 alkylaryl. These groups each optionally comprises one or more heteroatoms (e.g., oxygen, phosphorus, or sulphur atom) and/or substituents. A ring may exist between R₁₁ and R₁₂, R₁₂ and R₁₃, and/or R₁₁ and R₁₃, where the ring may comprise a heteroatom.

Preferably, the organic base is selected from one or more of a phosphazene, a compound containing a guanidine group, and a compound containing an amidino group. In some embodiments, the organic base is selected from ^{t}Bu-P4, 1,5,7-triazabicyclo[4.4.0]deca-5-ene (TBD), 1,8-diazabicycloundec-7-ene (DBU), diethylamine, dimethylamine, triethylamine, N,N-diisopropylethylamine, N-methyl morpholine, n-octylamine, tri-n-butylamine, laurylamine, stearylamine, tetra-propylammonium hydroxide (TPAOH), tetra-butylammonium hydroxide (TBAOH), sodium alcoholate or potassium alcoholate of C1˜C5, triethanolamine, choline, N-methylmorpholine, pyridine, dimethylaminopyridine, N, N′-dihydroxyethyl ethylenediamine, β-hydroxyethyl ethylenediamine, N-(2-hydroxyethyl)ethylenediamine, N, N,N′,N′-tetrahydroxyethyl ethylenediamine, N-hydroxyethyl propylenediamine, trimethylhydroxyethyl propylenediamine, and N,N′-bis-(2-hydroxyethyl)-1,3-propylenediamine.

Preferably, the organic base is one or more of phosphazene and TBD. Further preferably, the phosphazene is a strong Lewis base having the structure of (R₂N)₂₋P═N. Further preferably, the phosphazene has a structure shown as formula 5:

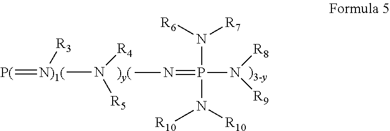

In formula 5, R₃—R₁₀ are independently selected from alkyl, preferably C1-C10 alkyl, such as C1 (methyl), C2 (ethyl), C3 (propyl, isopropyl), C4 (butyl, tert-butyl), C5, C6, C7, C8, C9, C10 alkyl, respectively.

y is a positive integer no less than 1. For example, 1≤y≤10, 10≤y≤20. Preferably, 1≤y≤3.

Preferably, the phosphazene is selected from ^{t}Bu-P₁, ^{t}Bu-P₂, ^{t}Bu-P₄, with the structures shown below, respectively:

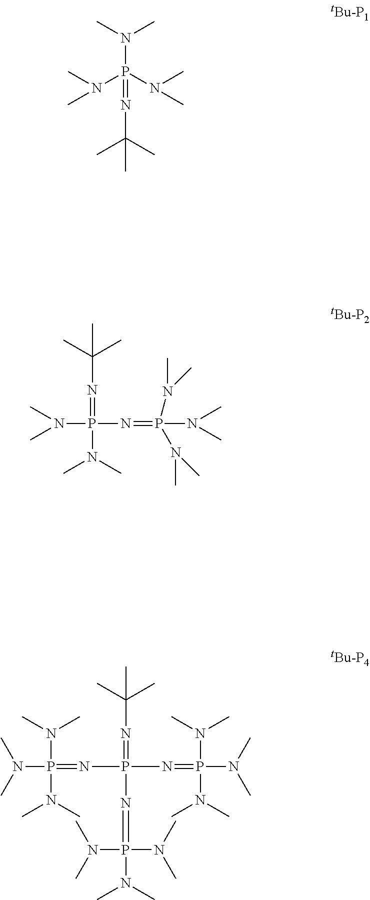

The present disclosure also provides methods for catalyzing the polymer to recycle the monomer, the method is selected from one or more of the following:
a) the method for catalyzing the polymer of formula 2 comprises a catalytic method and a pyrolysis method (thermolysis);
b) the method for catalyzing the polymer of formula 3 comprises an inorganic salt pyrolysis catalytic method and a La[N(SiMe₃)₂]₃ mild catalytic method.

In the present disclosure, the catalytic method in a) comprises: degrading the polyester polymer compound of formula 2 by using an inorganic salt, a metal organic compound, or an organic compound catalyst, to obtain the heterocyclic monomers of formula 1, or an oligomer or a derivative thereof.

Specifically, in a), when the polyester polymer compound is CO₂-based polyester poly(δLH₂), the δLH₂ monomers are recycled from polymer Poly(δLH₂), and the reaction is shown in reaction formula VI below:

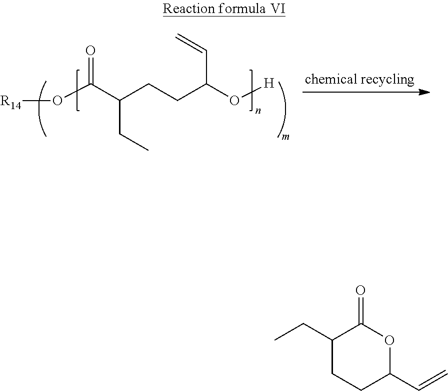

The catalytic method comprises: degrading the CO₂-based polyester poly(δLH₂) of formula 8 by employing at least one of an inorganic salt, a metal organic compound, or an organic compound catalyst to obtain δLH₂ of formula 7, or oligomers or derivatives thereof.

In a), the pyrolysis method comprises: pyrolyzing the polyester polymer compound of formula 2 to obtain the heterocyclic monomers of formula 1, or an oligomer or a derivative thereof. The catalytic method comprises: degrading the polyester polymer compound of formula 2 in a solvent by employing a catalyst such as an inorganic salt, a metal organic compound, an organic compound, etc. to obtain a heterocyclic compound of formula 1, or an oligomer or a derivative thereof.

Specifically, in a), when the polyester polymer compound is a CO₂-based polyester poly(δLH₂), the pyrolysis method comprises: pyrolyzing the CO₂-based polyester poly(δLH₂) of formula 8 to obtain δLH₂ of formula 7, or oligomers or derivatives thereof.

In a), the solvent is selected from one or more of benzene, chlorobenzene, bromobenzene, dichlorobenzene, dibromobenzene, ortho-dichlorobenzene, ortho-dibromobenzene, meta-dichlorobenzene, meta-dibromobenzene, para-dichlorobenzene, para-dibromobenzene, toluene, meta-dimethyl benzene, para-dimethyl benzene, ortho-dimethyl benzene, mesitylene, dimethylsulfoxide, N,N-dimethylformamide, N,N-dimethylacetamide, and the like; preferably, toluene or ortho-dichlorobenzene; more preferably, ortho-dichlorobenzene. When the polyester polymer compound is a CO₂-based polyester poly(δLH₂), the solvent is more preferably toluene.

In a), the salt is an inorganic salt, and the inorganic salt is selected from one or more of zinc chloride, tin chloride, copper chloride, nickel chloride, cuprous chloride, palladium chloride, platinum chloride, yttrium chloride, ferric chloride, ferrous chloride, titanium trichloride, zirconium chloride, lanthanum trichloride, lanthanum aluminum oxide, lanthanum fluoride, lanthanum borate, lanthanum sulfate, lanthanum hydroxide, lanthanum carbonate, lanthanum oxalate, lanthanum acetate, lanthanum bromide, lanthanum nitrate, and the like; preferably, zinc chloride.

In a), the metal organic compound is selected from one or more of stannous octanoate, stannous isooctanoate, dibutyltin dilaurate, bis[bis(trimethylmethylsilyl)amino]tin, lanthanum triisopropoxide, lanthanum tris[N,N-bis(trimethylsilane)amine], lanthanum trifluoromethanesulfonate, silver trifluoromethanesulfonate, copper trifluoromethanesulfonate, iron trifluoromethanesulfonate, scandium trifluoromethanesulfonate, yttrium trifluoromethanesulfonate and the like; preferably stannous octanoate or tris[N,N-bis(trimethylsilane)amine]lanthanum.

In a), the organic compound is selected from one or more of 1,8-diazabicyclo[5.4.0]undec-7-ene (DBU), 1,5,7-triazabicyclo(4.4.0) dec-5-ene (TBD), 1,3-dimesitylimidazol-2-ylidene (IMes), ^{t}Bu-P₁, ^{t}Bu-P₂, ^{t}Bu-P₄, etc.; preferably, DBU.

When the polyester polymer compound is a CO₂-based polyester poly(δLH₂), the catalyst is an inorganic salt; preferably, the catalyst is a lanthanum salt; further preferably, the catalyst is La[N(SiMe₃)₂]₃.

In a), the degradation is performed in a hot bath, such as an oil bath or a sand bath, with a temperature ranging from 100° C. to 300° C., preferably, 120° C. to 300° C., more preferably, 160° C. When the polyester polymer compound is a CO₂-based polyester poly(δLH₂), the temperature is in a range of 120-180° C., preferably, 120° C.

In a), the degradation time is in a range of 8-24 h; preferably, 12h. When the polyester polymer compound is a CO₂-based polyester poly(δLH₂), the degradation time is in a range of 0.5-24 h; preferably, 0.5-8 h; more preferably, 1-5 h; further preferably, 2 h.

In the present disclosure, in a), the pyrolysis method comprises: pyrolyzing the polyester polymer compound to obtain the heterocyclic monomers of formula 1, an oligomer thereof, or a derivative corresponding to formula 1.

Preferably, the pyrolyzing is carried out in a nitrogen atmosphere.

Preferably, the pyrolyzing is carried out under sealing conditions.

Preferably, the pyrolyzing is carried out under vacuum conditions.

The pyrolyzing is preferably carried out in a sand bath. In the present disclosure, there is no particular limitation on the heating temperature, as long as the monomers of the polyester polymer compound can be recycled. In some embodiments, the heating temperature is greater than 100° C. In other embodiments, the heating temperature is in a range of 100-1000° C.; for example, 100-200, 200-300, 150-300, 300-400, 400-500, 500-600, 600-700, 700-800, 800-900, or 900-1000° C. Preferably, the heating temperature is 150-300° C. Further preferably, the heating temperature is 150-260° C. Further preferably, the heating temperature is 180-230° C. Further preferably, the heating temperature is 200-230° C. Further preferably, the heating temperature is 220° C.

In the present disclosure, there is no particular limitation on the heating duration, as long as the polyester polymer compound can be pyrolyzed to recycle the corresponding monomers. In some embodiments, the heating time is greater than 0.5h. In other embodiments, the heating time is in a range of 1-50h; for example, 1-5, 5-10, 10-15, 15-20, 1-20, 20-25, 25-30, 30-35, 35-40, 40-45, or 45-50 h. Preferably, the heating time is 1-20 h. Further preferably, the heating time is 1-10 h. Further preferably, the heating time is 1-5 h. Further preferably, the heating time is 3 h.

In a preferred embodiment, catalyzing the CO₂-based polyester poly(δLH₂) to recycle the monomer δLH₂ by using the catalytic method comprises: cracking the CO₂-based polyester poly(δLH₂) of formula 8 in toluene for 2 h at 120° C. under the catalysis of La[N(SiMe₃)₂]₃ to obtain the δLH₂ of formula 7, an oligomer thereof, or an derivative corresponding to formula 7.

In b), the inorganic salt pyrolysis catalytic method comprises: degrading the polyester polymer compound of formula 3 under the catalysis of an inorganic salt to obtain a heterocyclic compound of formula 1, an oligomer thereof, or a derivative corresponding to formula 1.

In b), the La[N(SiMe₃)₂]₃ mild catalytic method comprises: degrading the polyester polymer compound of formula 3 under the catalysis of La[N(SiMe₃)₂]₃ under mild conditions to obtain the heterocyclic compound of formula 1, the oligomer thereof, or the derivative corresponding to formula 1.

The pyrolysis catalytic method can be carried out in solvent or solvent-free conditions.

In b), when the degradation is carried out in a solvent, the solvent may be selected from one or more of toluene, benzene, chlorobenzene, bromobenzene, dichlorobenzene, dibromobenzene, ortho-dichlorobenzene, ortho-dibromobenzene, meta-dichlorobenzene, meta-dibromobenzene, para-dichlorobenzene, para-dibromobenzene, toluene, meta-dimethyl benzene, para-dimethyl benzene, ortho-dimethyl benzene, mesitylene, dimethylsulfoxide, N,N-dimethylformamide, N,N-dimethylacetamide, and the like. In some preferred embodiments, the solvent is toluene. In some other preferred embodiments, the solvent is mesitylene. In some other preferred embodiments, the solvent is ortho-dichlorobenzene.

In the present disclosure, there is no particular limitation on the initial concentration [M]₀ of the cyclic polymer of formula (3) when using the pyrolysis catalytic method, as long as the preparation of the cyclic polymer can be achieved. In some embodiments, the initial concentration [M]₀ of the cyclic polymer of formula (3) is in a range of 1-50 mol L⁻¹, for example, 2, 4, 6, 8, 10, 12, 14, 16, 18, 20, 22, 24, 26, 28, 30, 32, 34, 36, 38, 40, 42, 44, 46, 48, or 50 mol L⁻¹. Preferably, the initial concentration [M]₀ of the cyclic polymer of formula (2) is 2-40 mol L⁻¹; more preferably, 3-30 mol L⁻¹; further preferably, 4-20 mol L⁻¹. In some preferred embodiments, the initial concentration [M]₀ of the cyclic polymer is 3-6 mol L⁻¹. In yet other preferred embodiments, the initial concentration [M]₀ of the heterocyclic lactone is 4.0 mol L⁻¹.

The pyrolysis and catalysis are performed in a hot bath, such as an oil bath or a sand bath, with a temperature ranging from 100° C. to 350° C. Preferably, the temperature is in a range of 110-300° C.; more preferably, 120-240° C.; for example, 120, 30, 140, 150, 160, 170, 180, 190, 200, 210, 220, 230, or 240° C. In some preferred embodiments, the pyrolysis and catalysis temperature is 120° C. In some preferred embodiments, the pyrolysis and catalysis temperature is 130° C. In some preferred embodiments, the pyrolysis and catalysis temperature is 140° C. In some preferred embodiments, the pyrolysis and catalysis temperature is 150° C. In some preferred embodiments, the pyrolysis and catalysis temperature is 160° C. In some preferred embodiments, the pyrolysis and catalysis temperature is 180° C.

The pyrolysis and catalysis time is in a range of 6-45h; preferably, 8-36h, for example, 8, 9, 10, 11, 12, 13, 14, 15, 16, 17, 18, 19, 20, 21, 22, 23, 24, 25, 26, 27, 28, 29, 30, 31, 32, 33, 34, 35, or 36h. In some preferred embodiments, the pyrolysis and catalysis time is 10h. In some preferred embodiments, the pyrolysis and catalysis time is 12h. In some preferred embodiments, the pyrolysis and catalysis time is 16h. In some preferred embodiments, the pyrolysis and catalysis time is 24h.

In the present disclosure, there is no particular limitation on the physicochemical properties of the cyclic polymer when using the inorganic salt pyrolysis catalytic method, as long as monomers can be recycled from cyclic polymers obtained in accordance with the method described above or other methods of the present disclosure. In some preferred embodiments, the inorganic salt pyrolysis catalytic method of the present disclosure is applicable to cyclic polymers with Mₙ in the range of 300-2000 kg mol⁻¹. In some preferred embodiments, the inorganic salt pyrolysis catalytic method of the present disclosure is applicable to cyclic polymers with Mₙ in the range of 400-1500 kg mol⁻¹. In some preferred embodiments, the inorganic salt pyrolysis catalytic method of the present disclosure is applicable to cyclic polymers with Mₙ in the range of 500-800 kg mol⁻¹. In some preferred embodiments, the inorganic salt pyrolysis catalytic method of the present disclosure is applicable to cyclic polymers with Mₙ in the range of 500-600 kg mol⁻¹. In some preferred embodiments, the inorganic salt pyrolysis catalytic method of the present disclosure is applicable to cyclic polymers prepared in Embodiments 1 to 17 of the present disclosure.

Optionally, the pyrolysis and catalysis are carried out in a nitrogen atmosphere.

Optionally, the pyrolysis and catalysis are carried out under sealing conditions.

Optionally, the pyrolysis and catalysis are carried out under vacuum conditions.

In the present disclosure, the La[N(SiMe₃)₂]₃ mild catalytic method comprises: degrading the cyclic polymer as described above under the catalysis of La[N(SiMe₃)₂]₃ under mild conditions to obtain a heterocyclic compound of formula 1, oligomer thereof, or a derivative corresponding to formula 1.

The La[N(SiMe₃)₂]₃ mild catalytic method can be carried out in solvent or solvent-free conditions.

When the degradation is carried out in a solvent, the solvent may be selected from one or more of toluene, benzene, chlorobenzene, bromobenzene, dichlorobenzene, dibromobenzene, ortho-dichlorobenzene, ortho-dibromobenzene, meta-dichlorobenzene, meta-dibromobenzene, para-dichlorobenzene, para-dibromobenzene, toluene, meta-dimethyl benzene, para-dimethyl benzene, ortho-dimethyl benzene, mesitylene, dimethylsulfoxide, N,N-dimethylformamide, N,N-dimethylacetamide, and the like. In some preferred embodiments, the solvent is toluene. In some other preferred embodiments, the solvent is mesitylene.

In the present disclosure, there is no particular limitation on the initial concentration [M]₀ of the cyclic polymer of formula (3) when using the mild catalytic method, as long as the preparation of the cyclic polymer can be achieved. In some embodiments, the initial concentration [M]₀ of the cyclic polymer of formula (2) is in a range of 1-50 mol L⁻¹, for example, 2, 4, 6, 8, 10, 12, 14, 16, 18, 20, 22, 24, 26, 28, 30, 32, 34, 36, 38, 40, 42, 44, 46, 48, or 50 mol L⁻¹. Preferably, the initial concentration [M]₀ of the cyclic polymer of formula (2) is 2-40 mol L⁻¹; more preferably, 3-30 mol L⁻¹; further preferably, 4-20 mol L⁻¹. In some preferred embodiments, the initial concentration [M]₀ of the cyclic polymer is 3-6 mol L⁻¹. In yet other preferred embodiments, the initial concentration [M]₀ of the heterocyclic lactone is 4.0 mol L⁻¹.

The mild catalysis is performed in a hot bath, such as an oil bath or a sand bath, with a temperature ranging from 40° C. to 90° C., preferably, 50-80° C., for example, 50, 55, 60, 65, 70, 75, or 80° C. In some preferred embodiments, the degradation temperature is 50° C. In some preferred embodiments, the degradation temperature is 60° C. In some preferred embodiments, the degradation temperature is 70° C. In some preferred embodiments, the degradation temperature is 80° C.

The mild catalysis time is in a range of 1-45h, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45h. In some preferred embodiments, the degradation time is 3h. In some preferred embodiments, the degradation time is 6h. In some preferred embodiments, the degradation time is 9h. In some preferred embodiments, the degradation time is 12h. In some preferred embodiments, the degradation time is 15h. In some preferred embodiments, the degradation time is 18h. In some preferred embodiments, the degradation time is 21h. In some preferred embodiments, the degradation time is 24h.

In the present disclosure, there is no particular limitation on the physicochemical properties of the cyclic polymer when using the mild catalytic method, as long as monomers can be recycled from cyclic polymers obtained in accordance with the method described above or other methods of the present disclosure. In some preferred embodiments, the mild catalytic method of the present disclosure is applicable to cyclic polymers with Mₙ in the range of 300-2000 kg mol⁻¹. In some preferred embodiments, the mild catalytic method of the present disclosure is applicable to cyclic polymers with Mₙ in the range of 400-1500 kg mol⁻¹. In some preferred embodiments, the mild catalytic method of the present disclosure is applicable to cyclic polymers with Mₙ in the range of 500-800 kg mol⁻¹. In some preferred embodiments, the mild catalytic method of the present disclosure is applicable to cyclic polymers with Mₙ in the range of 500-600 kg mol⁻¹. In some preferred embodiments, the mild catalytic method of the present disclosure is applicable to cyclic polymers prepared in Embodiments 1 to 17 of the present disclosure. Optionally, the mild catalysis is carried out in a nitrogen atmosphere.

Optionally, the mild catalysis is carried out under sealing conditions.

Optionally, the mild catalysis is carried out under vacuum conditions.

In the method described in the present disclosure, when catalyzing the polymer PolyHL to recycle Poly monomer, the reaction is selected from the following reaction formula VII and reaction formula VIII:

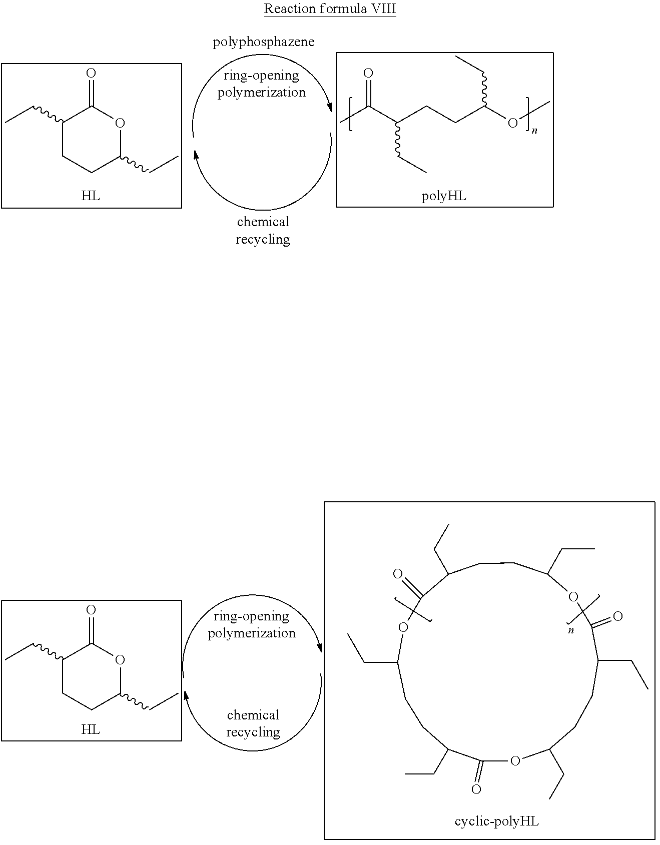

When the polyester polymer compound is the CO₂-based polyester poly(δLH₂), there is no specific limitation on the δ-L feedstock used in the following embodiments, for example, δ-L can be obtained commercially or prepared from a reaction between CO₂ and 1,4-butadiene under the catalysis of Pd. δLH₂ is then obtained with Stryker reagent as catalyst and triethoxysilane (HSi(OEt)₃) as the hydrogen source. The reaction is shown in the following reaction pathway 4.

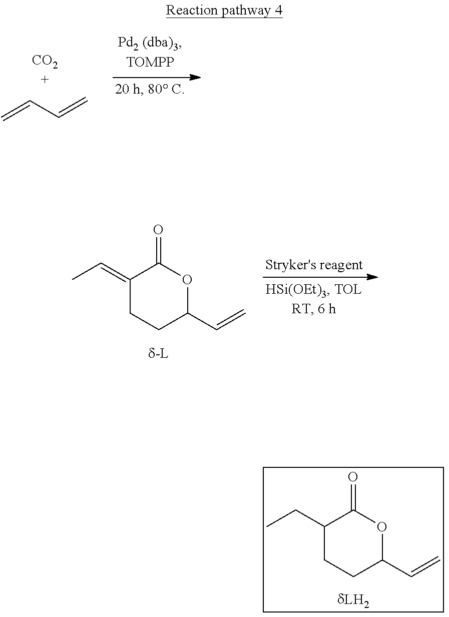

When the polyester polymer compound is the CO₂-based polyester poly(δLH₂), the present disclosure provides a disubstituted α,β-saturated six-membered cyclic lactone having the structure of formula 10 below:

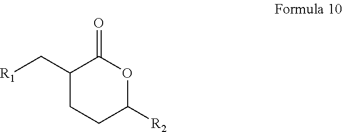

R₁, R₂ are each independently selected from any one of hydrogen, halogen, alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted and polyheterocyclic aromatic group. The monocyclic aromatic group includes phenyl, aza-aromatic group, thio-aromatic group, and oxa-aromatic group, and the polycyclic aromatic group and polyheterocyclic aromatic group refer to groups comprising two or more monocyclic aromatic groups.

The halogen is selected from fluorine, chlorine, bromine, and iodine.

The alkyl may be linear, branched, or cyclic; further, the alkyl may be C1 to C20 alkyl or C1 to C10 alkyl. Examples include methyl, ethyl, propyl, butyl, pentyl, hexyl, heptyl, octyl, nonyl, and decyl, with ethyl being preferred.

The substituted alkyl may be substituted linear alkyl, substituted branched alkyl, or substituted cyclic alkyl.

The alkenyl may be linear, branched, cyclic; further, the alkenyl may be C1 to C20 or C1 to C10 alkenyl. Examples include ethenyl, propenyl, butenyl, pentenyl, hexenyl, heptenyl, octenyl, nonenyl, and decenyl; with ethenyl being preferred.

The substituted alkenyl may be substituted linear alkenyl, substituted branched alkenyl, or substituted cyclic alkenyl.

The alkynyl may be linear, branched, cyclic; further, the alkynyl may be C1 to C20 or C1 to C10 alkynyl. Examples include ethynyl, propynyl, butynyl, pentynyl, hexynyl, heptynyl, octynyl, nonynyl, and decynyl; with ethynyl being preferred.

The substituted alkynyl group may be substituted linear alkynyl, substituted branched alkynyl, or substituted cyclic alkynyl.

The alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfonylamino group, sulfonamide group, morpholinyl, and piperazinyl. Further, the alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, hydroxyl, nitro, halo, carboxyl, C1-C10 alkyl, alkoxyl, amine, cycloalkoxyl, cycloalkylamine group, C2-C12 alkenyl, C2-C12 alkynyl, C3-C12 cycloalkyl, benzyl, alkyl carbonyl, C2-C12 alkenyl carbonyl, C3-C12 cycloalkyl carbonyl, phenyl carbonyl, benzyl carbonyl, alkoxyl carbonyl, esteryl, sulfinyl, sulfonyl, sulfinamide group, sulfonamide group, morpholinyl, and piperazinyl.

Preferably, R₁ is C1-C20 alkyl, R₂ is C1-C20 alkenyl; further preferably, R₁ is C1-C10 alkyl, for example, C1, C2, C3, C4, C5, C6, C7, C8, C9, or C10 alkyl, and R₂ is C1-C10 alkenyl, for example, C1, C2, C3, C4, C5, C6, C7, C8, C9, or C10 alkenyl. Further preferably, R₁ is methyl and R₂ is ethenyl.

When the polyester polymer compound is the CO₂-based polyester poly(δLH₂), the present disclosure also provides a method for preparing the disubstituted α,β-saturated six-membered cyclic lactone of formula 10. The method uses disubstituted α,β-unsaturated six-membered cyclic lactone of formula 9 as reactant to perform a selective reduction reaction of conjugated olefins, so as to obtain the disubstituted α,β-saturated six-membered cyclic lactones of formula 10, where Stryker reagent or a mixture of reagents capable of in situ producing Stryker reagent serves as a catalyst, and organosilane serves as the hydrogen source. The reaction is shown as the reaction formula A.

R₁, R₂ are defined as described above in formula 10.

The disubstituted α,β-saturated six-membered cyclic lactone is a six-membered cyclic lactone with double-group substitution.

In some embodiments, the disubstituted α,β-saturated six-membered cyclic lactone is a mixture of diastereoisomers.

The Stryker reagent has the chemical formula of [(Ph₃P)CuH]₆.

The mixture of reagents capable of producing the Stryker reagent in situ comprises CuXp and a phosphine ligand; where p=0, 1, or 2, X is any one of a halogen atom, an anionic species, or a ligand compound. Preferably, X is a halogen atom, such as F, Cl, Br, or I; an anionic species, such as sulfate, sulfite, bisulfite, nitrate, acetate, etc.; or a ligand compound, such Is acetylacetonate anion, bisdibenzylideneacetone, triphenylphosphine, triethylphosphine, triethoxyphosphine, BINAP, and the like.

The organosilane is a compound containing a silicon hydrogen bond; preferably, the organosilane is SiH_{q}(R₀)ₜ, where q and t are integers ranging from 0 to 5 and q+t=4, and where R₀ may represent halogen atoms such as fluorine, chlorine, bromine, and iodine, or other groups such as alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group. The monocyclic aromatic group includes phenyl, aza-aromatic group, thio-aromatic group, and oxa-aromatic group, and the polycyclic aromatic group and polyheterocyclic aromatic group refer to groups comprising two or more monocyclic aromatic groups.

The halogen is selected from fluorine, chlorine, bromine, and iodine.

The alkyl may be linear, branched, or cyclic; further, the alkyl may be C1 to C20 alkyl or C1 to C10 alkyl. Examples include methyl, ethyl, propyl, butyl, pentyl, hexyl, heptyl, octyl, nonyl, and decyl, with ethyl being preferred.

The substituted alkyl may be substituted linear alkyl, substituted branched alkyl, or substituted cyclic alkyl.

The alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide, sulfonamide group, morpholinyl, and piperazinyl. Further, the alkyl, alkenyl, alkynyl, monocyclic aromatic group, polycyclic aromatic group, and polyheterocyclic aromatic group are mono-substituted or poly-substituted, and the substituent is independently selected from one or more of the following: hydrogen, heteroatom, amino, cyano, hydroxyl, nitro, halo, carboxyl, C1-C10 alkyl, alkoxyl, amine, cycloalkoxyl, cycloalkylamine group, C2-C12 alkenyl, C2-C12 alkynyl, C3-C12 cycloalkyl, benzyl, alkyl carbonyl, C2-C12 alkenyl carbonyl, C3-C12 cycloalkyl carbonyl, phenyl carbonyl, benzyl carbonyl, alkoxyl carbonyl, esteryl, sulfinyl, sulfonyl, sulfinamide group, sulfonamide group, morpholinyl, and piperazinyl.

Preferably, R₀ is C1-C20 alkyl. Further preferably, R₀ is C1-C10 alkyl, for example, C1, C2, C3, C4, C5, C6, C7, C8, C9, or C10 alkyl.

In the method for preparing the disubstituted α,β-saturated six-membered cyclic lactone as described in the present disclosure, the Stryker reagent can be produced in situ from CuXp and a phosphine ligand; where p=0, 1, or 2, X is a halogen atom, such as F, Cl, Br, or I; an anionic species, such as sulfate, sulfite, bisulfite, nitrate, acetate, etc.; or a ligand compound, such as acetylacetonate anion, bisdibenzylideneacetone, triphenylphosphine, triethylphosphine, triethoxyphosphine, BINAP, and the like. The disubstituted α,β-saturated six-membered cyclic lactones of formula 10 are prepared as shown in reaction formula A-1 below:

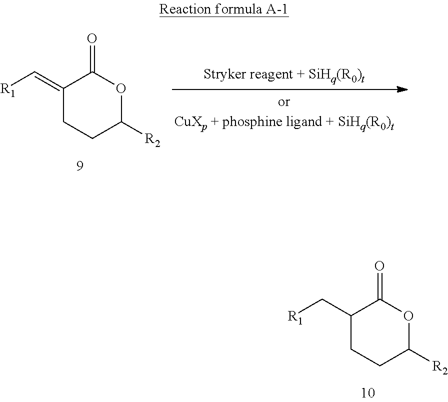

The mass ratio of the disubstituted α,β-unsaturated six-membered cyclic lactone, Stryker reagent, and organosilane is (0.5-30):(0.05-5):(3-40); preferably, (2-15):(0.06-1):(6-20), for example, (2-5):(0.06-1):(6-20), (5-8):(0.06-1):(6-20), (8-10):(0.06-1):(6-20), (10-15):(0.06-1):(6-20), (2-15):(0.08-0.1):(6-20), (2-15):(0.1-0.3):(6-20), (2-15):(0.3-0.5):(6-20), (2-15):(0.5-0.8):(6-20), (2-15):(0.8-1):(6-20), (2-15):(0.06-1):(6-8), (2-15):(0.06-1):(8-10), (2-15):(0.06-1):(10-12), (2-15):(0.06-1):(12-15), (2-15):(0.06-1):(15-20), further preferably, (3-10):(0.08-0.3):(8-15); such as 5:0.11:11.9.

There is no specific limitation on the temperature of the selective reduction reaction, as long as the preparation of the disubstituted α,β-saturated six-membered cyclic lactone can be achieved. In some embodiments, the temperature of the reaction is in a range of 0˜50° C., for example, 0˜10, 10˜20, 20˜30, 30˜40, or 40˜50° C. Preferably, the temperature of the reaction is 10˜40° C. Further preferably, the temperature of the reaction is 15˜35° C., for example, 15, 16, 17, 18, 19, 20, 21, 22, 23, 24, 25, 26, 27, 28, 29, 30, 31, 32, 33, 34, or 35° C. Further preferably, the temperature of the reaction is room temperature, such as 25±5° C. Further preferably, the temperature of the reaction is 25° C.

There is no specific limitation on the duration of the selective reduction reaction, as long as the preparation of the disubstituted α,β-saturated six-membered cyclic lactone can be achieved. In some embodiments, the time of the reaction is in a range of 30 s-160 h; for example, 30 s-10 min, 10 min-30 min, 30 min-1 h, 1 h-3 h, 3 h-6 h, 6 h-10 h, 10 h-30 h, 30 h-60 h, 60 h-90 h, 90 h-120 h, or 120 h-160 h. Preferably, the time of the reaction is 1 h-10 h. More preferably, the time of the reaction is 2-8 h. Further preferably, the time of the reaction is 6 h.

The solvent for the selective reduction reaction is selected from one or more of toluene, xylene, dichlorobenzene, mesitylene, dichloromethane, chloroform, tetrahydrofuran (THF), TBD, benzene, 1,2-dichloroethane, tetrahydropyrrole, tetrapyran, hexahydropyridine, ethyl acetate, ethyl ether, dimethyl ether, methyl ethyl ether, n-hexane, cyclohexane, cyclopentane, acetonitrile, dioxane, N,N-dimethylformamide, dimethyl sulphoxide, etc. Preferably, the solvent is toluene.

Preferably, the preparation process of the disubstituted α,β-saturated six-membered cyclic lactones in the present disclosure is carried out under stirring conditions.

Preferably, the process for preparing the disubstituted α,β-saturated six-membered cyclic lactone in the present disclosure specifically comprises, dissolving the Stryker reagent and the organosilane in a solvent, and then adding δ-L dropwise to the mixture to perform a reaction.

In a specific embodiment, when R₁ is methyl and R₂ is ethenyl in reaction formula A, the disubstituted α,β-saturated six-membered cyclic lactone of formula 6 is δLH₂, and the corresponding reaction is shown as reaction formula A-2 below:

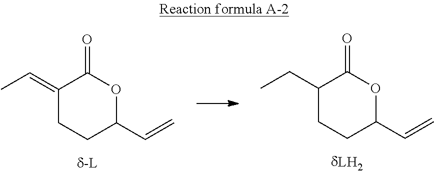

In another specific embodiment, when R₁ is methyl and R₂ is ethenyl in reaction formula A, the disubstituted α,β-saturated six-membered cyclic lactone of formula 6 is δLH₂, and the corresponding reaction is shown as reaction formula A-3 below:

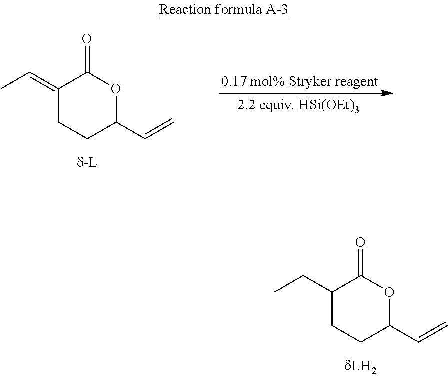

It should be understood that process equipment or devices not specifically noted in the following embodiments are conventional equipment or devices in the prior art. It also should be understood that the reference to one or more method steps in the present disclosure does not exclude the existence of other method steps before or after the combination steps mentioned, or the insertion of other method steps between these explicitly mentioned steps, unless otherwise specified. Before further describing specific embodiments of the present disclosure, it should be understood that the protection scope of the present disclosure is not limited to the particular specific embodiments described below. It should also be understood that the terminology used in the embodiments of the present disclosure is intended to be descriptive of particular embodiments and is not intended to limit the protection scope of the present disclosure. In the specification and the claims of the present disclosure, unless otherwise expressly stated in the text, the singular forms “a”, “one”, and “the” include the plural form.

When ranges of values are provided in the embodiments, it is to be understood that the two endpoints of each range of values, and any of the values between the two endpoints, may be selected unless otherwise indicated in the present disclosure. Unless otherwise defined, all technical and scientific terms used in the present disclosure have the same meaning as commonly understood by those skilled in the art. Except for the specific methods, devices, and materials used in the embodiments, according to the understanding of those skilled in the art and the content of the present disclosure, any methods, devices, and materials similar to or equivalent to those described in the embodiments of the present disclosure can also be used to implement the present disclosure.

There is no specific limitation on the HL feedstock used in the following embodiments, for example, HL can be obtained commercially or prepared from a reaction shown below. CO₂ reacts with 1,4-butadiene under the catalysis of Pd to produce 8-L, the produced δ-L then reacts with H₂ under the catalysis of Pd/C to obtain six-membered lactone 3,6-diethyl-tetrahydro-2h-pyran-2-one (HL). The reaction is shown in the following reaction pathway 5.

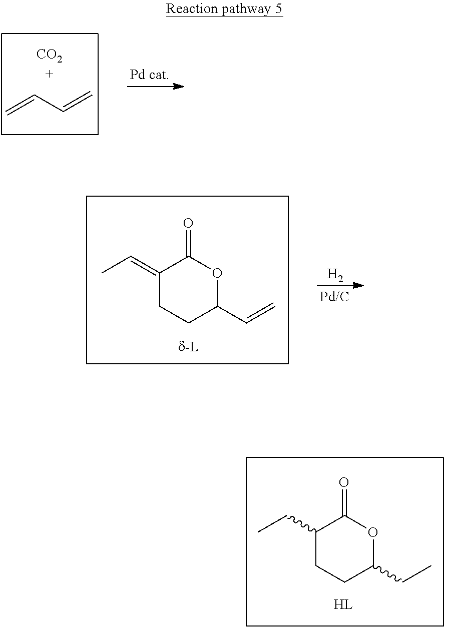

In the following embodiments, the compound polyHL is prepared by ring-opening polymerization of HL and BnOH. The reaction is shown below.

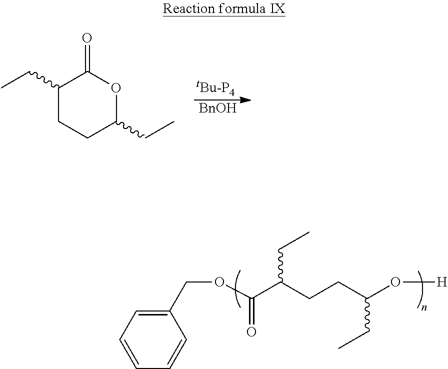

In the following embodiments, the cyclic compound polyHL is prepared by ring-opening polymerization of HL under the catalysis of the organic base. The reaction is as follows.

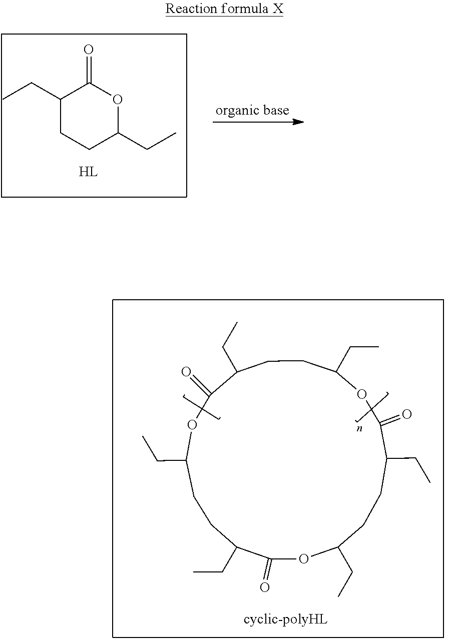

Determination of the number average molecular weight Mₙ and molecular weight distribution Ð of the polymer: taking 200 μL of reaction solution from the reaction system, removing the solvent by rotary evaporation, measure Mₙ (number average molecular weight) and Ð (molecular weight distribution) of the product according to GPC method at 40° C. using tetrahydrofuran as mobile phase and PMMA as standard sample for calibration.

### Embodiment 1˜2M

### Embodiment 1 TBD, DBU-Catalyzed ROP Reaction of HL

### Specific Synthesis Steps:

To achieve the ring-opening polymerization (ROP) of HL to produce polyHL, the initial attempts in the present disclosure involved coordination insertion catalysts, such as tin (II) 2-ethylhexanoate [Sn(Oct)₂] and dibutyltin dilaurate (DBTDL). However, the polymerization was not successful. The organic acid, diphenyl phosphate (DPP), was also used in the present disclosure, but the polymerization was not yet successful. Organic bases such as 1,8-diazabicyclo[5.4.0]undec-7-ene (DBU) and 1,5,7-triazabicyclo[4.4.0]dec-5-ene (TBD) were further tested, and only TBD/BnOH was found to promote moderate monomer conversion, resulting in a lower molecular weight of liquid polyHL (Table 1, run 9: Mₙ=6.0 kg mol⁻¹, Ð=1.14; Table 1, run 10: Mₙ=5.6 kg mol⁻¹, Ð=1.18).

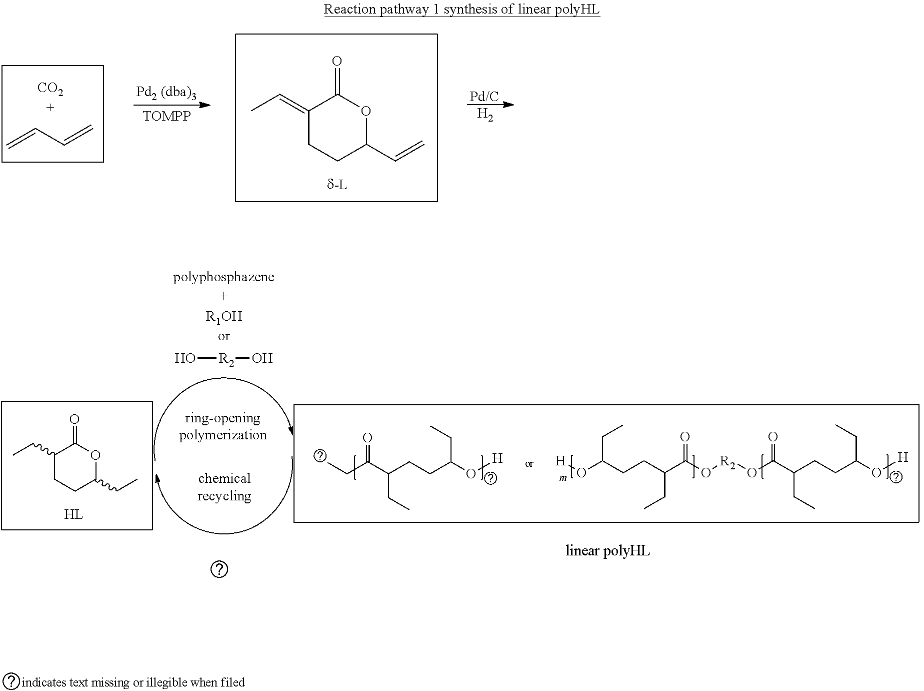

TABLE 1
Initial attempts of ROP of HL

| | HL/ | | | Tem- | | | Con- | Mₙ | |
|---|---|---|---|---|---|---|---|---|---|
| Em- | catalyst./ | | [M]₀ | perature | Time | | version | (kg/ | |
| bodiment | BnOH | Catalyst | (mol/L) | (° C.) | (h) | Solvent | rate (%) | mol) | Ð |
| 1 | 40/1/1 | TBD | 6.3 | 30 | 96 | None | 54 | 6.0 | 1.14 |
| | | | (intrinsic) | | | | | | |
| 2 | 40/1/1 | TBD | 5.0 | 30 | 96 | THF | 37 | 5.6 | 1.18 |

Embodiment 1 The term “intrinsic” represents solvent-free conditions; [M]₀ (mol/L) is the initial concentration of HL monomer in the system, referring to the concentration of the monomer in the system when the reaction is initiated.

### Embodiment 2

In a glove box under a nitrogen atmosphere, 0.0158 mmol of TBD catalyst and 0.0158 mmol of BnOH were added to a flame-dried 10 mL Schlenk tube. The Schlenk tube was then sealed with a rubber stopper, wrapped with a sealing film, taken out of the glove box, and placed in a pre-set 30° C. oil bath until the temperature was stabilized. 0.63 mmol of HL was rapidly injected into the Schlenk tube using a syringe. After stirring for 96 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the purified polyHL sample. Mₙ (number average molecular weight) and Ð (molecular weight distribution) were measured by the GPC method at 40° C., in which tetrahydrofuran is the mobile phase and PMMA is the standard sample for calibration.

### Embodiment 2

The preparation procedure was almost the same as Embodiment 1, except that when adding 0.0158 mmol of TBD catalyst and 0.0158 mmol of BnOH to a flame-dried 10 mL Schlenk tube in a glove box under a nitrogen atmosphere, an additional 0.02 mL of tetrahydrofuran (THF) was required for their dissolution. Please refer to Table 1 for other parameters.

### Embodiments 3˜17 Study of Catalytic Activity of Three Polyphosphazene Combined with BnOH in ROP of HL

When [HL]/[BnOH]=50/1, 1 mol % of ^{t}Bu-P₁ or ^{t}Bu-P₂, −25° C. THE solution ([M]₀=5.3 M) were employed, no polymer was generated under the catalysis of ^{t}Bu-P₁ after 72 h, however, a monomer conversion rate of 41% was achieved under the catalysis of ^{t}Bu-P₂ after five days of reaction. Encouragingly, ^{t}Bu-P₄ greatly facilitated polymer generation with a monomer conversion rate of 87% after 12 h, yielding PolyHL with Mₙ of 19.9 kg mol⁻¹ and a slightly broad molecular weight distribution Ð of 1.90. The pKa of ^{t}Bu-P₁, ^{t}Bu-P₂, and ^{t}Bu-P₄ in acetonitrile were 26.9, 33.5, and 42.7, respectively, which might be caused by the large alkalinity difference among the three polyphosphazenes. Then, the loading of ^{t}Bu-P₄ was gradually decreased from 2 mol % to 0.2 mol %, to make the polymerization more controllable. In particular, when the loading of ^{t}Bu-P₄ was 0.2 mol %, polyHL with Mₙ of 9.2 kg mol⁻¹ and a very narrow dispersion Ð of 1.09 was obtained (Table 2, run 7), indicating living ring-opening polymerization (ROP) of HL. However, as the initial monomers of the system were gradually diluted in THF to a concentration of 2.0, 1.6, and 1.3 M, the ROP of HL became less controllable: the conversion decreased significantly, and Mₙ also decreased (Table 2, run 10-12). Increasing the reaction temperature from −25° C. to 41° C. also resulted in a more uncontrollable polymerization. Two sets of experiments with [HL]/[BnOH] ratios of 25/1 and 100/1 were further designed in the present disclosure. Under both polymerization conditions, maximum monomer conversion rates of 88% were achieved. The Mₙ of the resulting polymers increased linearly with an increasing [HL]/[BnOH] ratio, while the distribution of the polymers remained consistently narrow.

TABLE 2
Reaction results of ROP of HL catalyzed by polyphosphazene/BnOH

| | HL/ | | Tem- | | [M]₀ | Conversion | Mₙ | |
|---|---|---|---|---|---|---|---|---|
| Em- | catalyst/ | | perature | Time | (mol | rate | (kg | |
| bodiment | BnOH | Catalyst | (° C.) | (h) | L⁻¹) | (%) | mol⁻¹) | Ð |
| 3 | 50/0.5/1 | ^{t}Bu-P₁ | −25 | 72 | 5.3 | n.d. | — | — |
| 4 | 50/0.5/1 | ^{t}Bu-P₂ | −25 | 120 | 5.3 | 41 | 3.7 | 1.16 |
| 5 | 50/0.5/1 | ^{t}Bu-P₄ | −25 | 12 | 5.3 | 88 | 18.9 | 1.69 |
| 6 | 50/1/1 | ^{t}Bu-P₄ | −25 | 12 | 5.3 | 87 | 19.9 | 1.90 |
| 7 | 50/0.25/1 | ^{t}Bu-P₄ | −25 | 12 | 5.3 | 88 | 13.1 | 1.35 |
| 8 | 50/0.2/1 | ^{t}Bu-P₄ | −25 | 12 | 5.3 | 88 | 12.9 | 1.23 |
| 9 | 50/0.1/1 | ^{t}Bu-P₄ | −25 | 8 | 5.3 | 88 | 9.2 | 1.09 |
| 10 | 50/0.1/1 | ^{t}Bu-P₄ | −25 | 12 | 2.0 | 67 | 6.9 | 1.74 |
| 11 | 50/0.1/1 | ^{t}Bu-P₄ | −25 | 12 | 1.6 | 58 | 6.8 | 1.39 |
| 12 | 50/0.1/1 | ^{t}Bu-P₄ | −25 | 12 | 1.3 | 37 | 4.7 | 1.60 |
| 13 | 50/0.1/1 | ^{t}Bu-P₄ | −9 | 12 | 5.3 | 82 | 10.3 | 1.64 |
| 14 | 50/0.1/1 | ^{t}Bu-P₄ | 28 | 12 | 5.3 | 65 | 5.7 | 2.28 |
| 15 | 50/0.1/1 | ^{t}Bu-P₄ | 41 | 12 | 5.3 | 52 | 5.5 | 2.18 |
| 16 | 25/0.1/1 | ^{t}Bu-P₄ | −25 | 12 | 5.3 | 87 | 4.1 | 1.11 |
| 17 | 100/0.2/1 | ^{t}Bu-P₄ | −25 | 12 | 5.3 | 88 | 19.6 | 1.08 |

[M]₀ (mol/L) is the initial concentration of HL monomer in the system, referring to the concentration of the monomer in the system when the reaction is initiated.

### Embodiment 3

In a glove box under nitrogen atmosphere, 0.0063 mmol of ^{t}Bu-P₁ catalyst and 0.0126 mmol of BnOH were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature was stabilized. 0.63 mmol of HL was rapidly injected into the Schlenk tube using a syringe. After stirring for 72 h, 1 mL of 5% HCl-methanol solution was added to quench the reaction. 50 μL of the reaction solution was taken out for ¹H NMR analysis to determine the monomer conversion rate.

### Embodiment 4

In a glove box under nitrogen atmosphere, 0.0063 mmol of ^{t}Bu-P₂ catalyst and 0.0126 mmol of BnOH were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature was stabilized. 0.63 mmol of HL was rapidly injected into the Schlenk tube using a syringe. After stirring for 120 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the final product. Mₙ (number average molecular weight) and Ð (molecular weight distribution) were measured by the GPC method at 40° C., in which tetrahydrofuran is the mobile phase and PMMA is the standard sample for calibration.

### Embodiments 5-9

The preparation procedure was almost the same as Embodiment 4, except that the molar concentration of the catalyst was adjusted in the range of 0.0126˜0.126 mmol. Please refer to Table 2 for other parameters. The characterization of the polymer sample obtained in Embodiment 9 is shown in Embodiment 19.

### Embodiments 10-12

The preparation procedure was almost the same as Embodiment 9, except that the initial molar concentration of HL in the system was gradually reduced from 5.3 M to 1.3 M. Please refer to Table 2 for other parameters.

### Embodiments 13-15

The preparation procedure was almost the same as Embodiment 9, except that the reaction temperature was increased from −25° C. to −9° C., 28° C., and 41° C., respectively. Please refer to Table 2 for other parameters.

### Embodiments 16-17

The preparation procedure was almost the same as Embodiment 9, except that the feeding ratios of HL, ^{t}Bu-P₄, and BnOH were adjusted to 25/0.1/1 and 100/0.2/1, respectively. Please refer to Table 2 for other parameters. The characterization of the polymer sample obtained in Embodiment 16 is shown in Embodiment 19.

### Embodiment 18: Synthesis of Polyester Diols Using 1,3-Propylene Glycol as Initiator

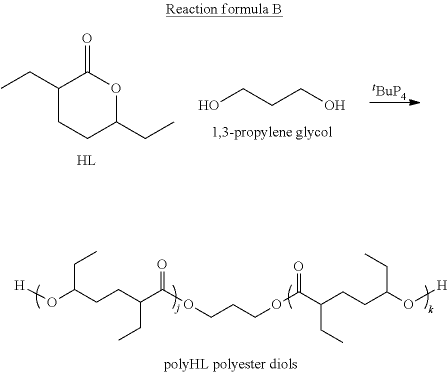

In the above formula, j and k are positive integers greater than 1, and j and k may be the same or different.

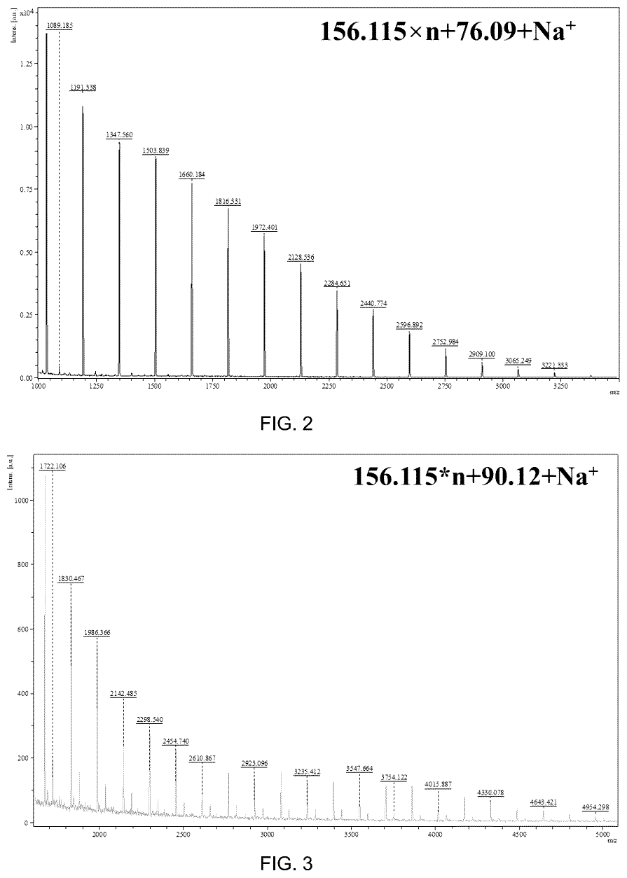

Synthesis steps: in a glove box under nitrogen atmosphere, 0.0063 mmol of ^{t}Bu-P₄ catalyst and 0.042 mmol of 1,3-propylene glycol were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature was stabilized. 0.63 mmol of HL was rapidly injected into the Schlenk tube using a syringe. After stirring for 48 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the final product. FIG. 2 shows MALDI-TOF of the product.

### Embodiment 19: Synthesis of Polyester Diols Using 1,4-Butanediol as Initiator

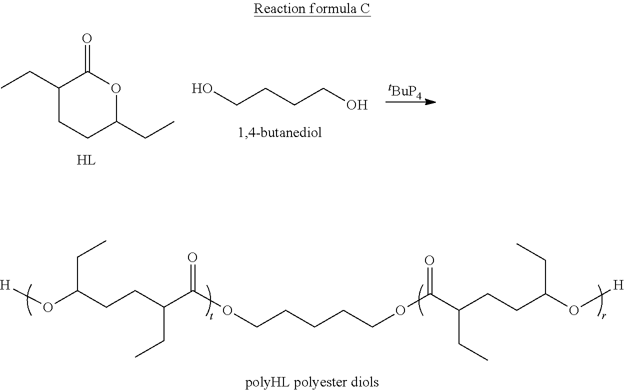

In the above formula, t and r are positive integers greater than 1, and t and r may be the same or different.

Synthesis steps: in a glove box under nitrogen atmosphere, 0.0063 mmol of ^{t}Bu-P₄ catalyst and 0.042 mmol of 1,4-butanediol were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature was stabilized. 0.63 mmol of HL was rapidly injected into the Schlenk tube using a syringe. After stirring for 48 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the final product. FIG. 3 shows MALDI-TOF of the product.

### Embodiment 20 Characterization of the Ring-Opening Polymerization ROP Reaction in Embodiment 9

The reaction system, reaction conditions, reaction steps, etc. of the ring-opening polymerization reaction, as detailed in Embodiment 9, were kept consistent, and only the reaction time was varied. Several parallel polymerization reactions were carried out to study how the conversion rate evolves over time. These reactions were quenched after a predetermined time to avoid systematic errors in conversion caused by constantly sampling from the same reaction system.

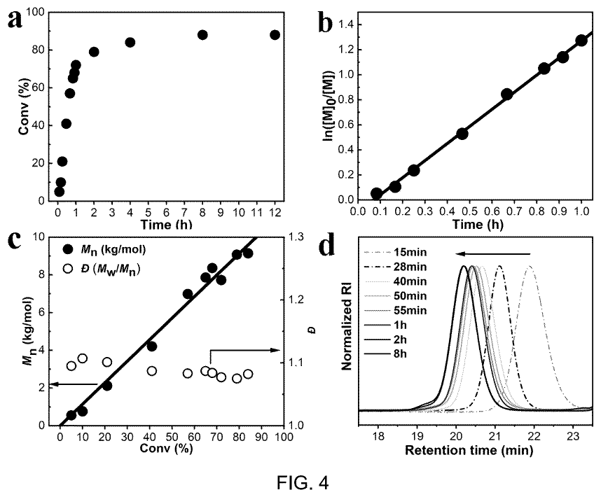

The data in FIGS. 4a and 4b are derived from conversion rates obtained by 1H NMR, and the data in FIGS. 4c and 4d are derived from GPC analysis. The polymerization kinetics data indicated that the polymerization under these conditions is living polymerization. The monomer conversion rate increased linearly with reaction time (FIG. 4a), and the curve of In[M]₀/[M] versus time exhibited very clear first-order kinetic characteristic (FIG. 4b). The Mₙ of polyHL was linearly correlated with the monomer conversion rate, and the distribution of the resulting polyHL remained narrow (Ð˜1.1) during the chain growth phase (FIG. 4C). The GPC curves with unimodal distribution given in FIG. 4d also confirmed this conclusion.

### Embodiment 21 Characterization of the Polymer Molecules Obtained in Embodiments 9 and 16

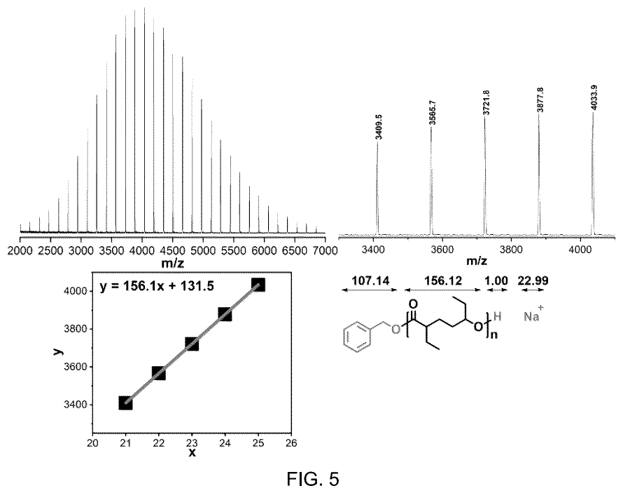

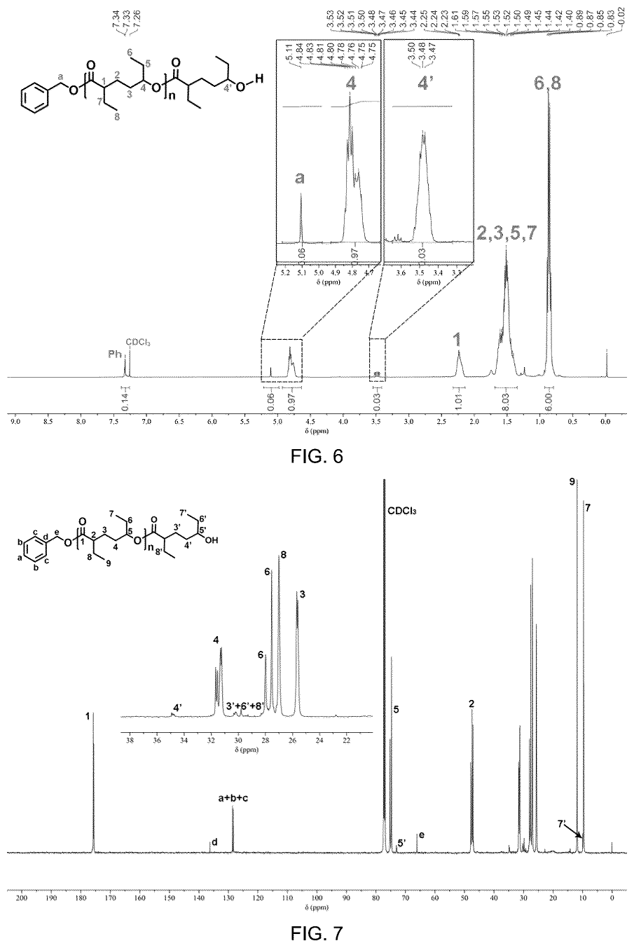

The chain end group fidelity of the synthesized polyester (polyHL) was investigated using matrix-assisted laser desorption ionization time-of-flight mass spectrometry (MALDI-TOF MS). The MS signals of polyHL showed only one set of molecular ion peaks, which is consistent with the structure of linear polyHL initiated by BnOH as expected in the present disclosure (FIG. 5). The polyHL catalyzed by ^{t}Bu-P₄/BnOH system was further subjected to NMR spectroscopy for its structure, and proton signal of the methylene group on BnO— (δ 5.12 ppm) and proton signal of the methenyl group attached to the hydroxyl end group (δ 3.49 ppm) were observed in the ¹H NMR spectra (FIG. 6). These proton signals were also observed correspondingly in ¹³C NMR spectra (FIG. 7).

MALDI-TOF experimental procedure: The selected samples were the purified polymer in Embodiment 9. The samples were tested using a Bruker Autoflex Speed MALDI-TOF mass spectrometer in positive ion and reflectron mode. A drop of 1% NaI solution was added on top surface of a stainless steel target plate, after which 1 μL of a mixture of matrix and polymer sample was added thereon (DHB is the matrix, specifically, DHB is 2,5-dihydroxybenzoic acid and has a concentration of 20 mg mL⁻¹ in THF). A peptide calibration standard sample was additionally placed next to the sample wells on the target plate to perform external calibration of the molecular weight. Raw data was processed by FlexAnalysis software and used for the plotting of spectra shown in FIG. 5. The drawings were all plotted by Origin Pro 2019b.

The samples in Embodiment 16 were subjected to NMR, and the results were shown in FIGS. 6 and 7. Since the BnOH-initiated polymers have identical chemical structures, the difference in molecular weight has no effect on the chemical shifts and characteristic peaks in the NMR spectra, and there is only a difference in the integral area of the polymer end groups. The characteristic spectra of the polymers obtained from the preferred conditions in the embodiments was illustrated herein as representative.

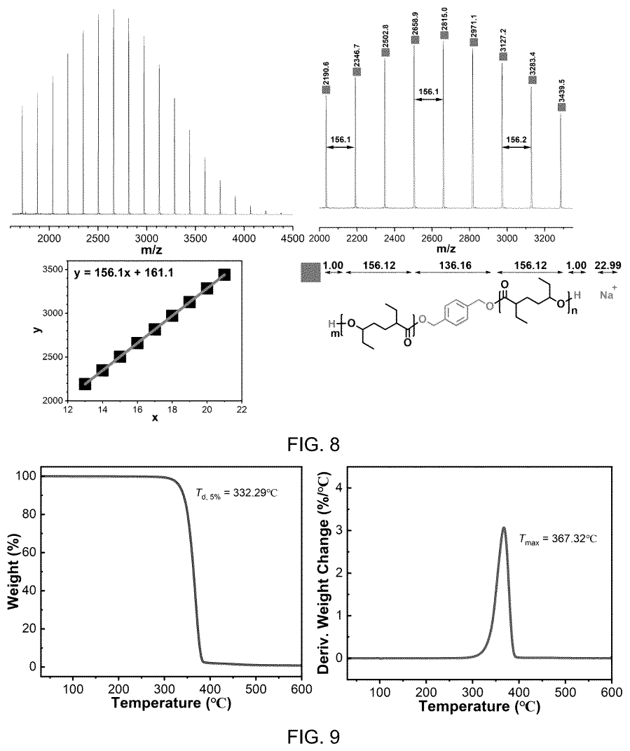

Telechelic polymer polyHL (with dihydroxy end-capped) having good chain end fidelity was synthesized using ^{t}Bu-P₄ and 1,4-benzenedimethanol (1,4-BDM) as catalysts at [HL]/[^{t}Bu-P₄]/[1,4-BDM]=15/0.15/1. The MALDI-TOF MS signal showed a set of signal peaks of Mₙ=n×156.1+161.1, matching with the expected diol end-capped structure (FIG. 8).

### Embodiment 22 Preparation of a Linear polyHL Diol Sample Using 1,4-Benzenedimethanol

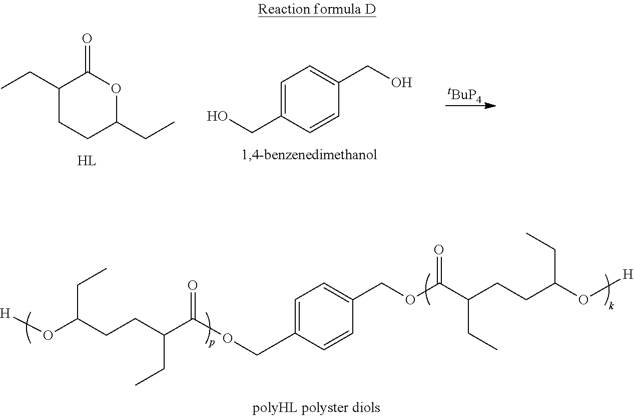

In the above formula, p and q are positive integers greater than 1, and p and q may be the same or different.

In a glove box under nitrogen atmosphere, 0.0063 mmol of ^{t}Bu-P₄ catalyst and 0.042 mmol of 1,4-BDM were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature stabilized. 0.63 mmol of HL was rapidly injected into the Schlenk tube using a syringe. After stirring for 48 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the final product.

### Embodiment 23 Determination of Physical Properties of polyHL

The thermal stability of polyHL prepared under the ^{t}Bu-P₄/BnOH system in Embodiment 17 was investigated by thermogravimetric analysis (TGA) and differential scanning calorimetry (DSC).

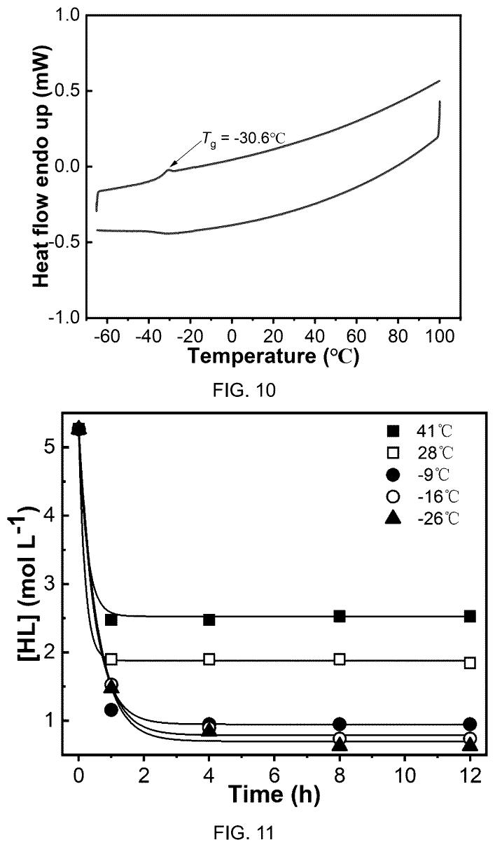

The linear polyHL exhibited excellent thermal stability: TGA and DTG results showed that T_{d,5%}=326.0° C., Tₘₐₓ=335.3° C. (FIG. 9). The DSC curve of the second heating and scanning (5° C. min-1) of the linear polyHL sample showed a glass transition temperature (Tg) of −30.6° C. and no crystallization peak (FIG. 10). The results indicated that polyHL is an amorphous polymer material with good thermal stability.

### Embodiment 24 Determination of Chemical Recyclability of PolyHL in Solvent

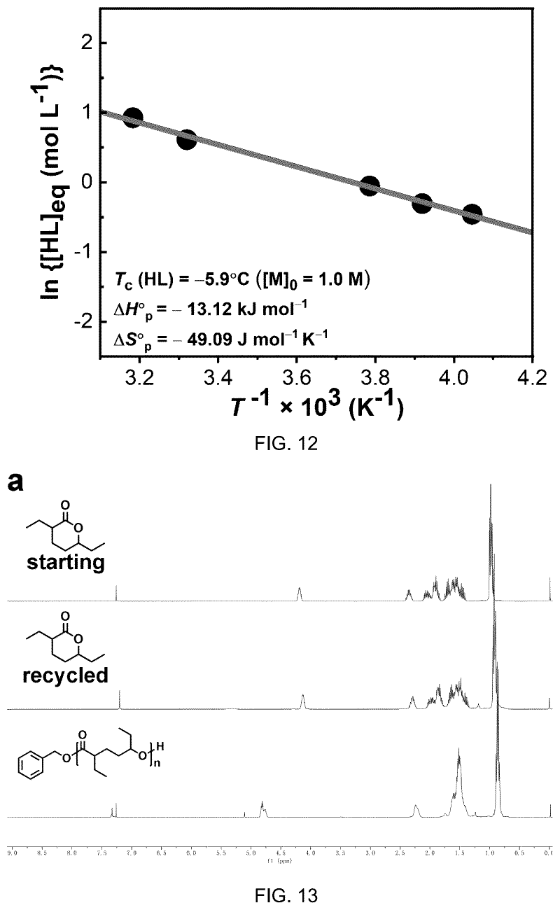

Sample preparation for thermodynamic experiments: the experimental conditions for preparing the PolyHL samples were almost the same as those in Embodiment 9, only the reaction temperatures were adjusted to −26° C., −16° C., −9° C., 28° C., and 41° C., respectively, and the reaction time was changed accordingly, so as to measure the conversion rate at different time. FIG. 11 shows HL monomer concentration versus time under different temperatures. FIG. 12 shows the analyzed Van′t Hoff curve for HL.

The ROP reaction of HL catalyzed by ^{t}BuP₄/BnOH was further investigated at different temperatures (−25, −16, −9, 28, and 41° C.) in the present disclosure, and polymerization thermodynamic parameters were calculated (FIGS. 11 and 12). The enthalpy change (ΔH°ₚ) and entropy change (ΔS°ₚ) of THF at [HL]₀=1.0 mol L⁻¹ were calculated to be −13.12 kJ mol⁻¹ and −49.09 J mol⁻¹ K⁻¹, respectively, according to the Dainton equation, and T_{c} was further calculated to be −6° C. Compared to other monosubstituted and unsubstituted six-membered cyclic lactones, this Tc is significantly lower. Thus, the introduction of substituents at the α- and δ- positions of δ-VL is effective in chemically recycling the monomer.

### Embodiment 25 Determination of Chemical Recyclability of PolyHL in Solvent

Chemical recycling experiment: in a glove box under nitrogen atmosphere, 200 mg of polyHL (Embodiment 9) and 5 mol % zinc chloride were added to a 10 mL Schlenk tube, and 2.6 mL of ortho-dichlorobenzene (o-DCB) was added for their dissolution. The Schlenk tube was then sealed with a rubber stopper, wrapped with a sealing film, taken out of the glove box, and placed in a pre-set 160° C. oil bath with heating and stirring for 12 h. The solvent in the system was removed, and 10 mg of the degraded liquid product was subjected to ¹H NMR for the HL yield.

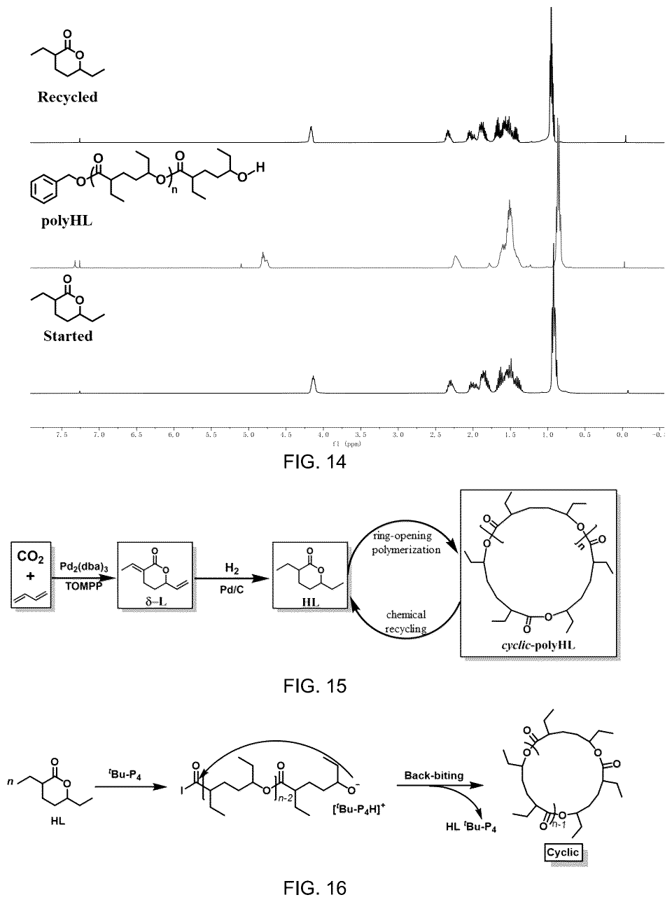

The recycling results were shown in FIG. 13, it can be seen that the HL yield reached 100%, indicating that the ZnCl₂ as a catalyst is effective for the chemical recycling of linear polyHL.

### Embodiment 26 Determination of Chemical Recyclability of PolyHL Using Thermolysis Method Under Solvent-Free Conditions

In a glove box under nitrogen atmosphere, 200 mg of polyHL sample (Embodiment 9) was added to a 10 mL Schlenk tube. The Schlenk tube was then sealed with a rubber stopper, wrapped with a sealing film, taken out of the glove box, and placed in a pre-set 220° C. sand bath with heating and stirring for 3 h and evacuated continuously using a rotary vane vacuum pump. After 3 h, a colorless oily liquid was obtained. 10 mg of this liquid was subjected to ¹H NMR analysis for the HL yield.

The recycling results were shown in FIG. 14, it can be seen that the HL yield reached 100%, indicating that thermolysis is effective for the chemical recycling of polyHL under solvent-free conditions.

### Embodiments 27˜32: Ring-Opening Polymerization of HL Catalyzed by ^{t}BuP₄

In the present disclosure, three commonly used phosphazene bases: ^{t}Bu-P₁, ^{t}Bu-P₂, and ^{t}Bu-P₄ were selected as catalysts, the molar ratio of [HL]/[phosphazene base] was controlled to be 50/1, and the reaction was carried out in tetrahydrofuran (THF) at −25° C. for 12 h. The results were shown in Table 1. After 12 h of reaction, no polymer was produced under the catalysis of either ^{t}Bu-P₁ or ^{t}Bu-P₂ (Embodiment 27, Embodiment 28). Surprisingly, after 12 h, a maximum conversion rate of 88% was observed under the catalysis of ^{t}Bu-P₄ (Table 1), and a polyHL sample with an ultra-high molecular weight Mₙ=613.8 kg mol1 and a medium molecular weight distribution Ð=1.45 was obtained (Embodiment 29). It was further found that varying the concentration of ^{t}Bu-P₄ in the system had only a slight effect on the molecular weight Mₙ and molecular weight distribution Ð of the resulting polyHL (Embodiments 30-32).

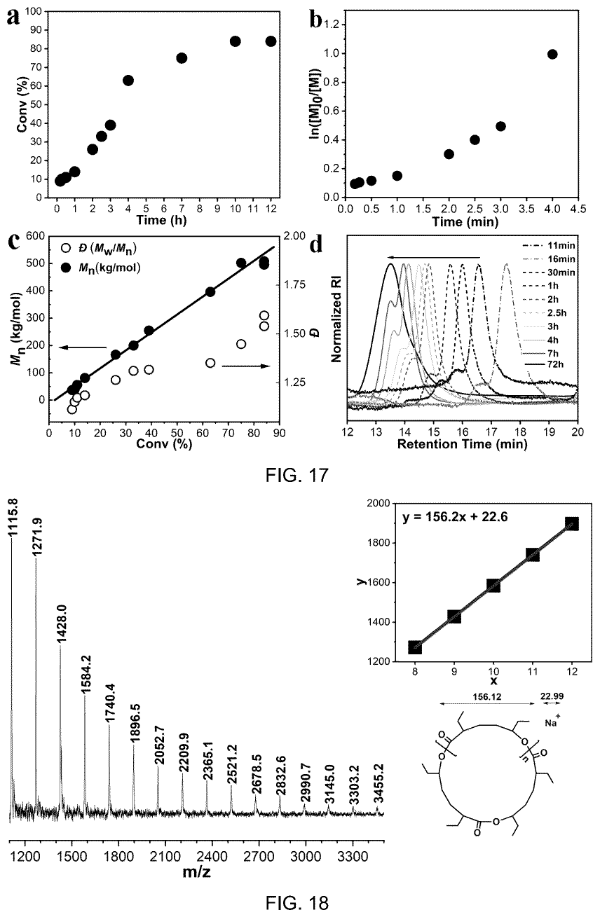

To determine the controllability of the ROP of HL catalyzed by ^{t}Bu-P₄ alone, kinetic experiments were carried out in THE solution ([HL]/[^{t}Bu-P₄] molar ratio=50/1, [M]₀=4.0 M) at 25° C. (FIG. 17 and Table 4). The In[M]₀/[M] versus time curve showed that the conversion rate reached 63% at 4 h, after which the polymerization rate tended to slow down and reached 84% at 6-8 h (FIG. 17a). The curve of In[M]₀/[M] versus time exhibited very clear first-order kinetic characteristic (FIG. 17b). Interestingly, the Mₙ of polyHL is linearly correlated with the monomer conversion rate, and the molecular weight distribution was broadened significantly when the monomer conversion rate was higher than 50% (FIG. 17c). The GPC curves also gradually showed a bimodal distribution as the reaction time increased, possibly due to severe ester exchange reactions occurring at high monomer conversion rates (FIG. 17d). These data suggested that if the reaction was quenched within 4 h, the ROP was relatively controllable and high molecular weight polymers can be produced.

TABLE 3
Experimental results of ring-opening polymerization of
HL monomers under the catalysis of phosphazene bases

| | | | Conversion | Mₙ | |
|---|---|---|---|---|---|
| Embodiment | HL/catalyst | Catalyst | rate (%) | (kg/mol) | Ð |
| 1 | 50/1 | ^{t}Bu—P₁ | n.d. | — | — |
| 2 | 50/1 | ^{t}Bu—P₂ | n.d. | — | — |
| 3 | 50/1 | ^{t}Bu—P₄ | 88 | 613.8 | 1.45 |
| 4 | 100/1 | ^{t}Bu—P₄ | 87 | 571.5 | 1.40 |
| 5 | 150/1 | ^{t}Bu—P₄ | 87 | 543.4 | 1.40 |
| 6 | 200/1 | ^{t}Bu—P₄ | 72 | 598.8 | 1.35 |

TABLE 4
Kinetic experimental data of ring-opening
polymerization catalyzed by phosphazene bases

| | | | Con- | | | | | |
|---|---|---|---|---|---|---|---|---|
| Em- | | | version | Mₙ | | [M]ₜ | ln([M]₀/ | [M]₀/ |
| bodiment | Time | | rate (%) | (kg/mol) | Ð | (mol/L) | [M]ₜ) | [M]ₜ |
| 7 | 0.183 | h | 9 | 36.5 | 1.11 | 3.64 | 0.094311 | 1.098901 |
| | (11 | min) | | | | | | |
| 8 | 0.267 | | 10 | 39.2 | 1.15 | 3.60 | 0.105361 | 1.111111 |
| | (16 | min) | | | | | | |
| 9 | 0.5 | h | 11 | 55.5 | 1.17 | 3.56 | 0.116534 | 1.123596 |
| 10 | 1 | h | 14 | 80.8 | 1.19 | 3.44 | 0.150823 | 1.162791 |
| 11 | 2 | h | 26 | 166.3 | 1.26 | 2.96 | 0.301105 | 1.351351 |
| 12 | 2.5 | h | 33 | 199.6 | 1.31 | 2.68 | 0.400478 | 1.492537 |
| 13 | 3 | h | 39 | 254.5 | 1.32 | 2.44 | 0.494296 | 1.639344 |
| 14 | 4 | h | 63 | 396.2 | 1.35 | 1.48 | 0.994252 | 2.702703 |
| 15 | 7 | h | 75 | 502.2 | 1.45 | 1.00 | 1.386294 | 4 |
| 16 | 10 | h | 84 | 507.8 | 1.54 | 0.64 | 1.832581 | 6.25 |
| 17 | 12 | h | 84 | 495.7 | 1.59 | 0.64 | 1.832581 | 6.25 |

### Embodiment 27

In a glove box under nitrogen atmosphere, 0.0126 mmol of ^{t}Bu-P₁ catalyst was added to a flame-dried 10 mL Schlenk tube, and then 0.025 mL of tetrahydrofuran was added to dissolve the ^{t}Bu-P₁. The Schlenk tube was then sealed with a rubber stopper, wrapped with a sealing film, taken out of the glove box, and placed in a pre-set −25° C. low temperature bath until the temperature stabilized. 0.63 mmol of HL was rapidly injected into the Schlenk tube using a syringe. After stirring for 12 h, 1 mL of 5% HCl-methanol solution was added to quench the reaction. 50 μL of the reaction solution was taken out for ¹H NMR analysis to determine the monomer conversion rate. The results were shown in Table 3.

### Embodiment 28

The preparation procedures were almost the same as those in Embodiment 27, except that the catalyst was replaced with an equal equivalent amount of ^{t}Bu-P₂.

### Embodiment 29

The preparation procedures were almost the same as those in Embodiment 27, except that the catalyst was replaced with an equal equivalent amount of ^{t}Bu-P₄. The specific steps were shown as follows.

In a glove box under nitrogen atmosphere, 0.0126 mmol of ^{t}Bu-P₄ catalyst was added to a flame-dried 10 mL Schlenk tube, and then 0.025 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 0.63 mmol of HL was rapidly injected into the Schlenk tube using a syringe. After stirring for 12 h, 1 mL of 5% HCl-methanol solution was added to quench the reaction. 50 μL of the reaction solution was taken out for ¹H NMR analysis to determine the monomer conversion rate.

The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the purified PolyHL sample. The Mₙ (number average molecular weight) and Ð (molecular weight distribution) of the product were measured.

### Embodiments 30˜32

The preparation procedures were almost the same as those in Embodiment 29, except that the catalyst equivalent amount was adjusted from 2 mol % to 1 mol % (Embodiment 30), 0.67 mol % (Embodiment 31, where HL/catalyst was 150/1), and 0.5 mol % (Embodiment 32), respectively, as detailed in Table 3.

### Embodiments 33˜43: Kinetic Study of Ring Opening Polymerization of HL Under the Catalysis of ^{t}Bu-P₄

### Polymer Preparation Method in Embodiment 33

In a glove box under nitrogen atmosphere, 0.0126 mmol of ^{t}Bu-P₄ catalyst was added to a flame-dried 10 mL Schlenk tube, and then 0.058 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 0.63 mmol of HL was rapidly injected into the Schlenk tube using a syringe. After stirring for 11 min, 1 mL of 5% HCl-methanol solution was added to quench the reaction. 50 μL of the reaction solution was taken out for ¹H NMR analysis to determine the monomer conversion rate. The Mₙ (number average molecular weight) and Ð (molecular weight distribution) of the product were measured.

### Polymer Preparation Method in Embodiments 34-43

The preparation procedures were almost the same as those in Embodiment 33, except that the stirring reaction time was adjusted from 11 min to about 0.267 h (i.e., 16 min) (Embodiment 34), 0.5 h (Embodiment 35), 1 h (Embodiment 36), 2 h (Embodiment 37), 2.5 h (Embodiment 38), 3 h (Embodiment 39), 4 h (Embodiment 40), 7 h (Embodiment 41), 10 h (Embodiment 42), and 12 h (Embodiment 43), respectively.

### Embodiment 44 MALDI-TOF Spectra Characterization of the Synthesized Cyclic Polymers when Residual Water was Strictly Removed from HL

### (1.1) Sample Preparation

In a glove box under nitrogen atmosphere, 0.0126 mmol of ^{t}Bu-P₄ catalyst was added to a flame-dried 10 mL Schlenk tube, and then 0.058 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 0.63 mmol of HL was rapidly injected into the Schlenk tube using a syringe. After stirring for 3-5 min, 1 mL of 5% HCl-methanol solution was added to quench the reaction. The quenched reaction solution was added dropwise to 20 mL of ice methanol, the supernatant was discarded after centrifugation, and the precipitate was the sample to be tested.

### (1.2) MALDI-TOF Spectra Characterization

The sample to be tested prepared in step (1.1) of this embodiment was dried in a vacuum drying oven until its weight was constant, and a small amount of the sample was taken out for MALDI-TOF analysis. The results were shown in FIG. 18, and only one set of signals attributable to the mass peaks of cyclic polyHL was observed.

(1.3) ¹H and ¹³C NMR Spectra Characterization

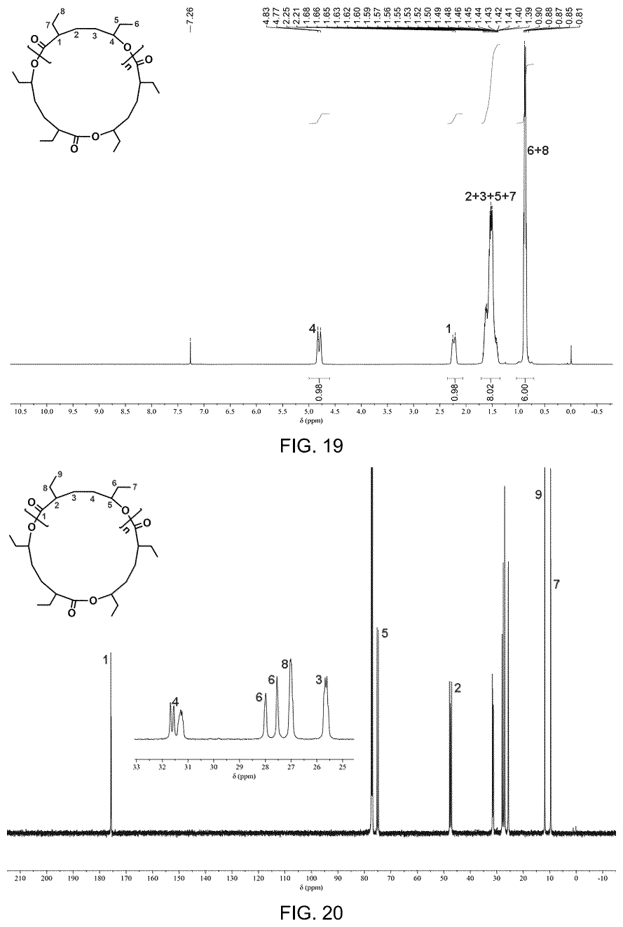

The cyclic polymer sample prepared in Embodiment 29 was subjected to the 1H and ¹³C NMR, and the results showed no end group signals in the spectra, supporting that the polymer has a cyclic structure (FIGS. 19 and 20).

### Embodiment 45 MALDI-TOF Spectra Characterization of the Synthesized Cyclic Polymers when Residual Water was not Strictly Removed from HL

### (1.1) Sample Preparation

The preparation procedures were almost the same as those in Embodiment 44, except that the used HL monomer was not rigorously dewatered, and the water content of the HL monomer was approximately 100 ppm as measured by a Karl Fischer moisture meter.

### (1.2) MALDI-TOF Spectra Characterization

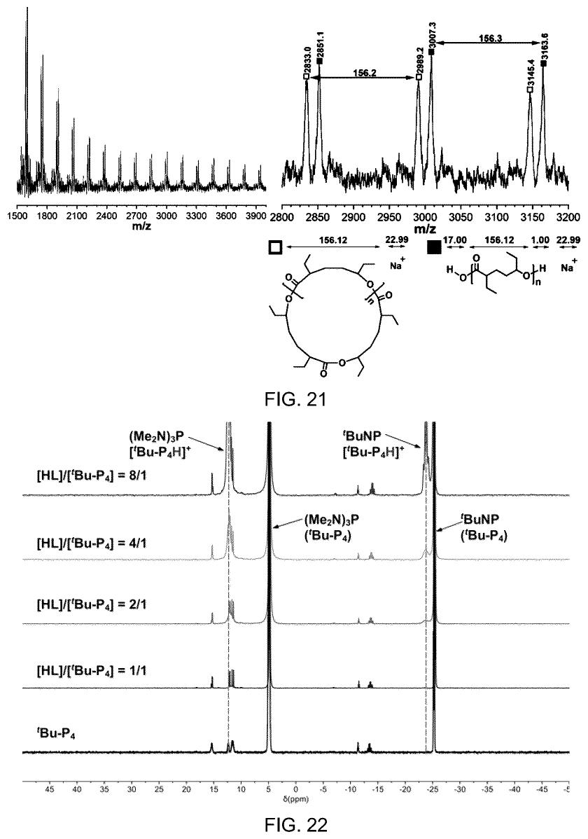

The samples to be tested were dried in a vacuum drying oven until its weight was constant, and a small amount of the sample was taken out for MALDI-TOF analysis. The results were shown in FIG. 21. From the figure, it can be seen that only when the residual water in the HL monomer or preparation system is not strictly removed, it is possible to detect the linear mass peaks initiated by water.

### Embodiment 46 Possible Chain Initiation Mechanism of the ^{t}Bu-P₄ Alone Catalyzed ROP of HL Leading to the Formation of Cyclic polyHL Products

The ability of the catalyst ^{t}Bu-P₄ to extract acidic H from HL was verified by ¹H NMR spectroscopy. HL and ^{t}Bu-P₄ were mixed at room temperature in molar ratios of 1/1, 2/1, 4/1, and 8/1, respectively, and their reactions were monitored by the 1H and 31p NMR spectra.

The results were shown in FIGS. 22-24, it can be seen that the characteristic signals of [^{t}Bu-P₄H] were δ 7.92-8.07 ppm in ¹H NMR, δ 12.48 ppm and −23.64 ppm in ³¹P NMR. The intensity of the [^{t}Bu-P₄H] signal increased slightly with increasing HL/^{t}Bu-P₄ ratio. The characteristic signals of ^{t}Bu-P₄ were: δ 1.72, 2.70, and 2.72 ppm in ¹H NMR, and δ 4.96 and −25.31 ppm in ³¹P NMR. These data clearly indicated that ^{t}Bu-P₄ can deprotonate a very small fraction of HL monomers, which may explain the lack of a direct correlation between the ^{t}Bu-P₄ concentration and the Mₙ of the cyclic polyHL synthesized by the preparation system of the present disclosure (see the description above).

HL and ^{t}Bu-P₄ Mixing Experiment at Different Ratios (³¹P NMR Spectra)

Experimental procedure: in a glove box under nitrogen atmosphere, an equivalent amount of ^{t}Bu-P₄ catalyst as shown in FIG. 22 was added to 0.6 mL of dry deuterotoluene for its dissolution. A defined equivalent amount of HL monomer was added to the solution, and after stirring, the mixture was transferred to a flame-dried J-Young NMR tube and immediately characterized by NMR spectroscopy. The results were shown in FIG. 22.

HL and ^{t}Bu-P₄ Mixing Experiment at Different Ratios (¹H NMR Spectra)

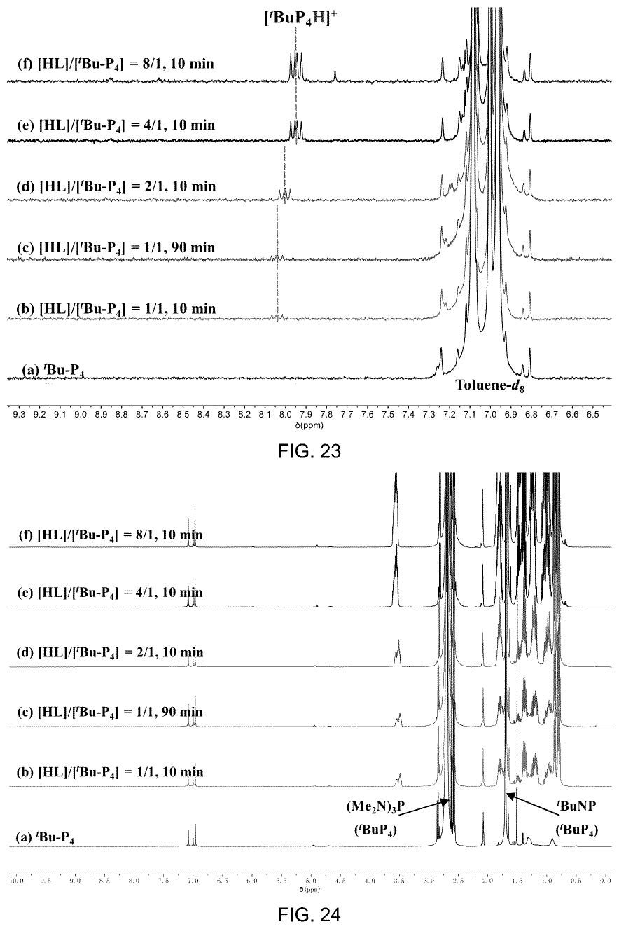

Experimental procedure: in a glove box under nitrogen atmosphere, an equivalent amount of ^{t}Bu-P₄ catalyst as shown in FIGS. 23-24 was added to 0.6 mL of dry deuterotoluene for its dissolution. A defined equivalent amount of HL monomer was added to the solution, and after stirring for the time indicated in the figure, the mixture was transferred as a flame-dried J-Young NMR tube and immediately characterized by NMR spectroscopy. The results were shown in FIGS. 23-24.

### Embodiment 47 the Gibbs Free Energy Differences for ^{t}Bu-P₄ to Extract Protons from BnOH, HL, and H₂O

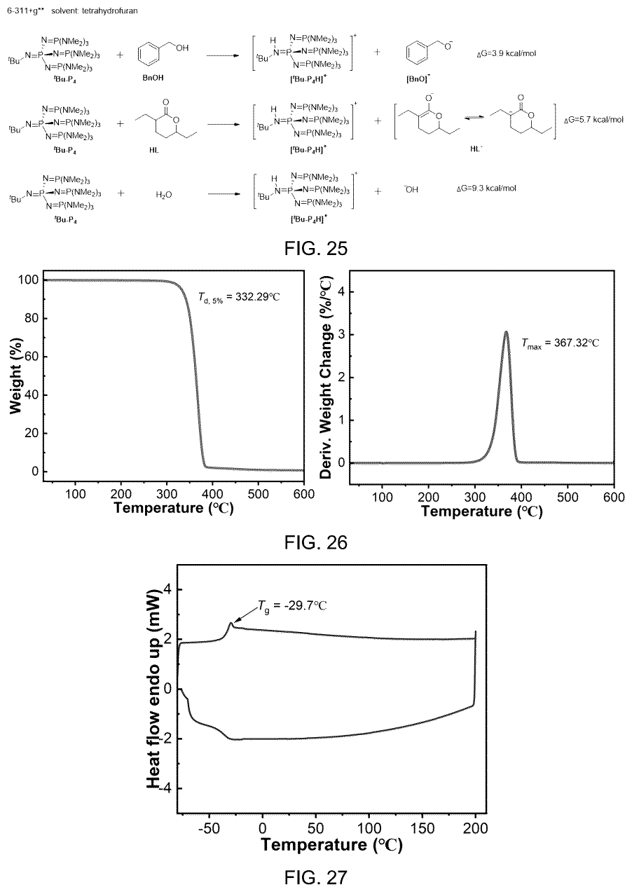

The present disclosure evaluated, through quantum mechanical calculations, the Gibbs free energy associated with ^{t}Bu-P₄ abstracting a proton from the HL monomer to form a cyclic polymer, and compared this with the Gibbs free energy of ^{t}Bu-P₄ abstracting a proton from BnOH and water molecules. These calculations were used to verify the feasibility of the proton abstraction process leading to the formation of cyclic polyHL. The reaction process and free energy data were shown in FIG. 25. The Gibbs free energy for the proton extraction reactions followed the order of BnOH (3.9 kcal mol⁻¹)<HL (5.7 kcal mol⁻¹)<H₂O (9.3 kcal mol⁻¹), indicating that BnOH is the most favorable initiator for generating initiating active species. The energy required for HL to generate initiating active species is only 1.8 kcal mol⁻¹ higher than that of BnOH, indicating that HL can generate cyclic polyHL under the catalysis of ^{t}Bu-P₄ through a proton extraction mechanism.

### Embodiment 48 Physical Properties of Cyclic polyHL

### (1) Sample Preparation

In a glove box under nitrogen atmosphere, 0.063 mmol of ^{t}Bu-P₄ catalyst was added to a flame-dried 10 mL Schlenk tube, and then 0.058 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 0.63 mmol of HL was rapidly injected into the Schlenk tube using a syringe. After stirring for 30 min, 1 mL of 5% HCl-methanol solution was added to quench the reaction. The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the purified polyHL sample. The Mₙ (number average molecular weight) and Ð (molecular weight distribution) of the product were measured. The results showed that Mₙ 21.7 kg/mol and Ð)=1.13.

(2) Thermal Stability Analysis of Cyclic polyHL Prepared by ^{t}Bu-P₄ System Using Thermogravimetric Analysis (TGA), Differential Thermogravimetric Analysis (DTG), and Differential Scanning Calorimetry (DSC)

The results were shown in FIGS. 26-27. As can be seen from FIG. 26, the cyclic polyHL exhibited excellent thermal stability (Td,5%>325° C.). The TGA and DTG curves of the product showed that T_{d,5%}=332.3° C. and Tₘₐₓ=367.3° C. As can be seen from FIG. 27, the second heating cycle (5° C. min-1) of the cyclic polyHL sample showed a glass transition temperature (Tg) of −29.7° C. and no crystallization peak.

The results indicated that polyHL is an amorphous polymer material with good thermal stability.

### Embodiment 49 Determination of Peel Properties of polyHL Polymers with Different Molecular Weights

### (1) Preparation of Cyclic Polymer Sample

Ring-opening polymerization of HL catalyzed by ^{t}Bu-P₄ alone can produce polyHL with high molecular weight, therefore, it is very promising to obtain pressure-sensitive adhesives with potential application values. In this embodiment, the peel strength of polyHL with different molecular weights was tested using a simple 180° peel experiment, and the procedures were as follows.

Preparation of polyHL_160 (molecular weight Mₙ is 160) cyclic polymer sample: in a glove box under nitrogen atmosphere, 0.025 mmol of ^{t}Bu-P₄ catalyst was added to a flame-dried 10 mL Schlenk tube, and then 0.025 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 1.26 mmol of HL was rapidly injected into the Schlenk tube using a syringe. After stirring for 60 min, 1 mL of 5% HCl-methanol solution was added to quench the reaction.

The quenched reaction solution was added dropwise to 20 mL of ice methanol, the supernatant was discarded after centrifugation, and the precipitate was polyHL. The above purification steps were repeated 3-5 times, and the precipitate collected in the last time was dried in a vacuum drying oven until its weight was constant to obtain the polyHL_160 sample. The corresponding GPC raw data were shown in FIG. 56, and Mₙ and Ð were measured.

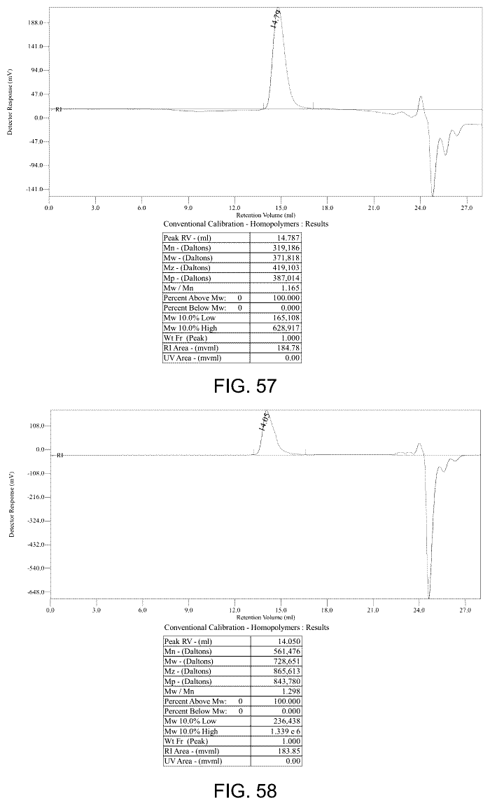

Preparation of polyHL_319 (molecular weight Mₙ is 319) cyclic polymer sample: the preparation procedures were almost the same as those in Example polyHL_160, except that the equivalent amount of catalyst was changed to 0.013 mmol and the reaction time was changed to 4 h. The corresponding GPC raw data were shown in FIG. 57, and Mₙ and Ð were measured.

Preparation of polyHL_562 (molecular weight Mₙ is 562) cyclic polymer sample: the preparation procedures were almost the same as those in Example polyHL_160, except that the equivalent amount of catalyst was changed to 0.0063 mmol and the reaction time was changed to 6 h. The corresponding GPC raw data were shown in FIG. 57, and Mₙ and Ð were measured.

### (2) Determination of Polymer Peel Properties

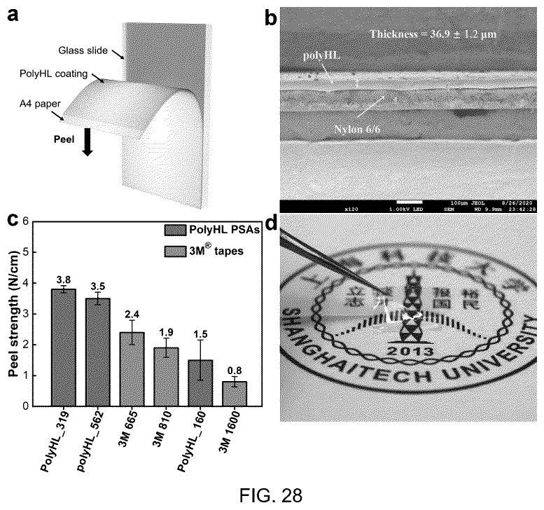

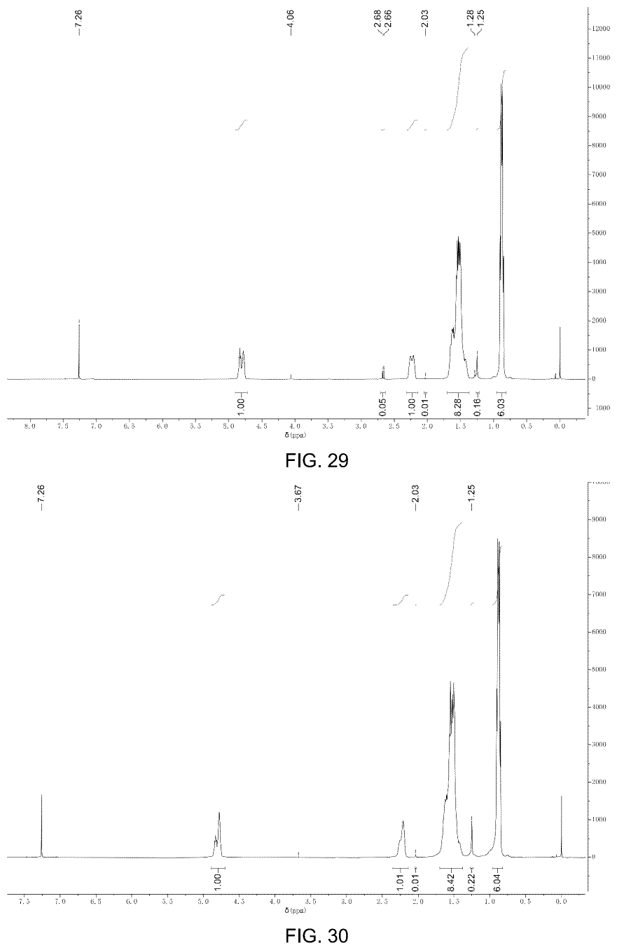

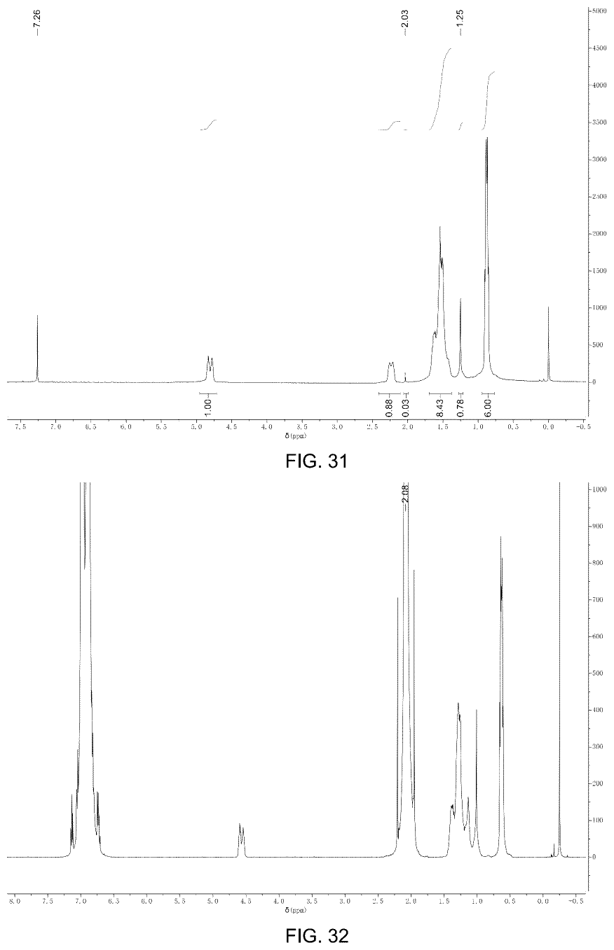

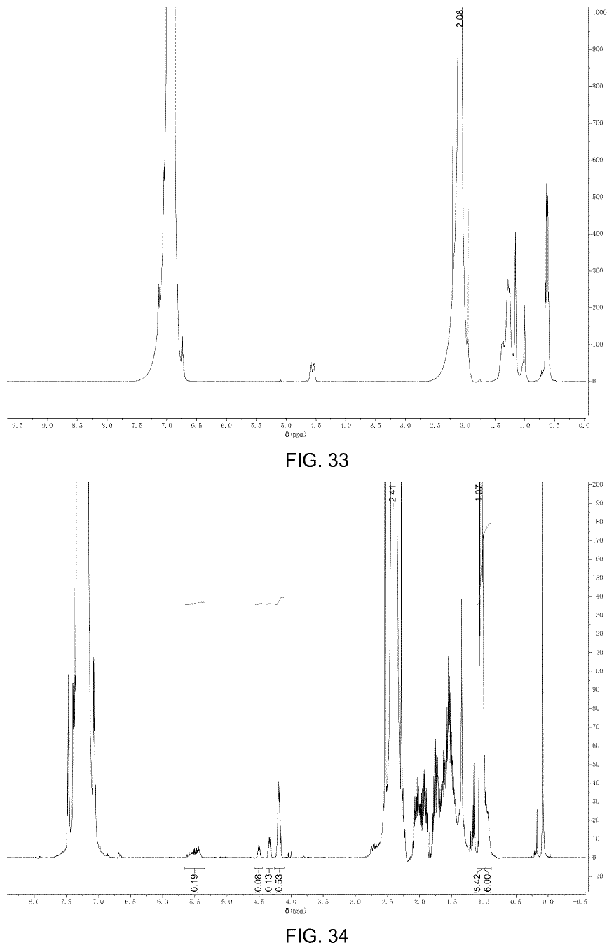

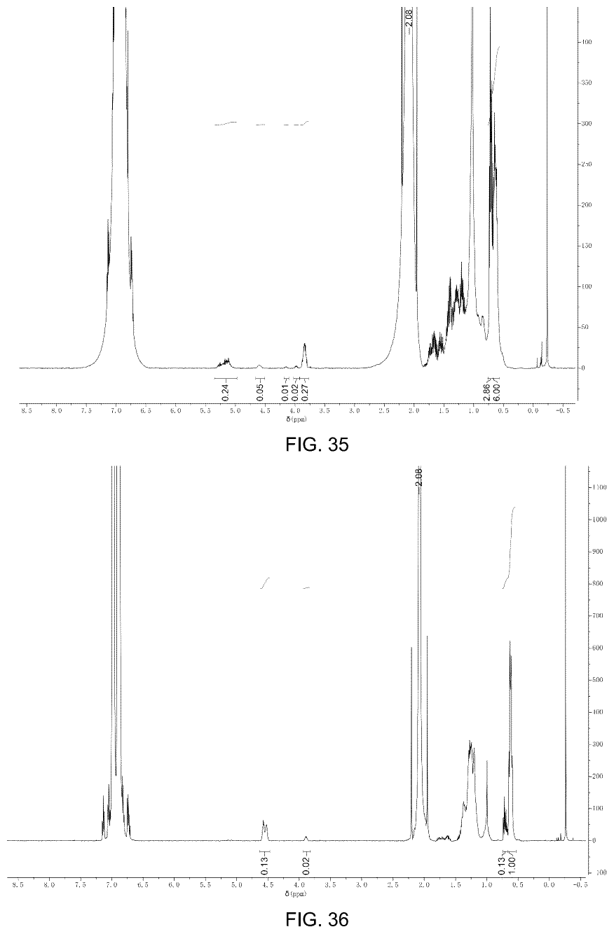

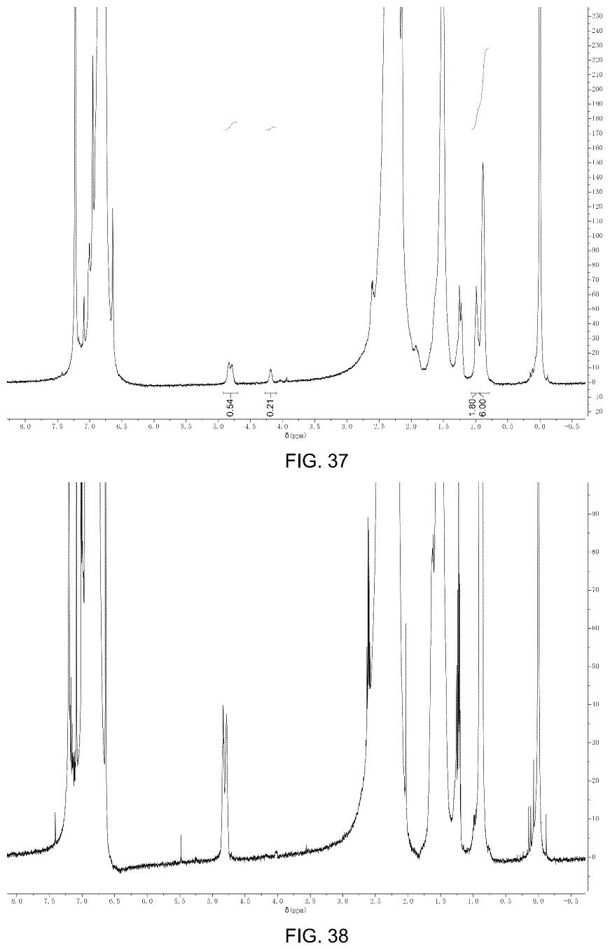

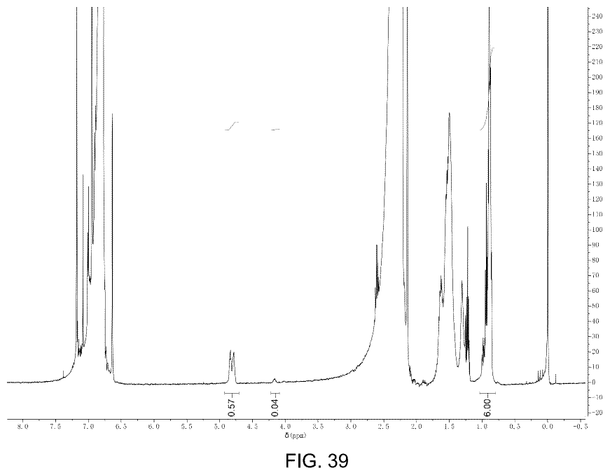

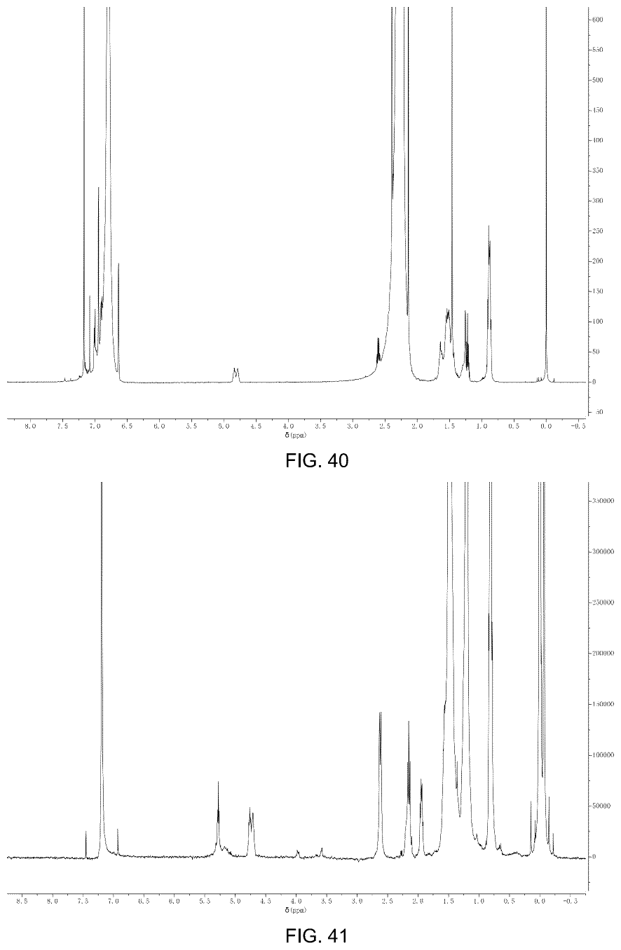

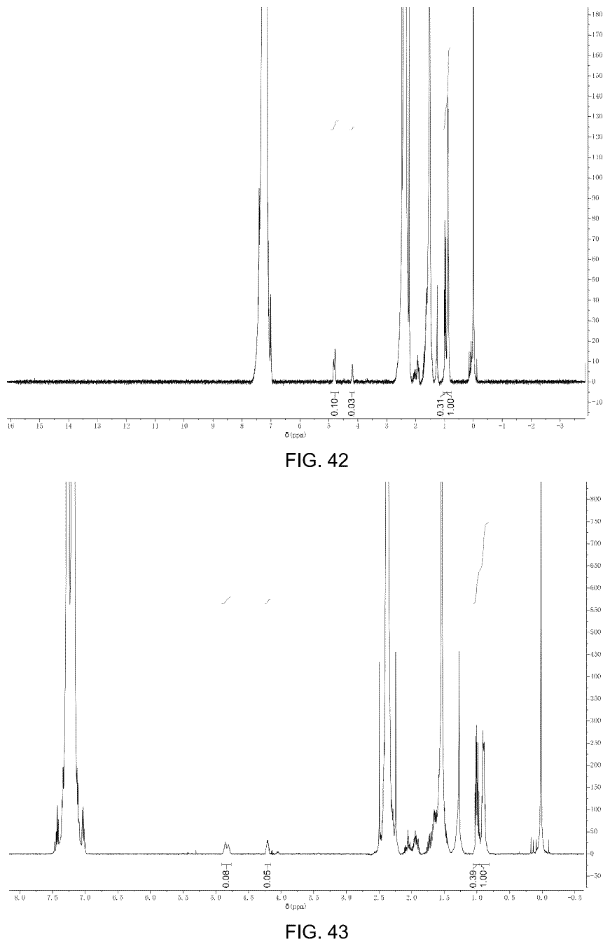

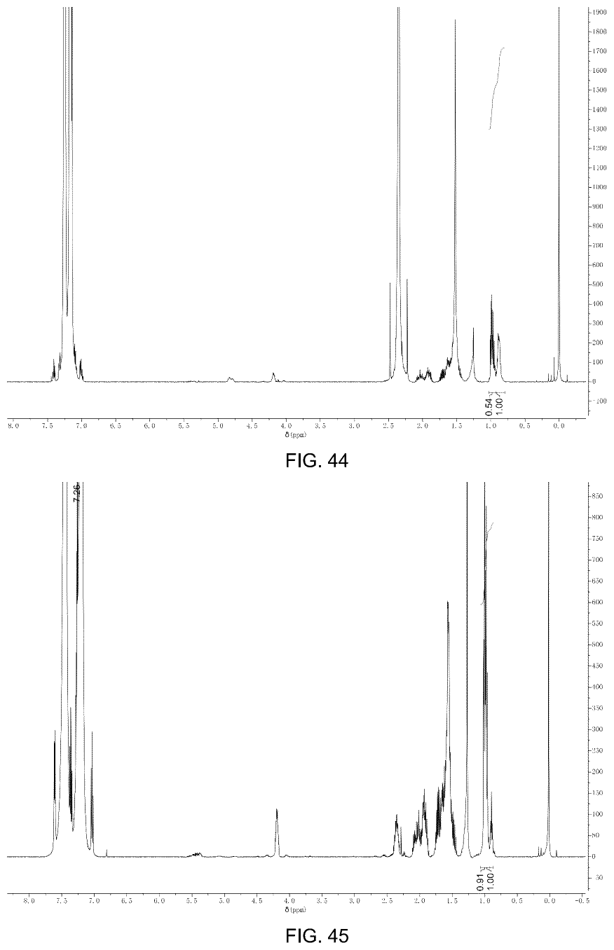

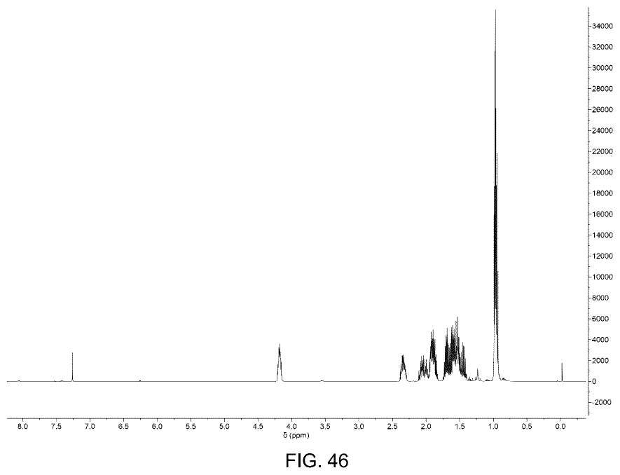

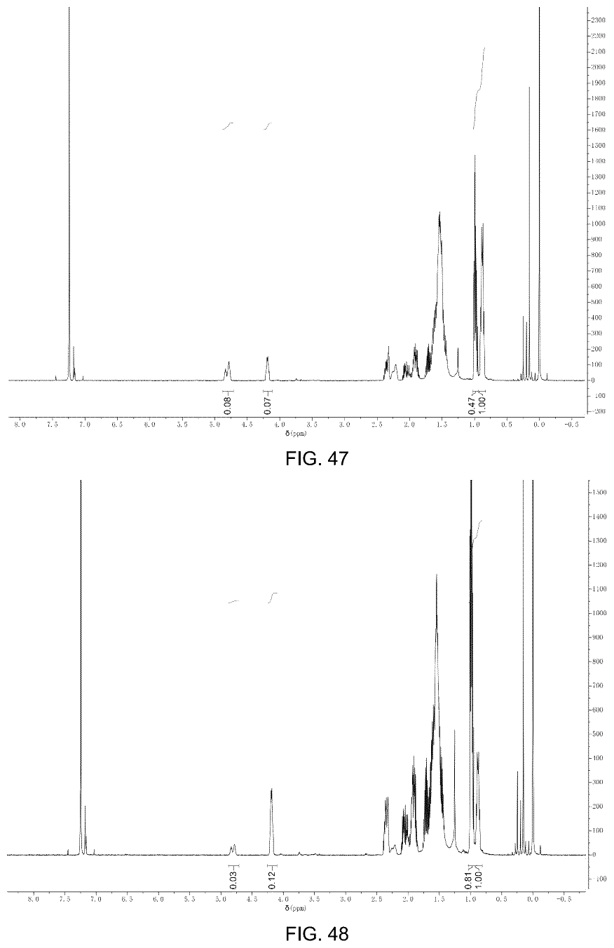

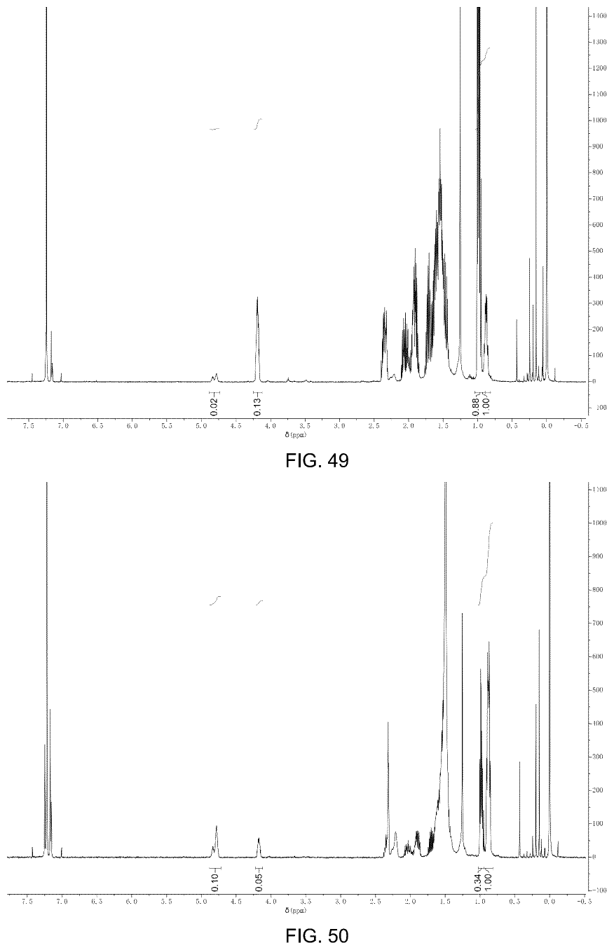

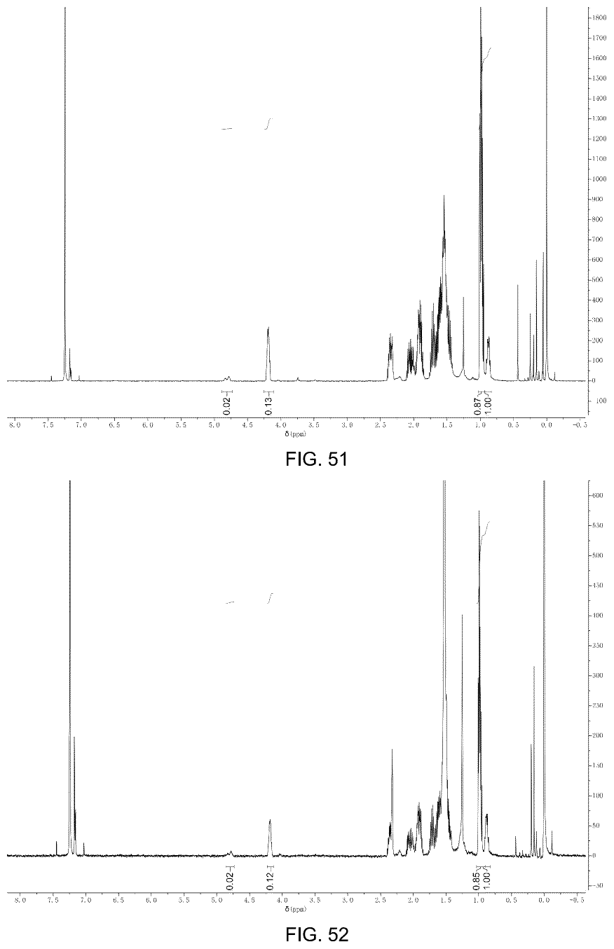

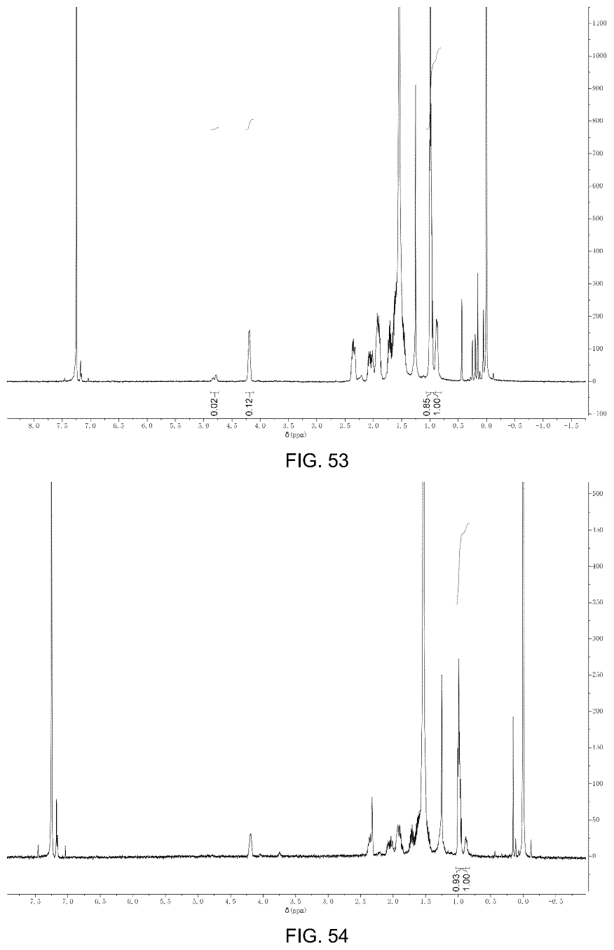

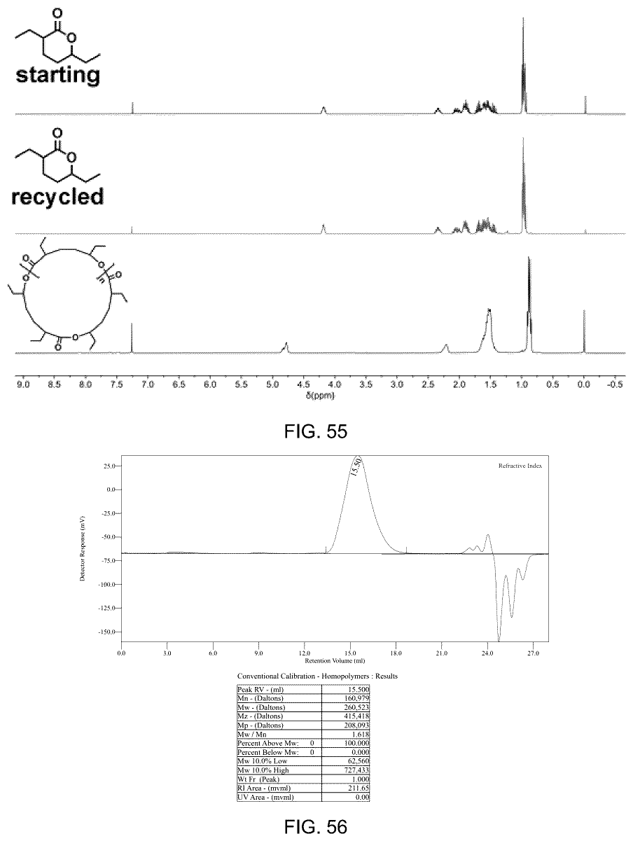

A slide was used as the hard substrate, A4 paper (15×2.6 cm) was used as the surface substrate (FIG. 28a), and the polyHL samples were uniformly coated on the slide with a coating blade (3M 665, 3M810, and 3M1600 were used as controls). Cross-sectional scanning electron microscopy (SEM) showed a uniform film thickness of 36.9.±1.2 μm (FIG. 28b). The experiments were conducted at 25° C. on an Instron 5966 universal mechanical tester, with a peel angle of 180° and a peel speed of 10 mm min-1. It is noteworthy that the adhesion strengths of polyHL_319, 562, and 160 (where the numbers refer to the number average molecular weight Mₙ of polyHL, which are 319, 562, and 160 kg mol⁻¹, respectively) were 3.8±0.12, 3.5±0.20, and 1.5±0.65 N cm⁻¹, respectively (FIG. 28c and Table 5). Under the same test conditions, the peel strengths of polyHL_319 and polyHL_562 were higher than those of the 3M®Scotch commercial transparent tapes (3M®665 was 2.4-0.40 N cm^{-1; 3}M®810 was 1.9±0.31 N cm⁻¹) and the vinyl electrical tape (3M®1600 was 0.8±0.17 N cm⁻¹). The peel strength of PolyHL_160 was 1.5±0.65 N cm⁻¹, equivalent to that of 3M®Scotch commercial transparent tape 3M®810.

### (3) Determination of Light Transmission, Flexibility, and Viscoelasticity

To demonstrate the light transmission and color of polyHL, high molecular weight polymer samples (Mₙ=613.8 kg mol⁻¹, Ð=1.45, Embodiment 29) were made into transparent, colorless polymer films on PTFE molds using a solution-casting method. These films exhibited good flexibility and viscoelasticity (FIG. 28d).

TABLE 5
Measured data of 180° peel properties of cyclic polyHL samples and
three commercialized tapes (the same sample was repeatedly tested three times)

| | polyHL_562 | polyHL319 | polyHL_160 | 3M ® 665 | 3M ® 810 | 3M ® 1600 |
|---|---|---|---|---|---|---|
| Peel Strength_1b | 3.280 | 3.926 | 1.217 | 2.544 | 1.547 | 0.6220 |
| (N/cm) | | | | | | |
| Peel Strength_2b | 3.667 | 3.667 | 2.241 | 2.746 | 1.853 | 0.8250 |
| (N/cm) | | | | | | |
| Peel Strength_3b | 3.586 | 3.756 | 1.025 | 1.976 | 2.175 | 0.9572 |
| (N/cm) | | | | | | |
| Average peel | 3.51 | 3.79 | 1.49 | 2.42 | 1.86 | 0.80 |
| strength (N/cm) | | | | | | |
| Standard | 0.20 | 0.12 | 0.65 | 0.40 | 0.31 | 0.17 |
| deviation | | | | | | |

### Embodiments 50-64: Chemical Recycling Experiments of Cyclic polyHL Polymers at High Temperatures

The chemical recyclability of the polyHL sample prepared in Embodiment 30 was determined, where the sample had an Mₙ of 500-600 kg mol⁻¹, which was specifically 571.5 kg mol⁻¹.

Firstly, several trifluoromethanesulfonic acid metal salts, including AgCF₃SO₃, Cu(CF₃SO₃)₂, Fe(CF₃SO₃)₃, Sc(CF₃SO₃)₃, and Y(CF₃SO₃)₃, were used to catalyze the polyHL (toluene solution, initial concentration of HL [M]₀=0.5 M) in a closed reaction tube at 120° C. for 24 h (Embodiments 50-54). Only Fe(CF₃SO₃)₃ and Sc(CF₃SO₃)₃ could degrade the polyHL sample to recycle the HL monomer, and the recycling rates were 53% and 27%, respectively (Embodiments 52, 53).

Then, on the basis of Embodiment 50, the catalyst, solvent, and reaction temperature of the recycling experiment were changed, and the experimental parameters were specified in Table 6. The results showed that only FeCl₂ could degrade the polyHL sample to recycle the HL monomer with a recycling rate of 21% (Embodiments 55); Sn(Oct)₂ had a recycling rate of only 5%; and Fe(acac)₂, DBTDL, and ^{t}Bu-P₄ had no significant reactivity even at 150° C. for 12 h (Embodiments 55-59).

Later, the reactions were carried out in toluene with ZnCl₂ as catalyst for 12 h at 130° C., 140° C. and 150° C., respectively. The recycling rate gradually increased with increasing temperature (Embodiments 60-62; 31%, 39%, and 54%). Interestingly, when using the higher polarity solvent ortho-dichlorobenzene at 150° C. and 160° C., the recycling rates significantly increased to 91% and 100%, respectively (Embodiments 63-64), indicating the ZnCl₂ catalyst can realize a 100% chemical recycling.

TABLE 6
Chemical recycling experiments of cyclic polyHL samples

| | | | | | HL Recycling |
|---|---|---|---|---|---|
| | | Temperature | Time | | rate |
| Embodiment | Catalyst | (° C.) | (h) | Solvent | (%) |
| 24 | AgCF₃SO₃ | 120 | 24 | Toluene | — |
| 25 | Cu(CF₃SO₃)₂ | 120 | 24 | Toluene | — |
| 26 | Fe(CF₃SO₃)₃ | 120 | 24 | Toluene | 53 |
| 27 | Sc(CF₃SO₃)₃ | 120 | 24 | Toluene | 27 |
| 28 | Y(CF₃SO₃)₃ | 120 | 24 | Toluene | — |
| 29 | FeCl₂ | 150 | 12 | Homotrimethylbenzene | 21 |
| 30 | Fe(acac)₂ | 150 | 12 | Trimethylbenzene | — |
| 31 | Sn(Oct)₂ | 150 | 12 | Homotrimethylbenzene | 5 |
| 32 | DBTDL | 150 | 12 | Homotrimethylbenzene | — |
| 33 | ^{t}Bu—P₄ | 150 | 12 | Homotrimethylbenzene | — |
| 34 | ZnCl₂ | 130 | 12 | Toluene | 31 |
| 35 | ZnCl₂ | 140 | 12 | Toluene | 39 |
| 36 | ZnCl₂ | 150 | 12 | Toluene | 54 |
| 37 | ZnCl₂ | 150 | 12 | o-Dichlorobenzene | 91 |
| 38 | ZnCl₂ | 160 | 12 | o-Dichlorobenzene | 100 |

### Embodiment 50

In a glove box under nitrogen atmosphere, 200 mg of cyclic polyHL sample (prepared in Embodiment 40) and 5 mol % of AgCF₃SO₃ were added to a 25 mL Schlenk tube, and then 2.6 mL of toluene was added to for their dissolution. The Schlenk tube was then sealed with a rubber stopper, wrapped with a sealing film, taken out of the glove box, and placed in a pre-set 120° C. oil bath. After stirring for 24 h, the solvent was removed from the system, and 10 mg of degraded product was subjected to ¹H NMR for HL yield.

### Embodiments 51-54

The experimental conditions were almost the same as those in Embodiment 50, except that AgCF₃SO₃ was replaced with Cu(CF₃SO₃)₂ (Embodiment 51), Fe(CF₃SO₃)₃ (Embodiment 52), Sc(CF₃SO₃)₃ (Embodiment 53), Y(CF₃SO₃)₃ (Embodiment 54).

### Embodiments 55-59

The experimental conditions were almost the same as those in Embodiment 50, except that the catalyst was changed, the reaction temperature was raised to 150° C., the reaction time was shortened to 12 h, and the solvent was replaced with mesitylene. The catalysts included FeCl₂ (Embodiment 55), Fe(acac) 2 (Embodiment 56), Sn(Oct)₂ (Embodiment 57), DBTDL (Embodiment 58), ^{t}Bu-P₄ (Embodiment 59).

### Embodiment 60

In a glove box under nitrogen atmosphere, 200 mg of a cyclic polyHL sample (prepared in Embodiment 40) and 5 mol % ZnCl₂ were added to a 25 mL Schlenk tube, and then 2.6 mL of toluene was added for their dissolution. The Schlenk tube was then sealed with a rubber stopper, wrapped with a sealing film, taken out of the glove box, and placed in a pre-set 130° C. oil bath. After stirring for 12 h, the solvent was removed from the system, and 10 mg of degraded product was subjected to ¹H NMR for HL yield.

### Embodiments 61-62

The experimental conditions were almost the same as those in Embodiment 60, except that the reaction temperature was raised to 140° C. (Embodiment 61) and 150° C. (Embodiment 62).

### Embodiments 63-64

The experimental conditions were almost the same as those in Embodiment 60, except that the reaction temperature and solvent were changed: 150° C., ortho-dichlorobenzene (Embodiment 63); 160° C., ortho-dichlorobenzene (Embodiment 64).

### Embodiments 65-73: Chemical Recycling Experiments of Cyclic polyHL Polymers Under Mild Conditions

To further reduce the energy input during the chemical recycling process, the catalytic activity of IMes, DBU, TBD, and La(La[N(SiMe₃)₂]₃) was investigated in the present disclosure. At 50° C., (toluene as solvent, [HL]₀=0.5 M), only La[N(SiMe₃)₂]₃ exhibited a good HL yield, where the yield was 47% at 3 h, 81% at 12 h, and 88% at 24 h (Embodiments 65-67).

When [HL]₀ in the above system was adjusted to 0.1 M, no significant increase in HL yield was observed (Embodiments 68-69). To achieve complete recycling of the HL monomer, the reaction temperature was raised to 80° C. with the toluene as solvent and [HL]₀=0.5 M in the present disclosure. The yield of the HL monomer reached 85% at 3 h and remained constant within 12 h (Embodiments 70-71), indicating that the depolymerization at 80° C. is more rapid than that at 50° C. The system was then diluted to 0.1 M at 80° C., and the HL monomer yield reached 93% at 3 h (Embodiment 72), and finally 100% at 12 h (Embodiment 73). The results were shown in Table 7.

TABLE 7
Degradation experiments of cyclic polyHL under mild conditions

| | | Tem- | | | | HL |
|---|---|---|---|---|---|---|
| Em- | | perature | [HL]₀ | Time | | recycling |
| bodiment | Catalyst | (° C.) | (mol/L) | (h) | Solvent | rate (%) |
| 39 | La[N(SiMe₃)₂]₃ | 50 | 0.5 | 3 | Toluene | 47 |
| 40 | La[N(SiMe₃)₂]₃ | 50 | 0.5 | 12 | Toluene | 81 |
| 41 | La[N(SiMe₃)₂]₃ | 50 | 0.5 | 24 | Toluene | 88 |
| 42 | La[N(SiMe₃)₂]₃ | 50 | 0.1 | 3 | Toluene | 34 |
| 43 | La[N(SiMe₃)₂]₃ | 50 | 0.1 | 24 | Toluene | 87 |
| 44 | La[N(SiMe₃)₂]₃ | 80 | 0.5 | 3 | Toluene | 85 |
| 45 | La[N(SiMe₃)₂]₃ | 80 | 0.5 | 12 | Toluene | 85 |
| 46 | La[N(SiMe₃)₂]₃ | 80 | 0.1 | 3 | Toluene | 93 |
| 47 | La[N(SiMe₃)₂]₃ | 80 | 0.1 | 12 | Toluene | 100 |

### Embodiment 65

In a glove box under nitrogen atmosphere, 200 mg of a cyclic polyHL sample (prepared in Embodiment 40) and 3 mol % (La[N(SiMe₃)₂]₃) were added to a 25 mL Schlenk tube, and then 2.6 mL of toluene was added for their dissolution. The Schlenk tube was then sealed with a rubber stopper, wrapped with a sealing film, taken out of the glove box, and placed in a pre-set 50° C. oil bath. After stirring for 3 h, the solvent was removed from the system, and 10 mg of degraded product was subjected to ¹H NMR for HL yield.

### Embodiments 66-69

The experimental conditions were almost the same as those in Embodiment 65, except that the amount of reaction solvent and/or the reaction time were varied: 2.6 mL toluene, 12 h (Embodiment 66); 2.6 mL toluene, 24 h (Embodiment 67); 12.8 mL toluene, 3 h (Embodiment); 12.8 mL toluene, 24 h (Embodiment 69).

### Embodiment 70

In a glove box under nitrogen atmosphere, 200 mg of a cyclic polyHL sample (prepared in Embodiment 40) and 3 mol % (La[N(SiMe₃)₂]₃) were added to a 25 mL Schlenk tube, and then 2.6 mL of toluene was added for their dissolution. The Schlenk tube was then sealed with a rubber stopper, wrapped with a sealing film, taken out of the glove box, and placed in a pre-set 80° C. oil bath. After stirring for 3 h, the solvent was removed from the system, and 10 mg of degraded product was subjected to ¹H NMR for HL yield.

### Embodiments 71-73

The experimental conditions were almost the same as those in Embodiment 70, except that the amount of reaction solvent and/or the reaction time were varied: 2.6 mL toluene, 12 h (Embodiment 71); 12.8 mL toluene, 3 h (Embodiment 72); 12.8 mL toluene, 12 h (Embodiment 73).

To obtain the target monomer δLH₂, δ-L was used as raw material, Stryker reagent ([(Ph₃P)CuH]₆) was used as the catalyst, and triethoxysilane (His(OEt)₃) was used as the hydrogen source. After stirring for 6 h in a toluene solution at ambient temperature, the preparation of δLH₂ monomer was successfully realized in the present disclosure for the first time.

### Embodiment 74: Preparation of δLH₂ monomer

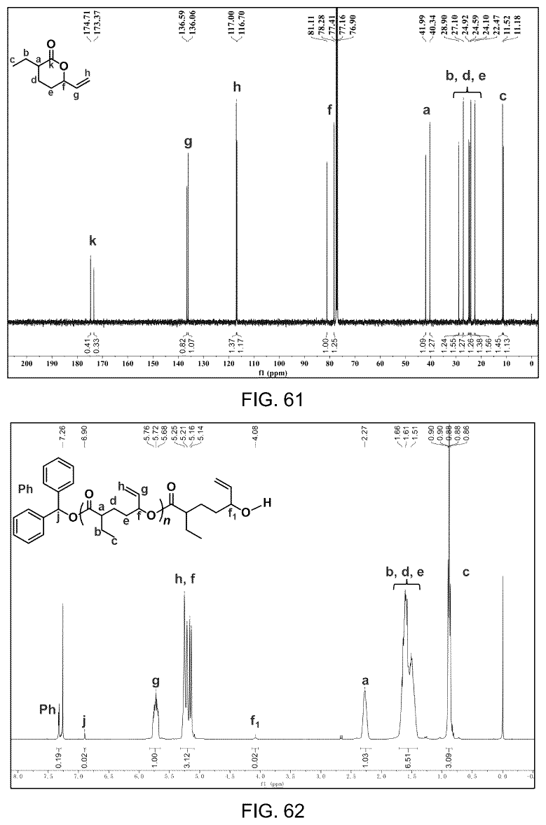

In a glove box under nitrogen atmosphere, 109.7 mg (0.17 mol %) of Stryker reagent and 11.9 g (2.2 equiv) of triethoxysilane were added to an eggplant-shaped flask, and 250 ml of toluene was added for their dissolution. After stirring for 0.5 h, about 5.0 g of δ-L was added dropwise to the above flask, and the reaction was conducted for 6 h at room temperature after the addition of δ-L. After the reaction, the flask was taken out from the glove box, added with a large amount of saturated sodium bicarbonate solution to quench the reaction, and extracted three times with chloroform and sodium bicarbonate solution. The obtained organic layers were combined and dried using anhydrous sodium sulfate, filtered, and evaporated to remove the solvent to obtain the crude product. The crude product was then subjected to column chromatography (petroleum ether/ethyl acetate=8/1), and the resulting pure product was a colorless oily liquid. The corresponding NMR hydrogen spectrum and NMR carbon spectrum were shown in FIGS. 60-61. The yield of the prepared δLH₂ monomer was 83% in the present disclosure, the diastereoisomer ratio of the resulting product was 55/45, and the product was a colorless oily liquid.

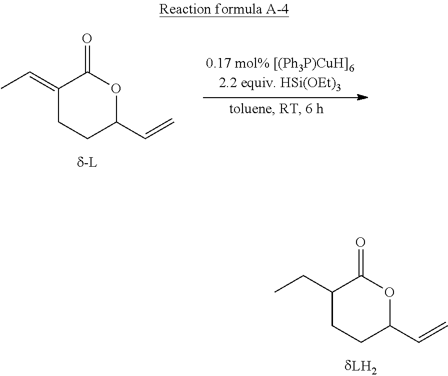

### Embodiments 75˜81: ^{t}Bu-P₄-Catalyzed Ring-Opening Polymerization of δLH₂

^{t}Bu-P₄ polyphosphazene was employed as a catalyst for the anionic ring-opening polymerization of δLH₂ in the present disclosure. With a feeding ratio [δLH₂]/[^{t}Bu-P₄]=500/1 (molar ratio), the system was stirred in a THF solution at −25° C. for 24 h ([δLH₂]₀=5.82 M), the conversion rate of δLH₂ was 84%, and the resulting polymer, poly(δLH₂), had a high molecular weight of Mₙ=246.0 kg mol⁻¹ and a wide molecular weight distribution Ð=2.40 (Embodiment 75). If phenyl methanol (BnOH) was used as an additional alcohol initiator, the polymerization reaction is carried out with a feeding ratio of [δLH₂]/[BnOH]=50/1 (molar ratio). As the equivalent amount of polyphosphazene catalyst decreased (from 2, 1, 0.4 to 0.2 mol %), the molecular weight and molecular weight distribution of the resulting polymer tended to decrease, i.e., the molecular weight tended to approach the calculated molecular weight from the feeding ratio (Embodiments 76˜79). If the phenyl methanol initiator was replaced with diphenylmethanol, a similar trend in molecular weight and molecular weight distribution can also be observed (Embodiments 80˜81, where the NMH spectrum of poly(δLH₂) prepared in Embodiment 81 was shown in FIG. 62).

### Embodiment 75

In a glove box under nitrogen atmosphere, 0.00134 mmol of ^{t}Bu-P₄ catalyst was added to a flame-dried 10 mL Schlenk tube, and then 0.016 mL of THF was added to dissolve the ^{t}Bu-P₄. The Schlenk tube was then sealed with a rubber stopper, wrapped with a sealing film, taken out of the glove box, and placed in a pre-set −25° C. low temperature bath until the temperature stabilized. 0.67 mmol of δLH₂ was rapidly injected into the Schlenk tube using a syringe. After stirring for 24 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was poly(δLH₂). The above purification steps were repeated 3-5 times, and the precipitate collected in the last time was dried in a vacuum drying oven until its weight was constant to obtain the purified poly(δLH₂) sample. Mₙ (number average molecular weight) and Ð (molecular weight distribution) were measured by the GPC method at 40° C., in which tetrahydrofuran is the mobile phase and PMMA is the standard sample for calibration.

### Embodiment 76

In a glove box under nitrogen atmosphere, 0.0134 mmol of ^{t}Bu-P₄ catalyst and 0.0134 mmol of BnOH initiator were added to a flame-dried 10 mL Schlenk tube, and then 0.016 mL of THF was added for their dissolution. The Schlenk tube was then sealed with a rubber stopper, wrapped with a sealing film, taken out of the glove box, and placed in a pre-set −25° C. low temperature bath until the temperature stabilized. 0.67 mmol of δLH₂ was rapidly injected into the Schlenk tube using a syringe. After stirring for 12 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was poly(δLH₂). The above purification steps were repeated 3-5 times, and the precipitate collected in the last time was dried in a vacuum drying oven until its weight was constant to obtain the purified poly(δLH₂) sample. Mₙ (number average molecular weight) and Ð (molecular weight distribution) were measured by the GPC method at 40° C., in which tetrahydrofuran is the mobile phase and PMMA is the standard sample for calibration.

### Embodiments 77˜79

The preparation procedures were almost the same as those in Embodiment 76, except that the equivalent amount is changed: Embodiment 77 (0.0067 mmol), Embodiment 78 (0.00268 mmol), Embodiment 79 (0.00134 mmol).

### Embodiment 80

The preparation procedures were almost the same as those in Embodiment 76, except that the BnOH initiator in Embodiment 76 was replaced with a diphenylmethanol (Ph₂CHOH) initiator.

### Embodiment 81

The preparation procedures were almost the same as those in Embodiment 79, except that the BnOH initiator in Embodiment 79 was replaced with a diphenylmethanol (Ph₂CHOH) initiator.

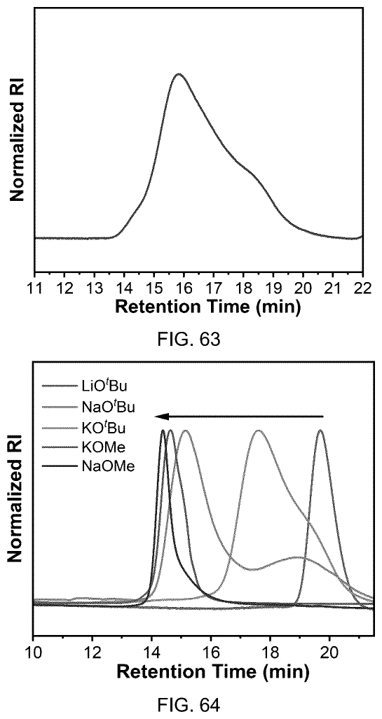

Encouraged by the preliminary results of ring-opening polymerization (ROP), the present disclosure further investigated the feasibility of using several alkali metal alkoxides as ROP initiators. Initially, in THE solution, the present disclosure achieved an 86% conversion of δLH₂ using KOMe (potassium methoxide) and BnOH as co-initiators and a feeding ratio of [δLH₂]/[KOMe]/[BnOH]=50/0.5/1 (molar ratio). The produced poly(δLH₂) had a molecular weight of Mₙ=73.4 kg mol⁻¹ and a molecular weight distribution of Ð=2.27 (Embodiment 82). It is inferred that this relatively uncontrollable polymerization may be caused by the competitive dual initiation of KOMe and BnOK after the addition of BnOH (FIG. 63). Later, only KOMe was used as the initiator, and the reaction was conducted in THF at −25° C. for 24 h at a feeding ratio of [δLH₂]/[KOMe]=50/1. The conversion reached 94%, and the resulting poly(δLH₂) had a very high molecular weight of Mₙ=442.8 kg mol⁻¹ and a rather narrow molecular weight distribution of Ð=1.20 (Embodiment 83).

### Embodiments 82˜85: Ring-Opening Polymerization of δLH₂ Initiated by Methanol

### Embodiment 82

In a glove box under nitrogen atmosphere, 0.0067 mmol of KOMe initiator and 0.0134 mmol of BnOH initiator were added to a flame-dried 10 mL Schlenk tube, and then 0.02 mL of THF was added for their dissolution. The Schlenk tube was then sealed with a rubber stopper, wrapped with a sealing film, taken out of the glove box, and placed in a pre-set −25° C. low temperature bath until the temperature stabilized. 0.67 mmol of δLH₂ was rapidly injected into the Schlenk tube using a syringe. After stirring for 24 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was poly(δLH₂). The above purification steps were repeated 3-5 times, and the precipitate collected in the last time was dried in a vacuum drying oven until its weight was constant to obtain the purified poly(δLH₂) sample. Mₙ (number average molecular weight) and Ð (molecular weight distribution) were measured by the GPC method at 40° C., in which tetrahydrofuran is the mobile phase and PMMA is the standard sample for calibration.

### Embodiment 83

In a glove box under nitrogen atmosphere, 0.0134 mmol of KOMe initiator was added to a flame-dried 10 mL Schlenk tube, and then 0.02 mL of THF was added to dissolve KOMe. The Schlenk tube was then sealed with a rubber stopper, wrapped with a sealing film, taken out of the glove box, and placed in a pre-set −25° C. low temperature bath until the temperature stabilized. 0.67 mmol of δLH₂ was rapidly injected into the Schlenk tube using a syringe. After stirring for 24 h, 1 mL of 5% HCl-methanol solution was added to quench the reaction. 50 μL of the reaction solution was taken out for ¹H NMR analysis to determine the monomer conversion rate. The quenched reaction solution was added dropwise to 20 mL of ice methanol, the supernatant was discarded after centrifugation, and the precipitate was poly(δLH₂). The above purification steps were repeated 3-5 times, and the precipitate collected in the last time was dried in a vacuum drying oven until its weight was constant to obtain the purified poly(δLH₂) sample. Mₙ (number average molecular weight) and Ð (molecular weight distribution) were measured by the GPC method at 40° C., in which tetrahydrofuran is the mobile phase and PMMA is the standard sample for calibration.

### Embodiment 84

The preparation procedures were almost the same as those in Embodiment 83, except that the equivalent amount of KOMe initiator was reduced to 0.0067 mmol.

### Embodiment 85

The preparation procedures were almost the same as those in Embodiment 83, except that the reaction time was extended to 48 h and the equivalent amount of KOMe initiator was reduced to 0.00335 mmol.

TABLE 8
Ring-opening polymerization results of
δLH₂ under different catalysis systems

| | | | δLH₂/ | | | Con- | Mₙ | |
|---|---|---|---|---|---|---|---|---|
| Em- | | | Catalyst/ | [δLH₂]₀ | Time | version | (kg | |
| bodiment | Catalyst | Initiator | Initiator | (M) | (h) | rate (%) | mol⁻¹) | Ð |
| 2 | ^{t}Bu-P₄ | | 50/0.1/0 | 5.82 | 24 | 84 | 246.0 | 2.40 |
| 3 | ^{t}Bu-P₄ | BnOH | 50/1/1 | 5.82 | 12 | 82 | 15.3 | 1.55 |
| 4 | ^{t}Bu-P₄ | BnOH | 50/0.5/1 | 5.82 | 12 | 87 | 14.4 | 1.63 |
| 5 | ^{t}Bu-P₄ | BnOH | 50/0.2/1 | 5.82 | 12 | 89 | 14.3 | 1.36 |
| 6 | ^{t}Bu-P₄ | BnOH | 50/0.1/1 | 5.82 | 12 | 92 | 11.5 | 1.16 |
| 7 | ^{t}Bu-P₄ | Ph₂CHOH | 50/1/1 | 5.82 | 12 | 84 | 17.9 | 1.82 |
| 8 | ^{t}Bu-P₄ | Ph₂CHOH | 50/0.1/1 | 5.82 | 12 | 92 | 11.7 | 1.10 |
| 9 | | KOMe/BnOH | 50/0/(0.5:1)d | 5.62 | 24 | 86 | 73.4 | 2.27 |
| 10 | | KOMe | 50/0/1 | 5.62 | 24 | 94 | 442.8 | 1.20 |
| 11 | | KOMe | 100/0/1 | 5.62 | 24 | 95 | 579.6 | 1.20 |
| 12 | | KOMe | 200/0/1 | 5.62 | 48 | 81 | 587.7 | 1.14 |

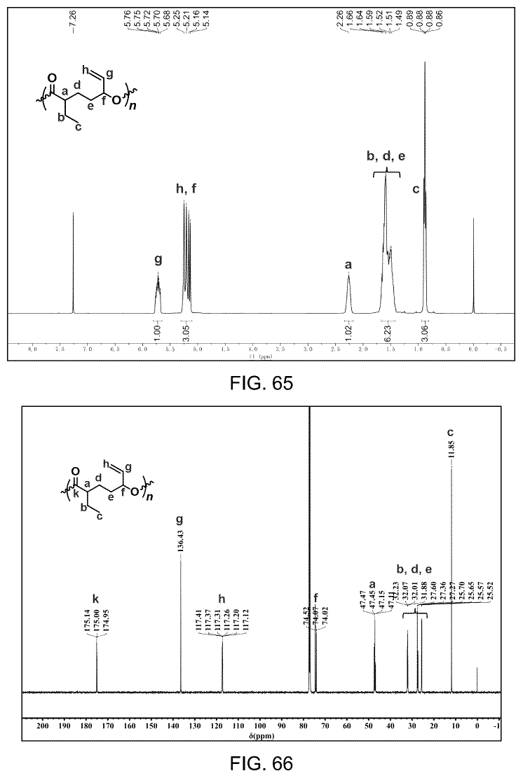

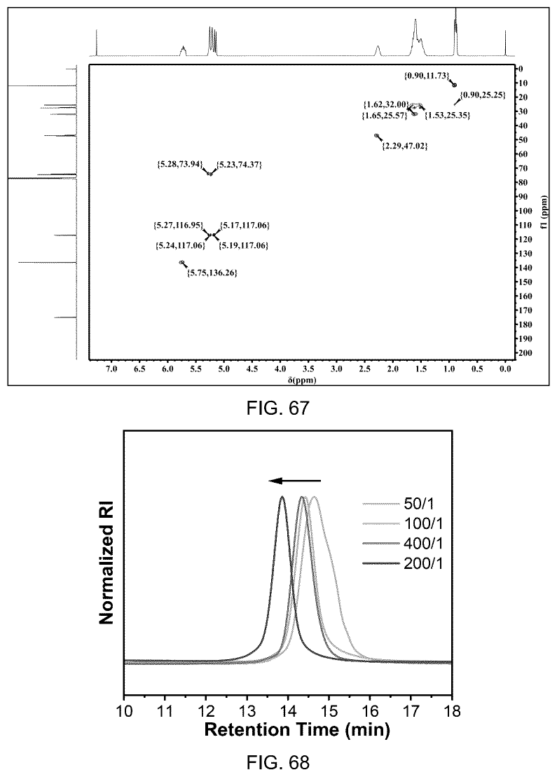

Under the same polymerization conditions, several other common alkali metal alkoxides, including NaOMe, KO^{t}Bu, NaO^{t}Bu, and LiO^{t}Bu (FIG. 64) were investigated in the present disclosure. As shown in Tables 8 and 9, the produced poly(δLH₂) initiated by KOMe and NaOMe had high molecular weights and desirable controllability (Embodiment 83 and FIGS. 65˜67, Mₙ=442.8 kg mol⁻¹, Ð=1.20; Embodiment 86, Mₙ=534.6 kg mol⁻¹,
=1.17). As for KO^{t}Bu and NaO^{t}Bu, the corresponding GPC curves exhibited bimodal distributions, likely due to the presence of rapid and uncontrollable ester exchange reactions in these systems (Embodiments 87˜88). The obtained Poly(δLH₂) initiated by LiO^{t}Bu had a significantly low molecular weight and a narrow molecular weight distribution, at the same time, the monomer conversion rate was reduced, suggesting that LiO^{t}Bu is an initiator with low reactivity but good controllability (Embodiment 89). In addition, in THF at −25° C., the Mₙ of the resulting polymers increased slightly while the molecular weight distribution remained narrow as the feeding equivalent amount of KOMe decreased from 2 mol % to 1.0 mol % and 0.5 mol %, respectively (FIG. 68, Embodiments 83˜85). Poly(δLH₂) with very high molecular weight and very narrow molecular weight distribution was generated when using 0.5 mol % KOMe (Embodiment 85, Mₙ=587.7 kg mol⁻¹, Ð=1.14).

### Embodiments 86˜89: Ring-Opening Polymerization of δLH₂ Involving different alkali metal alkoxide initiators

The preparation procedures were almost the same as those in Embodiment 83, except that KOMe was replaced: Embodiment 86 (sodium methoxide, NaOMe), Embodiment 87 (potassium tert-butoxide, KO^{t}Bu), Embodiment 88 (sodium tert-butoxide, NaO^{t}Bu), Embodiment 89 (lithium tert-butoxide, LiO^{t}Bu).

TABLE 9
Ring-opening polymerization results of
δLH₂ under different initiation systems

| | | | Conversion | | |
|---|---|---|---|---|---|
| | | δLH₂/ | rate | Mₙ | |
| Embodiment | Initiator | initiator | (%) | (kg mol⁻¹) | Ð |
| 13 | NaOMe | 50/1 | 92 | 534.6 | 1.17 |
| 14 | KOtBu | 50/1 | 86 | 213.6 | 1.42 |
| 15 | NaOtBu | 50/1 | 86 | 31.3 | 1.48 |
| 16 | LiOtBu | 50/1 | 48 | 12.7 | 1.09 |

### Embodiment 90: Thermodynamic Properties

The thermodynamic stability of poly(δLH₂) prepared under different conditions can be analyzed by thermogravimetric analysis (TGA) and differential scanning calorimeter (DSC).

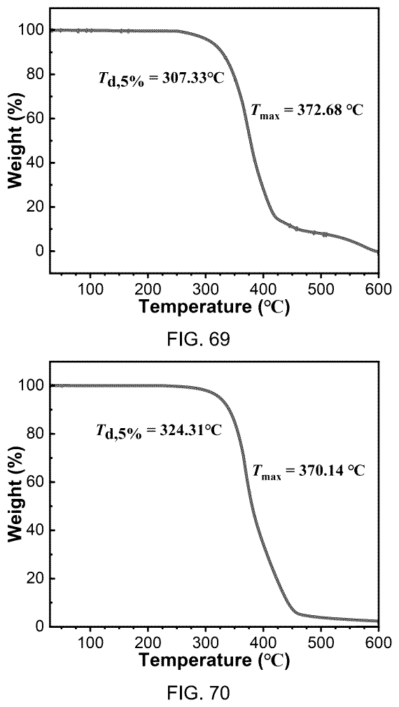

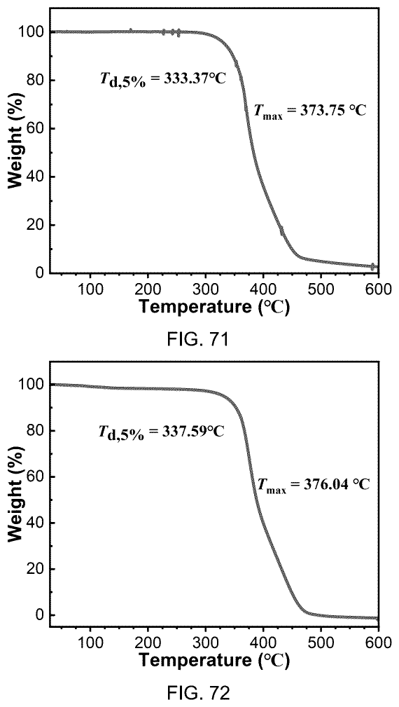

Poly(δLH₂) prepared using ^{t}Bu-P₄ as the catalyst and BnOH as the initiator (Embodiment 79), and poly(δLH₂) prepared without catalyst and using only KOMe (Embodiments 82-84) as initiator both have good thermal stability (5% weight loss temperatures were both greater than 300° C.). The TGA curves revealed that the T_{d,5%} and Tₘₐₓ of the different samples were related to their molecular weight. Their Tₘₐₓ were both in the range of 370˜376° C. (FIGS. 69˜72, corresponding to Embodiments 79, 82, 83, and 84, respectively). The T_{d,5%} of the sample prepared in Embodiment 79 using ^{t}Bu-P₄ as the catalyst and BnOH as the initiator was 20-30° C. lower than that of the sample prepared using only KOMe (Embodiments 82-84) as initiator. The difference may be caused by the significant difference in molecular weights of the polymers obtained from these two systems.

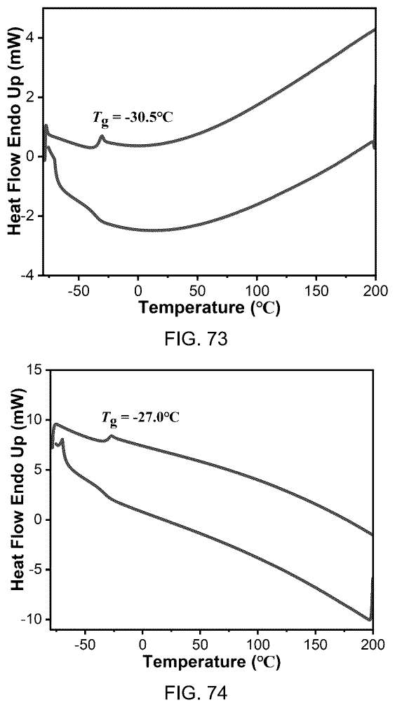

The DSC curves showed that the Tg of poly(δLH₂) prepared in Embodiments 79 and 84 were-30.5 and −27.0° C., respectively (FIGS. 73-74, corresponding to Embodiments 6 and 11, respectively), and the second heating and first cooling curves of the samples at a speed of 10° C./min only showed the glass transition temperatures (Tg) without the crystallization peaks as well as endothermic peaks, indicating that poly(δLH₂) materials were amorphous polymers.

### Application Example 1: Appearance and Light Transmission of Poly(δLH₂) Sample

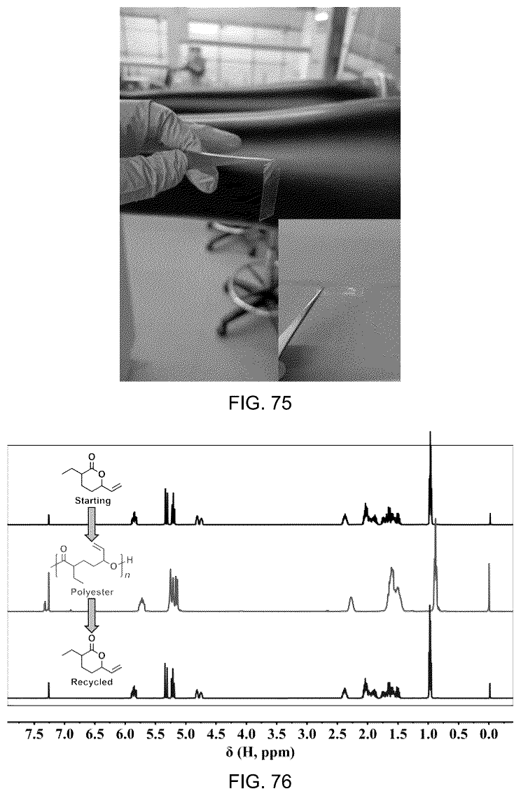

To verify the appearance and light transmittance of the poly(δLH₂) sample, the sample from Embodiment 85 in the present disclosure was subjected to hot pressing to obtain a strip. The strip was flexible, colorless transparent, and had a good ductility (FIG. 75).

### Application Example 2: Chemical Recycling

The chemical recyclability (Embodiment 91) of poly(δLH₂) sample was investigated in the present disclosure. The sample to be tested was poly(δLH₂) (Mₙ=11.7 kg mol⁻¹, f)=1.10) prepared in Embodiment 81. The polymer sample and 5 mol % of La[N(SiMe₃)₂]₃ serving as a catalyst were added to toluene, the initial theoretical concentration of the monomer was controlled to be [δLH₂]₀=0.18 M, and the system was then heated at 120° C. for 2 h. Later, the polymer sample was completely degraded to δLH₂ monomer.

### Embodiment 91: Chemical Recycling Experiment

In a glove box under nitrogen atmosphere, 200 mg of poly(δLH₂) sample (prepared from Embodiment 81) and 5 mol % La[N(SiMe₃)₂]₃ were added to a 50 mL Schlenk tube, and 7.2 mL of toluene was added for their dissolution. The Schlenk tube was then sealed with a rubber stopper, wrapped with a sealing film, taken out of the glove box, and placed in a pre-set 120° C. oil bath. After heating and stirring for 2 h, the solvent in the system was removed, and 10 mg of degraded product was subjected to ¹H NMR analysis for δLH₂ yield (see FIG. 76).

### Application Example 3: Post-Polymerization Modification

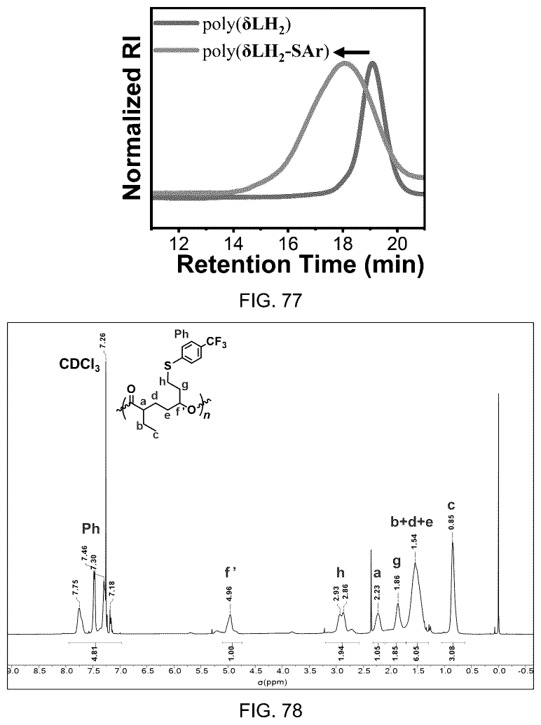

Due to the presence of a large number of olefin functional groups in the side chains of poly(δLH₂), the properties of the polymer can be controlled by post-polymerization modification. One effective method for post-polymerization modification is the photo-induced thiol-ene click chemistry reaction. The present disclosure involved preparing a solution of poly(δLH₂) sample, p-trifluoromethylbenzenethiol, and benzoin dimethyl ether, which was then coated to form a uniform film. The film was exposed to UV light at wavelengths of 254 nm and 365 nm for 12 hours (Embodiment 92). According to NMR analysis, it was found that about 87% of the olefin functional groups had reacted. And an obvious increase in molecular weight of the polymer sample was observed from the GPC curve (FIG. 77). In addition, the NMR hydrogen spectroscopy signals revealed a significant attenuation of the olefin signals in the poly(δLH₂-SAr) samples, along with the emergence of characteristic signals for the phenyl and thioether bond (FIG. 78).

### Embodiment 92: Photo-Initiated Thiol-Ene Click Chemistry Reaction

100 mg of poly(δLH₂) sample (prepared from Embodiment 81), 5.0 equivalent amounts of p-trifluoromethylbenzenethiol, and 10 mol % benzoin dimethyl ether were added to a 10 mL sample vial, and 0.5 mL of dichloromethane was added for their dissolution. After stirring for about 15 min, the solution was cast onto the top of a circular PTFE mold. Once the solvent evaporated, a film was formed, and then exposed to UV light at wavelengths of 254 nm and 365 nm overnight. After irradiation for a sufficiently long period of time, the film was dissolved in a small amount of dichloromethane, then reprecipitated several times with n-hexane and dried. The resulting product was poly(δLH₂-SAr) after post-polymerization modification.

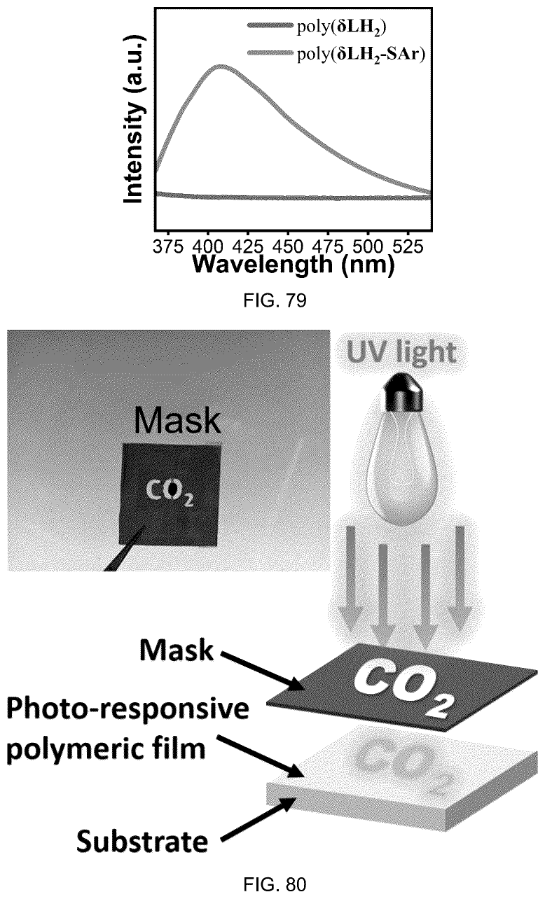

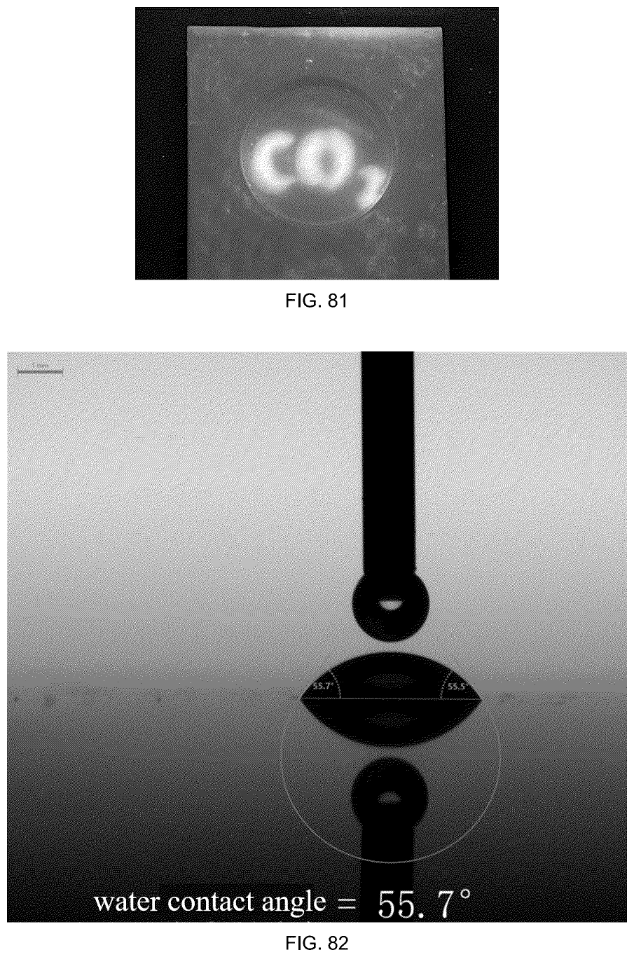

Interestingly, it was found that the film produced very distinct blue fluorescence under UV light at a wavelength of 365 nm during the photo-initiated thiol-olefin click chemistry reaction. Thus, the optical properties of the synthesized poly(δLH₂-SAr) were further characterized using photoluminescence spectra in the present disclosure. Under UV excitation at a wavelength of 360 nm, a photoluminescence peak was detected at about 410 nm (FIG. 79), in contrast, the poly(δLH₂) sample did not exhibit such luminescence. The patterning experiment shown in FIG. 80 demonstrated the ease of this optical modification. In the present disclosure, a mask with a hollowed-out “CO₂” pattern in the center was placed on top of the photoresponsive film (which contains poly(δLH₂), p-trifluoromethylbenzenethiol, and benzoin dimethyl ether) and subjected to UV light overnight. After irradiation, a “CO₂” fluorescent pattern appeared on the photoresponsive film, while the area covered by the mask was non-fluorescent (FIG. 81). This demonstrated that the fluorescence is attributable to the successful thiol-olefin click chemistry reaction.

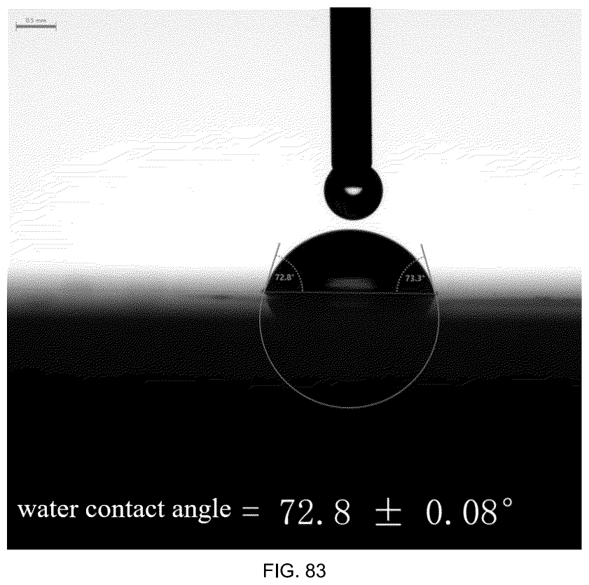

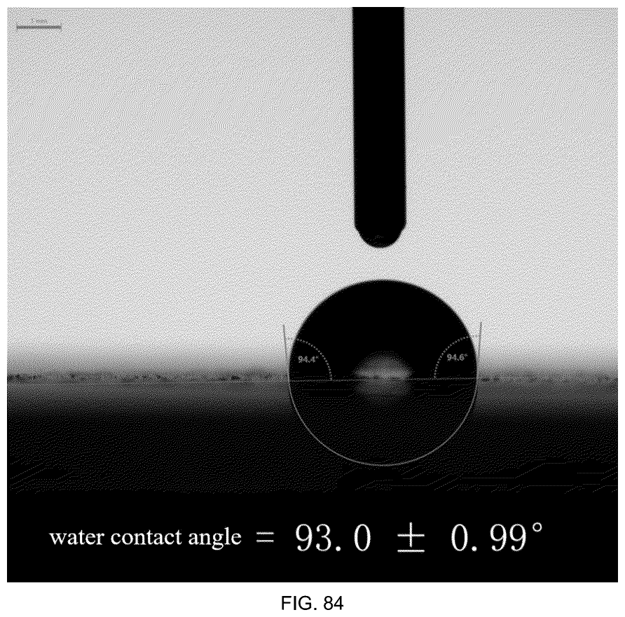

Likewise, the post-modification is effective in altering the hydrophilicity/hydrophobicity of the polymer. The contact angle of ultrapure water with the glass substrate was tested to be 55.7° (FIG. 82), whereas the contact angle of poly(δLH₂) with ultrapure water was 72.8±0.08° (FIG. 83). Additionally, the contact angle of poly(δLH₂-SAr) containing fluorine atoms with ultrapure water even reached 93.0±0.99° (FIG. 84). Perhaps it was the grafting of the trifluoromethyl group onto the polymer side chains that leads to the significantly more hydrophobic properties of the post-modified poly(δLH₂-SAr).

### Embodiment 93: Synthesis of polyurethane from HL monomers via a one-pot, two-step process

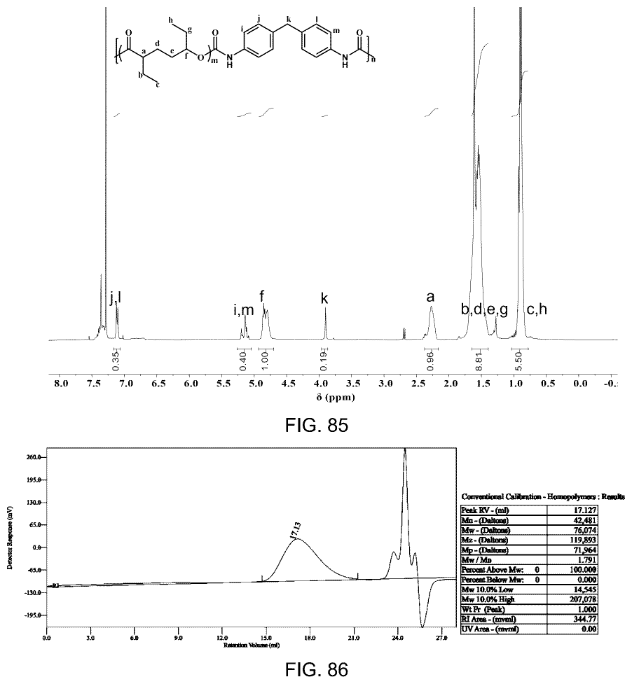

In a glove box under nitrogen atmosphere, 0.0063 mmol of ^{t}Bu-P₄ catalyst and 0.042 mmol of 1,4-BDM were added to a flame-dried 10 mL Schlenk tube, and then 0.02 mL of tetrahydrofuran was added for their dissolution. The Schlenk tube was then sealed with a rubber stopper, wrapped with a sealing film, taken out of the glove box, and placed in a pre-set −25° C. ice bath until the temperature stabilized. 0.63 mmol of HL was rapidly injected into the Schlenk tube using a syringe. After stirring for 12 h, 0.0063 mmol of diphenyl phosphate solution was added to quench the reaction. The system was transferred to 50° C. until the temperature stabilized, and then maintained for 5 min. 23.39 mg of 4,4′-methylene bis(phenyl isocyanate) was rapidly added to the system using a syringe, and then was quickly placed in the −25° C. ice bath for 24 h. After the reaction was completed, 5 mL of tetrahydrofuran was added for dilution. The mixture was added dropwise into a large amount of pre-cooled 0° C. methanol, and centrifuged at 10000 rpm for 5 min. The supernatant was discarded, and the precipitate was used for the subsequent centrifugation. The above centrifugation steps were repeated 3 times, and the precipitate collected in the last time was dried at 60° C. for 12 h under vacuum to obtain the polyurethane prepared from HL monomers. The NMR and GPC of polyurethane synthesized from HL monomers via one-pot two-step method were shown in FIG. 85 and FIG. 86.

In summary, the method for preparing CO₂-based polyester polymer of the present disclosure effectively utilizes carbon dioxide and mitigates the greenhouse effect by using cheap bulk materials as raw materials. The polyester polymer is chemically recyclable and has excellent physicochemical properties. The method of the present disclosure utilizes cheap and readily available carbon dioxide, 1,3-butadiene to synthesize a saturated lactone (HL) and a bisubstituted lactone (δLH₂), and then synthesizes a completely new CO₂-based recyclable polymer material by anionic ring-opening polymerization for the first time. The obtained polyHL material has better mechanical properties, is colorless and transparent, and can be used as a pressure-sensitive adhesive. The obtained poly(δLH₂) material also has good physical properties and chemically tunable properties, thus having the potential to be used in a variety of application scenarios. Both the polymers can be chemically recycled to obtain the corresponding monomers under certain conditions, establishing a closed-loop circular material economy. These methods not only avoid the damage of white pollution to the environment, but also provide a brand new solution for carbon dioxide reforming and reuse, and at the same time, meet the urgent needs of carbon neutral production of new polymers and circular material economy.

Therefore, the present disclosure effectively overcomes the shortcomings of the prior art and has high industrial utilization value.

The foregoing embodiments are merely illustrative of the principles and efficacies of the present disclosure, and are not intended to limit the present disclosure. Any person familiar with the art may modify or change the above embodiments without violating the spirit and scope of the present disclosure. Therefore, all equivalent modifications or changes made by persons having ordinary knowledge in the art without departing from the spirit and technical ideas disclosed in the present disclosure shall be covered by the claims of the present disclosure.

1. A polyester polymer compound, comprising repeating units shown as formula 4 below: 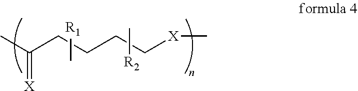
wherein R₁, R₂ are each independently selected from any one of hydrogen, halogen, alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; wherein R₁ and R₂ are the same or different and exist simultaneously;
wherein X is a heteroatom;
and wherein n is a positive integer no less than 1.

2. The polyester polymer compound of claim 1, wherein the alkyl, alkenyl, alkynyl, monocyclic aromatic group, polycyclic aromatic group, and polyheterocyclic aromatic group are mono-substituted or poly-substituted; and wherein substituent is independently selected from one or more of the following: hydrogen, heteroatom, amino, cyano, benzyl, alkyl carbonyl, alkenyl carbonyl, cycloalkyl carbonyl, phenyl carbonyl, benzyl carbonyl, alkoxycarbonyl, esteryl, sulfinyl, alkenyl, alkynyl, cycloalkyl, sulfonyl, hydroxyl, nitro, halogen, carboxyl, alkyl, alkoxyl, amine, cycloalkoxyl, cycloalkylamine group, sulfinamide, sulfonamide group, morpholinyl, and piperazinyl.

3. The polyester polymer compound of claim 1, wherein the polyester polymer compound is a carbon dioxide-based polyester poly(δLH₂), and the carbon dioxide-based polyester poly(δLH₂) comprises repeating units shown as formula 6 below: 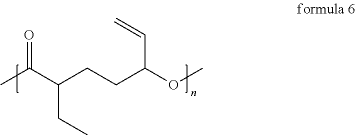
or wherein the polyester polymer compound is a carbon dioxide-based polyester poly(δHL), and the carbon dioxide-based polyester poly(δHL) comprises repeating units shown as formula 11 below: 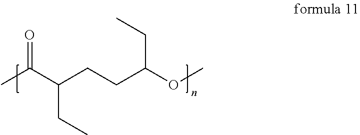
wherein n is a positive integer greater than or equal to 1.

4. The polyester polymer compound of claim 1, wherein the polyester polymer compound comprises a linear, cyclic, star-shaped topology.

5. The polyester polymer compound of claim 4, wherein the polyester polymer compound has a structure shown as formula 2 when it is linear: 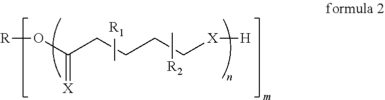
wherein R is selected from any one of alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol.

6. The polyester polymer compound of claim 5, wherein when the polyester polymer compound is a carbon dioxide-based polyester poly(δLH₂), the carbon dioxide-based polyester poly(δLH₂) has a structure shown as formula 7: 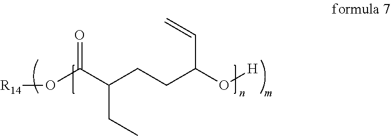
wherein R₁₄ is selected from C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
wherein n is a positive integer no less than 1,
and m is a positive integer no less than 1.

7. The polyester polymer compound of claim 4, wherein the polyester polymer compound has a structure shown as formula 3 when it is cyclic:

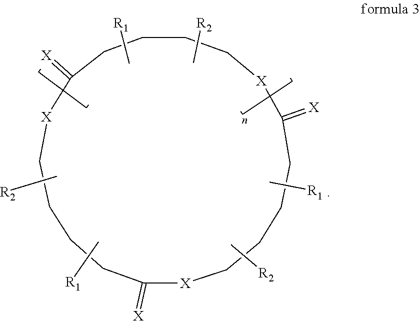

8. A use of the polyester polymer compound of claim 1 in the preparation of one or more of polyurethanes, polymer films, pressure sensitive adhesives, adhesive tapes, and thermoplastic elastomers.

9. (canceled)

10. A method for preparing a polyester polymer compound, comprising one or more of the follows:
A) when the polyester polymer compound is linear, the method comprises: obtaining the polyester polymer compound of formula 2 using heterocyclic lactones of formula 1 as raw materials under the catalysis of an organic base and the initiation of a reagent capable of providing an active proton R(OH)ₘ; wherein a corresponding reaction is shown as reaction formula I: 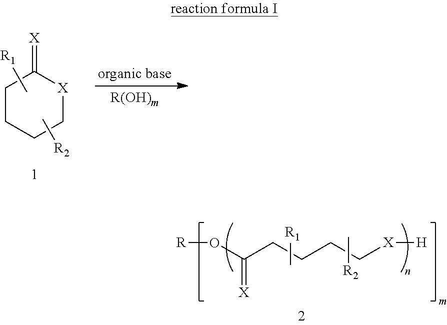
B) when the polyester polymer compound is cyclic, the method comprises: obtaining the polyester polymer compound of formula 3 using heterocyclic lactones of formula 1 as raw materials under the catalysis of an organic base; wherein a corresponding reaction is shown as reaction formula II: 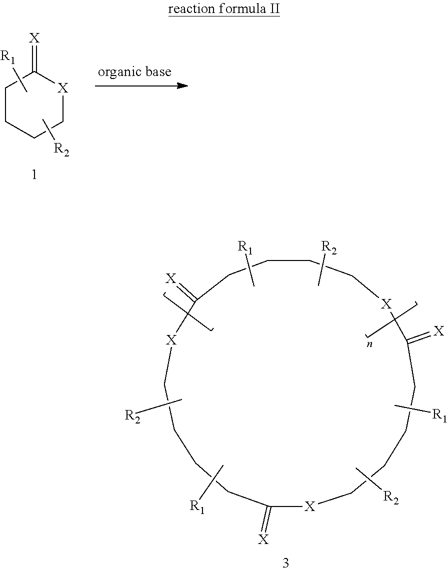
wherein in A) and/or B), R₁, R₂ are each independently selected from any one of hydrogen, halogen, alkyl, substituted alkyl (linear alkyl, branched alkyl, cycloalkyl), alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; wherein R¹ and R₂ are the same or different and exist simultaneously;
wherein X is a heteroatom;
wherein n is a positive integer no less than 1,
wherein the organic base comprises phosphazene, a compound containing a guanidine group, and a compound containing an amidino group;
wherein in B), R is selected from alkyl, substituted alkyl, alkenyl, substituted alkenyl, alkynyl, substituted alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
and wherein m is a positive integer no less than 1.

11. The method of claim 10, wherein in A), when the polyester polymer compound is a carbon dioxide-based polyester poly(δLH₂), the method comprises: obtaining the carbon dioxide-based polyester poly(δLH₂) of formula 7 using δLH₂ of formula 8 as raw materials under the catalysis of an organic base and the initiation of a reagent capable of providing an active proton R₁₄(OH)ₘ and/or (MO)ₘR₁₄; wherein a corresponding reaction is shown as reaction formula III: 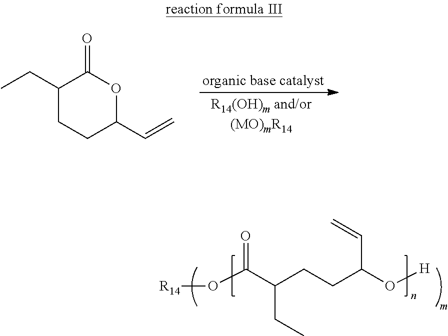
wherein in A), when the polyester polymer compound is a carbon dioxide-based polyester poly(δHL), the method comprises: obtaining the carbon dioxide-based polyester poly(δHL) of formula 12 using 3,6-diethyl-tetrahydro-2h-pyran-2-one as raw materials under the catalysis of an organic base and the initiation of a reagent capable of providing an active proton R₁₄(OH), and/or (MO)ₘR₁₄; wherein a corresponding reaction is shown as reaction formula III: 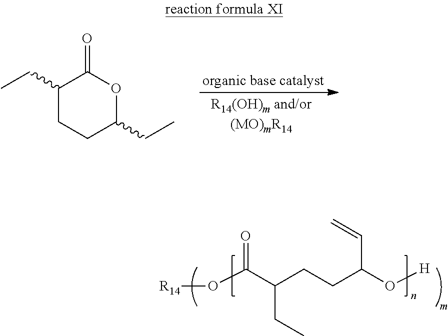
wherein R₁₄ is selected from C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
M is K, Na, Li, Rb or Cs;
wherein n is a positive integer no less than 1,
and m is a positive integer no less than 1;
and wherein the organic base is selected from one or more of phosphazene, a compound containing a guanidine group, and a compound containing an amidino group.

12. The method of claim 10, wherein the phosphazene is a strong Lewis base containing a structure of (R₂N)₂₋P═N; preferably, the phosphazene has a structure shown as formula 5: 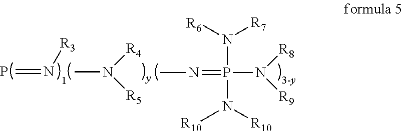
wherein in formula 5, R₃—R₁₀ each is independently selected from C1-C10 alkyl; and y is a positive integer no less than 1.

13. The method of claim 10, wherein the phosphazene is selected from ^{t}Bu-P₁, ^{t}Bu-P₂, and ^{t}Bu-P₄, their corresponding structures are shown below, respectively:

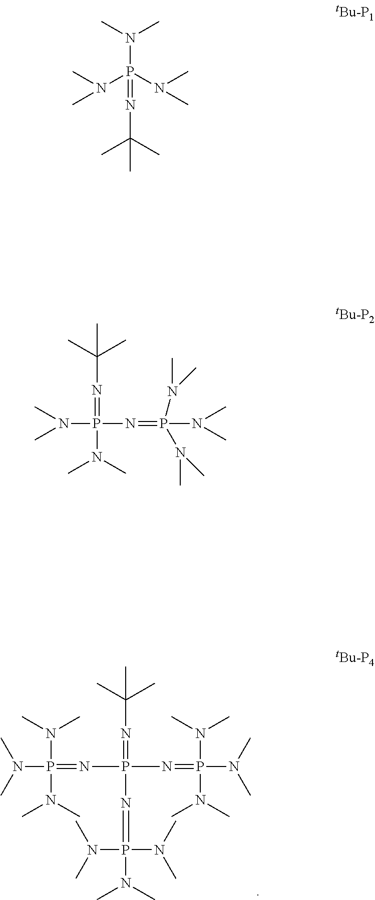

14. The method of claim 10, comprising any one or more of the following 1) to 11):
1) R₁ is an C1-C10 alkyl;
2) R₂ is an C1-C10 alkyl;
3) X is O, S, N or P;
4) in A), 1≤m≤10;
5) in A), a molar ratio of compound of formula 1, organic base, and initiator is (5-200):(0.01-5):1;
6) in B), a molar ratio of the compound of formula 1 and the organic base is (10-500):(0.01-5);
7) reaction is carried out under solvent-free conditions or under solvent conditions;
when the reaction is carried out under solvent conditions, solvent is selected from tetrahydrofuran (THF), benzene, toluene, xylene, dichlorobenzene, mesitylene, dichloromethane, chloroform, 1,2-dichloroethane, tetrahydropyrrole, tetrapyran, hexahydropyridine, ethyl acetate, ethyl ether, dimethyl ether, methyl ethyl ether, n-hexane, cyclohexane, cyclopentane, acetonitrile, dioxane, N,N-dimethylformamide, dimethyl sulphoxide;
8) in A), a reaction temperature ranges from −100 to 200° C.;
9) in A), a reaction time ranges from 10 s to 360 h;
10) in B), a reaction temperature ranges from −120 to 220° C.;
11) in B), a reaction time ranges from 5 s to 400 h.

15. The method of claim 10, comprising any one or more of the following i) to x):
i) R₁ is ethyl and R₂ is ethyl, i.e. HL of formula 1;
ii) the phosphazene is ^{t}Bu-P₄;
iii) in A), initiator is an alcohol; preferably, the initiator is an alkoxide; more preferably, the initiator is phenyl methanol (BnOH), 1,4-benzenedimethanol (1,4-BDM), 1,3-propylene glycol, 1,4-butanediol, potassium methoxide (KOMe), sodium methoxide (NaOMe), potassium tert-butoxide (KO^{t}Bu), sodium tert-butoxide (NaO^{t}Bu), or lithium tert-butoxide (LiO^{t}Bu);
iv) in A), a molar ratio of compound of formula 1, organic base, and initiator is (25-100):(0.1-1):1;
v) in B), a molar ratio of the compound of formula 1 and the organic base is (20-400):(0.1-4);
vi) a reaction solvent is THF;
vii) in A), a reaction temperature ranges from −80 to 200° C.;
viii) in A), a reaction time ranges from 1 min to 120 h;
xi) in B), a reaction temperature ranges from −80 to 160° C.;
xi) in B), a reaction time ranges from 5 min to 80 h.

16. The method of claim 11, comprising any one or more of the following a1)˜a2):
a1) the initiator is one or more of phenyl methanol (BnOH), potassium methoxide (KOMe), sodium methoxide (NaOMe), potassium tert-butoxide (KO^{t}Bu), sodium tert-butoxide (NaO^{t}Bu), and lithium tert-butoxide (LiO^{t}Bu);
a2) the molar ratio the compound of formula 1, the organic base, and the initiator is (5-200):(0-5):(0-1), and neither the amount of the organic base nor the amount of the initiator is 0; preferably, the molar ratio of the compound of formula 1, the organic base, and the initiator is (25-200):(0-1):(0-1); and neither the amount of the organic base nor the amount of the initiator is 0.

17. A polyester polymer compound, wherein the polyester polymer compound is prepared by the method of claim 10.

18. (canceled)

19. (canceled)

20. (canceled)

21. A method for catalyzing a polyester polymer compound of formula 2 and/or formula 3 to recycle heterocyclic monomers, comprising one or more of the follows:
a) catalyzing the polyester polymer compound of formula 2 using a catalytic method or a pyrolysis method; 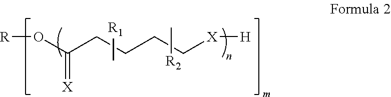
b) catalyzing the polyester polymer compound of formula 3 using an inorganic salt pyrolysis catalytic method or a La[N(SiMe₃)₂]₃ mild catalytic method; 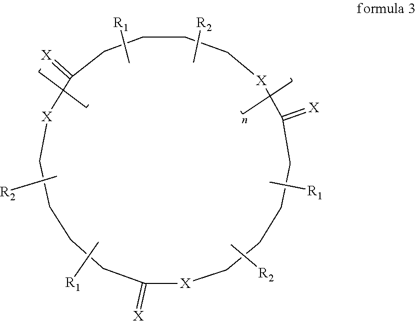
in a), the catalytic method comprises: degrading the polyester polymer compound of formula 2 by using an inorganic salt, a metal organic compound, or an organic compound catalyst, to obtain heterocyclic monomer of formula 1, or an oligomer or a derivative thereof;
the pyrolysis method comprises: heating and degrading the polyester polymer compound of formula 2 to obtain the heterocyclic monomer of formula 1, or an oligomer or a derivative thereof;
in b), the inorganic salt pyrolysis catalytic method comprises: catalyzing and degrading the polyester polymer compound of formula 3 by using an inorganic salt to obtain the heterocyclic monomer of formula 1, or an oligomer or a derivative thereof;
the La[N(SiMe₃)₂]₃ mild catalytic method comprises: catalyzing and degrading the polyester polymer compound of formula 3 by using La[N(SiMe₃)₂]₃ under mild conditions, to obtain the heterocyclic monomer of formula 1, or an oligomer or a derivative thereof.

22. The method of claim 21, wherein in a), when the polyester polymer compound is carbon dioxide-based polyester poly(δLH₂);
the catalytic method comprises: degrading the carbon dioxide-based polyester poly(δLH₂) of formula 8 by using an inorganic salt, a metal organic compound, or an organic compound catalyst, to obtain δLH₂ of formula 7, or an oligomer or a derivative thereof;
the pyrolysis method comprises: heating and degrading the carbon dioxide-based polyester poly(δLH₂) of formula 8 to obtain δLH₂ of formula 7, or an oligomer or a derivative thereof.

23. (canceled)

24. (canceled)

25. (canceled)

26. A disubstituted α,β-saturated six-membered cyclic lactone, having a structure of formula 10: 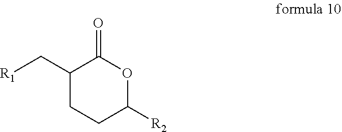
wherein R₁, R₂ are each independently selected from any one of hydrogen, halogen, C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, and substituted polyheterocyclic aromatic group, wherein the monocyclic aromatic group is selected from phenyl, aza-aromatic group, thio-aromatic group, and oxa-aromatic group, the polycyclic aromatic group and polyheterocyclic aromatic group refer to groups comprising two or more monocyclic aromatic groups; wherein R¹ and R₂ are the same or different.

27. The polyester polymer compound of claim 3, wherein when the polyester polymer compound is the carbon dioxide-based polyester poly(δHL), the carbon dioxide-based polyester poly(δHL) has a structure shown as formula 12: 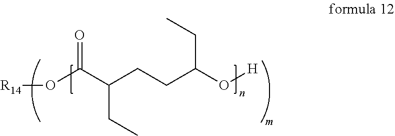
wherein R₁₄ is selected from C1-C20 alkyl, substituted C1-C20 alkyl, C2-C20 alkenyl, substituted C2-C20 alkenyl, C2-C20 alkynyl, substituted C2-C20 alkynyl, monocyclic aromatic group, substituted monocyclic aromatic group, polycyclic aromatic group, substituted polycyclic aromatic group, polyheterocyclic aromatic group, substituted polyheterocyclic aromatic group, and polymer groups with molecular weights no greater than 100,000 g/mol and containing repeating units of polyvinyl alcohol and/or polyethylene glycol;
wherein n is a positive integer no less than 1,
and m is a positive integer no less than 1.
